SEQUENCE LISTING (205 sequences)

SEQ ID NO: 1 - protein, 8 aa, artificial sequence
  CDR1 B1, 1E5, 1B11, C3CP, 1G12,G5CP, 1G9, 1H8, 1B6, 1F10, 1G1, G3CP
GANYGLAA

SEQ ID NO: 2 - protein, 8 aa, artificial sequence
  CDR1 1E2
GANYDLSA

SEQ ID NO: 3 - protein, 8 aa, artificial sequence
  CDR1 2G5, 2F4, G11CP, E6CP, A10CP
GANYGLSA

SEQ ID NO: 4 - protein, 8 aa, artificial sequence
  CDR1 B6CP, F2CP, D9CP
GANYDLAA

SEQ ID NO: 5 - protein, 8 aa, artificial sequence
  CDR1 G4′s
DANYGLAA

SEQ ID NO: 6 - protein, 10 aa, artificial sequence
  Consensus sequence HV2
SSNQERISIS

SEQ ID NO: 7 - protein, 10 aa, artificial sequence
  Consensus sequence HV2
SSNKERISIS

SEQ ID NO: 8 - protein, 5 aa, artificial sequence
  Consensus sequence HV4
NKRTM

SEQ ID NO: 9 - protein, 5 aa, artificial sequence
  Consensus sequence HV4
NKGTM

SEQ ID NO: 10 - protein, 14 aa, artificial sequence
  CDR3 B1, G11CP, D9CP
YPWGAGAPWLVQWY

SEQ ID NO: 11 - protein, 14 aa, artificial sequence
  CDR3 1E2
YPSGAGAPRPVQWY

SEQ ID NO: 12 - protein, 14 aa, artificial sequence
  CDR3 1E5
YPWGAGAPCLVQWY

SEQ ID NO: 13 - protein, 14 aa, artificial sequence
  CDR3 1B11
YPWGAGAPRLVQWY

SEQ ID NO: 14 - protein, 14 aa, artificial sequence
  CDR3 C3CP
YPWGAGAPRQVQWY

SEQ ID NO: 15 - protein, 14 aa, artificial sequence
  CDR3 2G5, 1G12
YPWGAGAPRSVQWY

SEQ ID NO: 16 - protein, 14 aa, artificial sequence
  CDR3 G5CP
YPWGAGAPSLVQWY

SEQ ID NO: 17 - protein, 14 aa, artificial sequence
  CDR3 2F4
YPWGAGAPSNVQWY

SEQ ID NO: 18 - protein, 14 aa, artificial sequence
  CDR3 1G9
YPWGAGAPSQVQWY

SEQ ID NO: 19 - protein, 14 aa, artificial sequence
  CDR3 1H8
YPWGAGAPSSVQWY

SEQ ID NO: 20 - protein, 14 aa, artificial sequence
  CDR3 G3CP/G3CPG4 lacking L to N mutation
YPWGAGAPYLVQWY

SEQ ID NO: 21 - protein, 14 aa, artificial sequence
  CDR3 1F10, E6CP, F2CP
YPWGAGAPWQVQWY

SEQ ID NO: 22 - protein, 14 aa, artificial sequence
  CDR3 B6CP, 1G1, A10CP
YPWGAGAPWSVQWY

SEQ ID NO: 23 - protein, 14 aa, artificial sequence
  CDR3 CDR3 G3CP and G3CPG4
YPWGAGAPYNVQWY

SEQ ID NO: 24 - protein, 14 aa, artificial sequence
  CDR3 G3CP/G3CPG4 lacking W to Y mutation i.e. 1B6
YPWGAGAPWNVQWY

SEQ ID NO: 25 - protein, 8 aa, artificial sequence
  CDR1 AE3 DeltaSer
GTRYGLYS

SEQ ID NO: 26 - protein, 9 aa, artificial sequence
  CDR1 AE3.S
GTRYGLYSS

SEQ ID NO: 27 - protein, 8 aa, artificial sequence
  CDR1 NAC6 DeltaSer
DTRYALYS

SEQ ID NO: 28 - protein, 9 aa, artificial sequence
  CDR1 NAC6.S
DTRYALYSS

SEQ ID NO: 29 - protein, 8 aa, artificial sequence
  CDR1 NAG8 DeltaSer
GTKYGLYA

SEQ ID NO: 30 - protein, 9 aa, artificial sequence
  CDR1 NAG8.S, AF7.S
GTKYGLYAS

SEQ ID NO: 31 - protein, 10 aa, artificial sequence
  HV2 AE3
SSDEERISIS

SEQ ID NO: 32 - protein, 10 aa, artificial sequence
  HV2 NAC6
STDEERISIG

SEQ ID NO: 33 - protein, 10 aa, artificial sequence
  HV2 NAG8
SPNKDRMIIG

SEQ ID NO: 34 - protein, 10 aa, artificial sequence
  HV2 AF7
STDKERIIIG

SEQ ID NO: 35 - protein, 5 aa, artificial sequence
  HV4 AE3
NKGTK

SEQ ID NO: 36 - protein, 5 aa, artificial sequence
  HV4 NAC6
NKGSK

SEQ ID NO: 37 - protein, 5 aa, artificial sequence
  HV4 NAG8
NNGTK

SEQ ID NO: 38 - protein, 5 aa, artificial sequence
  HV4 AF7
NNRSK

SEQ ID NO: 39 - protein, 12 aa, artificial sequence
  CDR3 PRA1 variants
REARHPWLRQWY

SEQ ID NO: 40 - protein, 25 aa, artificial sequence
  Consensus sequence FW1
ASVNQTPRTATKETGESLTINCVVT

SEQ ID NO: 41 - protein, 25 aa, artificial sequence
  Consensus sequence FW1
TRVDQSPSSLSASVGDRVTITCVLT

SEQ ID NO: 42 - protein, 25 aa, artificial sequence
  Consensus sequence FW1
ASVTQSPRSASKETGESLTITCRVT

SEQ ID NO: 43 - protein, 9 aa, artificial sequence
  Consensus sequnce FW2
TYWYRKNPG

SEQ ID NO: 44 - protein, 7 aa, artificial sequence
  Consensus sequence FW3a
GRYVESV

SEQ ID NO: 45 - protein, 7 aa, artificial sequence
  Consensus sequence FW3a
GRYSESV

SEQ ID NO: 46 - protein, 21 aa, artificial sequence
  Consensus sequence FW3b
SFTLTISSLQPEDSATYYCRA

SEQ ID NO: 47 - protein, 21 aa, artificial sequence
  Consensus sequence FW3b
SFSLRISSLTVEDSATYYCKA

SEQ ID NO: 48 - protein, 10 aa, artificial sequence
  Consensus sequence FW4
DGAGTVLTVN

SEQ ID NO: 49 - protein, 10 aa, artificial sequence
  Consensus sequence FW4
DGAGTKVEIK

SEQ ID NO: 50 - protein, 109 aa, artificial sequence
  G3CP
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVN

SEQ ID NO: 51 - protein, 109 aa, artificial sequence
  B1G4
TRVDQSPSSLSASVGDRVTITCVLTDANYGLAATYWYRKNPGSSNKERISISGRYSESVNKGTMSFTLTISSLQPEDSATYYCRAYPWGAGAPWLVQWYDGAGTKVEIK

SEQ ID NO: 52 - protein, 109 aa, artificial sequence
  1E2
ASVNQTPRTATKETGESLTINCVVTGANYDLSATYWYRKNPGSSNQERISISGRYVESVNKRTMSFSLRIKDLTVADSATYYCKAYPSGAGAPRPVQWYDGAGTVLTVN

SEQ ID NO: 53 - protein, 109 aa, artificial sequence
  1E5
ASVNQTPRTATKETGESLTINCVVTGANYGLAATYWYRKNPGSSNQERISISGRYVESVNKRTMSFSLRIKDLTVADSATYYCKAYPWGAGAPCLVQWYDGAGTVLTVN

SEQ ID NO: 54 - protein, 109 aa, artificial sequence
  1B11
ASVNQTPRTATKETGESLTINCVVTGANYGLAATYWYRKNPGSSNQERISISGRYVESVNKRTMSFSLRIKDLTVADSATYYCKAYPWGAGAPRLVQWYDGAGTVLTVN

SEQ ID NO: 55 - protein, 109 aa, artificial sequence
  C3CP
ASVNQTPRTATKETGESLTINCVVTGANYGLAATYWYRKNPGSSNQERISISGRYVESVNKRTMSFSLRIKDLTVADSATYYCKAYPWGAGAPRQVQWYDGAGTVLTVN

SEQ ID NO: 56 - protein, 109 aa, artificial sequence
  2G5
ASVNQTPRTATKETGESLTINCVVTGANYGLSATYWYRKNPGSSNQERISISGRYVESVNKRTMSFSLRIKDLTVADSATYYCKAYPWGAGAPRSVQWYDGAGTVLTVN

SEQ ID NO: 57 - protein, 109 aa, artificial sequence
  1G12
ASVNQTPRTATKETGESLTINCVVTGANYGLAATYWYRKNPGSSNQERISISGRYVESVNKRTMSFSLRIKDLTVADSATYYCKAYPWGAGAPRSVQWYDGAGTVLTVN

SEQ ID NO: 58 - protein, 109 aa, artificial sequence
  G5CP
ASVNQTPRTATKETGESLTINCVVTGANYGLAATYWYRKNPGSSNQERISISGRYVESVNKRTMSFSLRIKDLTVADSATYYCKAYPWGAGAPSLVQWYDGAGTVLTVN

SEQ ID NO: 59 - protein, 109 aa, artificial sequence
  2F4
ASVNQTPRTATKETGESLTINCVVTGANYGLSATYWYRKNPGSSNQERISISGRYVESVNKRTMSFSLRIKDLTVADSATYYCKAYPWGAGAPSNVQWYDGAGTVLTVN

SEQ ID NO: 60 - protein, 109 aa, artificial sequence
  1G9
ASVNQTPRTATKETGESLTINCVVTGANYGLAATYWYRKNPGSSNQERISISGRYVESVNKRTMSFSLRIKDLTVADSATYYCKAYPWGAGAPSQVQWYDGAGTVLTVN

SEQ ID NO: 61 - protein, 109 aa, artificial sequence
  1H8
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVN

SEQ ID NO: 62 - protein, 109 aa, artificial sequence
  G11CP
ASVNQTPRTATKETGESLTINCVVTGANYGLSATYWYRKNPGSSNQERISISGRYVESVNKRTMSFSLRIKDLTVADSATYYCKAYPWGAGAPWLVQWYDGAGTVLTVN

SEQ ID NO: 63 - protein, 109 aa, artificial sequence
  D9CP
ASVNQTPRTATKETGESLTINCVVTGANYDLAATYWYRKNPGSSNQERISISGRYVESVNKRTMSFSLRIKDLTVADSATYYCKAYPWGAGAPWLVQWYDGAGTVLTVN

SEQ ID NO: 64 - protein, 109 aa, artificial sequence
  1B6
ASVNQTPRTATKETGESLTINCVVTGANYGLAATYWYRKNPGSSNQERISISGRYVESVNKRTMSFSLRIKDLTVADSATYYCKAYPWGAGAPWNVQWYDGAGTVLTVN

SEQ ID NO: 65 - protein, 109 aa, artificial sequence
  1F10
ASVNQTPRTATKETGESLTINCVVTGANYGLAATYWYRKNPGSSNQERISISGRYVESVNKRTMSFSLRIKDLTVADSATYYCKAYPWGAGAPWQVQWYDGAGTVLTVN

SEQ ID NO: 66 - protein, 109 aa, artificial sequence
  E6CP
ASVNQTPRTATKETGESLTINCVVTGANYGLSATYWYRKNPGSSNQERISISGRYVESVNKRTMSFSLRIKDLTVADSATYYCKAYPWGAGAPWQVQWYDGAGTVLTVN

SEQ ID NO: 67 - protein, 109 aa, artificial sequence
  F2CP
ASVNQTPRTATKETGESLTINCVVTGANYDLAATYWYRKNPGSSNQERISISGRYVESVNKRTMSFSLRIKDLTVADSATYYCKAYPWGAGAPWQVQWYDGAGTVLTVN

SEQ ID NO: 68 - protein, 109 aa, artificial sequence
  B6CP
ASVNQTPRTATKETGESLTINCVVTGANYDLAATYWYRKNPGSSNQERISISGRYVESVNKRTMSFSLRIKDLTVADSATYYCKAYPWGAGAPWSVQWYDGAGTVLTVN

SEQ ID NO: 69 - protein, 109 aa, artificial sequence
  1G1
ASVNQTPRTATKETGESLTINCVVTGANYGLAATYWYRKNPGSSNQERISISGRYVESVNKRTMSFSLRIKDLTVADSATYYCKAYPWGAGAPWSVQWYDGAGTVLTVN

SEQ ID NO: 70 - protein, 109 aa, artificial sequence
  A10CP
ASVNQTPRTATKETGESLTINCVVTGANYGLSATYWYRKNPGSSNQERISISGRYVESVNKRTMSFSLRIKDLTVADSATYYCKAYPWGAGAPWSVQWYDGAGTVLTVN

SEQ ID NO: 71 - protein, 109 aa, artificial sequence
  G3CP G4
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIK

SEQ ID NO: 72 - protein, 109 aa, artificial sequence
  G3CP V15
ASVTQSPRSASKETGESLTITCRVTGANYGLAATYWYRKNPGSSNQERISISGRYSESVNKRTMSFSLRISSLTVEDSATYYCKAYPWGAGAPYNVQWYDGQGTKLEVK

SEQ ID NO: 73 - protein, 109 aa, artificial sequence
  1H8 G4
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIK

SEQ ID NO: 74 - protein, 109 aa, artificial sequence
  1H8 V15
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVK

SEQ ID NO: 75 - protein, 109 aa, artificial sequence
  C3CP G4
TRVDQSPSSLSASVGDRVTITCVLTDANYGLAATYWYRKNPGSSNKERISISGRYSESVNKGTMSFTLTISSLQPEDSATYYCRAYPWGAGAPRQVQWYDGAGTKVEIK

SEQ ID NO: 76 - protein, 109 aa, artificial sequence
  C3CP V15
ASVTQSPRSASKETGESLTITCRVTGANYGLAATYWYRKNPGSSNQERISISGRYSESVNKRTMSFSLRISSLTVEDSATYYCKAYPWGAGAPRQVQWYDGQGTKLEVK

SEQ ID NO: 77 - protein, 107 aa, artificial sequence
  P3A1 G1 AE3
TRVDQSPSSLSASVGDRVTITCVLTGTRYGLYSTYWYRKNPGSSDEERISISGRYSESVNKGTKSFTLTISSLQPEDSATYYCRAREARHPWLRQWYDGAGTKVEIK

SEQ ID NO: 78 - protein, 108 aa, artificial sequence
  P3A1 G1 AE3.S
TRVDQSPSSLSASVGDRVTITCVLTGTRYGLYSSTYWYRKNPGSSDEERISISGRYSESVNKGTKSFTLTISSLQPEDSATYYCRAREARHPWLRQWYDGAGTKVEIK

SEQ ID NO: 79 - protein, 107 aa, artificial sequence
  P3A1 G1 NAC6
TRVDQSPSSLSASVGDRVTITCVLTDTRYALYSTYWYRKNPGSTDEERISIGGRYSESVNKGSKSFTLTISSLQPEDSATYYCRAREARHPWLRQWYDGAGTKVEIK

SEQ ID NO: 80 - protein, 108 aa, artificial sequence
  P3A1 G1 NAC6.S
TRVDQSPSSLSASVGDRVTITCVLTDTRYALYSSTYWYRKNPGSTDEERISIGGRYSESVNKGSKSFTLTISSLQPEDSATYYCRAREARHPWLRQWYDGAGTKVEIK

SEQ ID NO: 81 - protein, 107 aa, artificial sequence
  P3A1 G1 NAG8
TRVDQSPSSLSASVGDRVTITCVLTGTKYGLYATYWYRKNPGSPNKDRMIIGGRYSESVNNGTKSFTLTISSLQPEDSATYYCRAREARHPWLRQWYDGAGTKVEIK

SEQ ID NO: 82 - protein, 108 aa, artificial sequence
  P3A1 G1 NAG8.S
TRVDQSPSSLSASVGDRVTITCVLTGTKYGLYASTYWYRKNPGSPNKDRMIIGGRYSESVNNGTKSFTLTISSLQPEDSATYYCRAREARHPWLRQWYDGAGTKVEIK

SEQ ID NO: 83 - protein, 108 aa, artificial sequence
  P3A1 G1 AF7.S
TRVDQSPSSLSASVGDRVTITCVLTGTKYGLYASTYWYRKNPGSTDKERIIIGGRYSESVNNRSKSFTLTISSLQPEDSATYYCRAREARHPWLRQWYDGAGTKVEIK

SEQ ID NO: 84 - protein, 21 aa, artificial sequence
  Consensus sequence FW3b
SFSLRIKDLTVADSATYYCKA

SEQ ID NO: 85 - protein, 10 aa, artificial sequence
  Consensus sequence FW4
DGQGTKLEVK

SEQ ID NO: 86 - protein, 15 aa, artificial sequence
  Linker [G4S]3
GGGGSGGGGSGGGGS

SEQ ID NO: 87 - protein, 25 aa, artificial sequence
  Linker [G4S]5
GGGGSGGGGSGGGGSGGGGSGGGGS

SEQ ID NO: 88 - protein, 7 aa, artificial sequence
  Linker
PGVQPSP

SEQ ID NO: 89 - protein, 12 aa, artificial sequence
  Linker
PGVQPSPGGGGS

SEQ ID NO: 90 - protein, 12 aa, artificial sequence
  Linker
PGVQPAPGGGGS

SEQ ID NO: 91 - protein, 15 aa, artificial sequence
  Linear peptide sequence
YMESLHMQGEIENQI

SEQ ID NO: 92 - protein, 20 aa, artificial sequence
  Linear peptide sequence
CQPWNSQYPHTHTFTALRFP

SEQ ID NO: 93 - protein, 23 aa, artificial sequence
  Linear peptide sequence
RSTIYGSRLRIRNLDTTDTGYFQ

SEQ ID NO: 94 - protein, 36 aa, artificial sequence
  Linear peptide sequence
QCVATNGKEVVSSTGVLFVKFGPPPTASPGYSDEYE

SEQ ID NO: 95 - protein, 103 aa, artificial sequence
  BA11
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIK

SEQ ID NO: 96 - protein, 13 aa, artificial sequence
  Linker Wobbe-G5S
PGVQPSPGGGGGS

SEQ ID NO: 97 - protein, 25 aa, artificial sequence
  Cys containing C-terminal tag
QACKAHHHHHHGAEFEQKLISEEDL

SEQ ID NO: 98 - protein, 25 aa, artificial sequence
  Consensus sequence C-terminal tag
QASGAHHHHHHGAEFEQKLISEEDL

SEQ ID NO: 99 - protein, 25 aa, artificial sequence
  Consensus sequence C-terminal tag
QACGAHHHHHHGAEFEQKLISEEDL

SEQ ID NO: 100 - protein, 23 aa, artificial sequence
  Consensus sequence C-terminal tag
AAAHHHHHHGAEFEQKLISEEDL

SEQ ID NO: 101 - protein, 23 aa, artificial sequence
  Consensus sequence C-terminal tag
ACAHHHHHHGAEFEQKLISEEDL

SEQ ID NO: 102 - protein, 11 aa, artificial sequence
  Consensus sequence C-terminal tag
QASGAHHHHHH

SEQ ID NO: 103 - protein, 11 aa, artificial sequence
  Consensus sequence C-terminal tag
QACGAHHHHHH

SEQ ID NO: 104 - protein, 11 aa, artificial sequence
  Consensus sequence C-terminal tag
QACKAHHHHHH

SEQ ID NO: 105 - protein, 9 aa, artificial sequence
  Consensus sequence C-terminal tag
AAAHHHHHH

SEQ ID NO: 106 - protein, 9 aa, artificial sequence
  Consensus sequence C-terminal tag
ACAHHHHHH

SEQ ID NO: 107 - protein, 5 aa, artificial sequence
  Consensus sequence C-terminal tag
QASGA

SEQ ID NO: 108 - protein, 5 aa, artificial sequence
  Consensus sequence C-terminal tag
QACGA

SEQ ID NO: 109 - protein, 5 aa, artificial sequence
  Consensus sequence C-terminal tag
QACKA

SEQ ID NO: 110 - protein, 3 aa, artificial sequence
  Consensus sequence C-terminal tag
ACA

SEQ ID NO: 111 - protein, 5 aa, artificial sequence
  Consensus sequence C-terminal tag
SAPSA

SEQ ID NO: 112 - protein, 10 aa, artificial sequence
  C-terminal tag with c-Myc sequence
EQKLISEEDL

SEQ ID NO: 113 - protein, 109 aa, artificial sequence
  B1
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVN

SEQ ID NO: 114 - protein, 107 aa, artificial sequence
  P3A1 G1
TRVDQSPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIK

SEQ ID NO: 115 - protein, 109 aa, artificial sequence
  B1V15
ASVTQSPRSASKETGESLTITCRVTGANYGLAATYWYRKNPGSSNQERISISGRYSESVNKRTMSFSLRISSLTVEDSATYYCKAYPWGAGAPWLVQWYDGQGTKLEVK

SEQ ID NO: 116 - protein, 35 aa, artificial sequence
  Linker [G4S]7
GGGGSGGGGSGGGGSGGGGSGGGGSGGGGSGGGGS

SEQ ID NO: 117 - protein, 368 aa, artificial sequence
  B1-G4-[WGM]-CD3
TRVDQSPSSLSASVGDRVTITCVLTDANYGLAATYWYRKNPGSSNKERISISGRYSESVNKGTMSFTLTISSLQPEDSATYYCRAYPWGAGAPWLVQWYDGAGTKVEIKPGVQPAPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 118 - protein, 368 aa, artificial sequence
  G3CP-[WGM]-CD3
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNPGVQPAPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 119 - protein, 368 aa, artificial sequence
  G3CPG4-[WGM]-CD3
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKPGVQPAPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 120 - protein, 366 aa, artificial sequence
  P3A1G1AE3-[WGM]-CD3
TRVDQSPSSLSASVGDRVTITCVLTGTRYGLYSTYWYRKNPGSSDEERISISGRYSESVNKGTKSFTLTISSLQPEDSATYYCRAREARHPWLRQWYDGAGTKVEIKPGVQPAPGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 121 - protein, 476 aa, artificial sequence
  B1G4-[WGM]-BA11-[G4S]-CD3
TRVDQSPSSLSASVGDRVTITCVLTDANYGLAATYWYRKNPGSSNKERISISGRYSESVNKGTMSFTLTISSLQPEDSATYYCRAYPWGAGAPWLVQWYDGAGTKVEIKPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 122 - protein, 476 aa, artificial sequence
  G3CP [WGM] BA11-[G4S]-CD3
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 123 - protein, 476 aa, artificial sequence
  G3CPG4 [WGM] BA11-[G4S]-CD3
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 124 - protein, 474 aa, artificial sequence
  P3A1G1AE3 [WGM] BA11-[G4S]-CD3
TRVDQSPSSLSASVGDRVTITCVLTGTRYGLYSTYWYRKNPGSSDEERISISGRYSESVNKGTKSFTLTISSLQPEDSATYYCRAREARHPWLRQWYDGAGTKVEIKPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 125 - protein, 480 aa, artificial sequence
  P3A1 [WGM] G3CP-[G4S]-CD3
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 126 - protein, 480 aa, artificial sequence
  P3A1 [WGM] G3CPG4-[G4S]-CD3
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSTRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 127 - protein, 480 aa, artificial sequence
  P3A1G1AE3 [WGM] G3CP-[G4S]-CD3
TRVDQSPSSLSASVGDRVTITCVLTDTRYALYSTYWYRKNPGSTDEERISIGGRYSESVNKGSKSFTLTISSLQPEDSATYYCRAREARHPWLRQWYDGAGTKVEIKPGVQPAPGGGGSASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 128 - protein, 480 aa, artificial sequence
  P3A1G1AE3 [WGM] G3CPG4-[G4S]-CD3
TRVDQSPSSLSASVGDRVTITCVLTDTRYALYSTYWYRKNPGSTDEERISIGGRYSESVNKGSKSFTLTISSLQPEDSATYYCRAREARHPWLRQWYDGAGTKVEIKPGVQPAPGGGGSTRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 129 - protein, 483 aa, artificial sequence
  P3A1G1AE3 [WGM] D3-[G4S]-CD3
TRVDQSPSSLSASVGDRVTITCVLTDTRYALYSTYWYRKNPGSTDEERISIGGRYSESVNKGSKSFTLTISSLQPEDSATYYCRAREARHPWLRQWYDGAGTKVEIKPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 130 - protein, 243 aa, artificial sequence
  UCL OKT3 sequence (WO2019008379)
QVQLVQSGAEVKKPGSSVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTMVTVSSVEGGSGGSGGSGGSGGVDDIQMTQSPSSLSASVGDRVTITCSASSSVSYMNWYQQKPGKAPKRLIYDTSKLASGVPSRFSGSGSGTEFTLTISSLQPEDFATYYCQQWSSNPFTFGQGTKVEIK

SEQ ID NO: 131 - protein, 243 aa, artificial sequence
  Harpoon ID20 (WO2016187594)
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 132 - protein, 107 aa, artificial sequence
  DPK9
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIK

SEQ ID NO: 133 - DNA, 20 bp, artificial sequence
  Primer 280
ctaccgtggcccaggcggcc

SEQ ID NO: 134 - DNA, 24 bp, artificial sequence
  Primer 287
ggtgatggtgggcccctgaggcct

SEQ ID NO: 135 - protein, 132 aa, artificial sequence
  P3A1 G1
TRVDQTPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIKQASGAHHHHHHGAEFEQKLISEEDL

SEQ ID NO: 136 - protein, 133 aa, artificial sequence
  NAG8.S
TRVDQSPSSLSASVGDRVTITCVLTGTKYGLYASTYWYRKNPGSPNKDRMIIGGRYSESVNNGTKSFTLTISSLQPEDSATYYCRAREARHPWLRQWYDGAGTKVEIKQASGAHHHHHHGAEFEQKLISEEDL

SEQ ID NO: 137 - protein, 133 aa, artificial sequence
  AC5.S
TRVDQSPSSLSASVGDRVTITCVLTDTSYGLYSSTYWYRKNPGSSNKERMSISGRYSESVNKGSKSFTLTISSLQPEDSATYYCRAREARHPWLRQWYDGAGTKVEIKQASGAHHHHHHGAEFEQKLISEEDL

SEQ ID NO: 138 - protein, 133 aa, artificial sequence
  AG5.S
TRVDQSPSSLSASVGDRVTITCVLTDTNYALYSSTYWYRKNPGSTNKESMSIGGRYSESVNKRSKSFTLTISSLQPEDSATYYCRAREARHPWLRQWYDGAGTKVEIKQASGAHHHHHHGAEFEQKLISEEDL

SEQ ID NO: 139 - protein, 133 aa, artificial sequence
  AF1.S
TRVDQSPSSLSASVGDRVTITCVLTDAKYGLYSSTYWYRKNPGSPDKERIINGGRYSESVNNRTKSFTLTISSLQPEDSATYYCRAREARHPWLRQWYDGAGTKVEIKQASGAHHHHHHGAEFEQKLISEEDL

SEQ ID NO: 140 - protein, 133 aa, artificial sequence
  AB11.S
TRVDQSPSSLSASVGDRVTITCVLTDARYGLFASTYWYRKNPGSPDKDSISIGGRYSESVNKRSKSFTLTISSLQPEDSATYYCRAREARHPWLRQWYDGAGTKVEIKQASGAHHHHHHGAEFEQKLISEEDL

SEQ ID NO: 141 - protein, 133 aa, artificial sequence
  NAE1.S
TRVDQSPSSLSASVGDRVTITCVLTGTKYGLFSSTYWYRKNPGSSDKERISIGGRYSESVNKGPKSFTLTISSLQPEDSATYYCRAREARHPWLRQWYDGAGTKVEIKQASGAHHHHHHGAEFEQKLISEEDL

SEQ ID NO: 142 - protein, 133 aa, artificial sequence
  AA6.S
TRVDQSPSSLSASVGDRVTITCVLTGTRYGLYASTYWYRKNPGSPDEESMINGGRYSESVNKRTKSFTLTISSLQPEDSATYYCRAREARHPWLRQWYDGAGTKVEIKQASGAHHHHHHGAEFEQKLISEEDL

SEQ ID NO: 143 - protein, 133 aa, artificial sequence
  AB7.S
TRVDQSPSSLSASVGDRVTITCVLTDTKYALFSSTYWYRKNPGSSNEERISIGGRYSESVNNRTKSFTLTISSLQPEDSATYYCRAREARHPWLRQWYDGAGTKVEIKQASGAHHHHHHGAEFEQKLISEEDL

SEQ ID NO: 144 - protein, 133 aa, artificial sequence
  AF7.S
TRVDQSPSSLSASVGDRVTITCVLTGTKYGLYASTYWYRKNPGSTDKERIIIGGRYSESVNNRSKSFTLTISSLQPEDSATYYCRAREARHPWLRQWYDGAGTKVEIKQASGAHHHHHHGAEFEQKLISEEDL

SEQ ID NO: 145 - protein, 232 aa, artificial sequence
  Human IgG1 Fc (hFc)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 356 aa, artificial sequence
  G3CP hFc(S239C+Y407T)
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 147 - protein, 356 aa, artificial sequence
  G3CPG4 hFc(S239C+Y407T)
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 148 - protein, 354 aa, artificial sequence
  P3A1 hFc(S239C+T366Y)
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 241 aa, artificial sequence
  VH-[G4S]3-VL
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 150 - protein, 245 aa, artificial sequence
  VL-[G4S]3-VH
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 151 - protein, 380 aa, artificial sequence
  Human ROR1-His (CHO)
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEHHHHHH

SEQ ID NO: 152 - protein, 108 aa, artificial sequence
  Human ROR1 (Ig domain)
SELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKAAAHHHHH

SEQ ID NO: 153 - protein, 380 aa, artificial sequence
  Mouse ROR1-His (CHO)
QETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEHHHHHH

SEQ ID NO: 154 - protein, 616 aa, artificial sequence
  Human ROR1-Fc (CHO)
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 611 aa, artificial sequence
  Human ROR2-Fc (CHO)
EVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 616 aa, artificial sequence
  Mouse ROR1-Fc (CHO)
QETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 616 aa, artificial sequence
  Rat ROR1-Fc (CHO)
QETELSVSAELVPTSSWNTSSEIDKDSYLTLDEPMNNITTSLGQTAELHCKVSGNPPPNIRWFKNDAPVVQEPRRISFRATNYGSRLRIRNLDTTDTGYFQCVATSGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCHTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 248 aa, artificial sequence
  Figure 7
GGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 159 - protein, 247 aa, artificial sequence
  N-terminal VNAR hFc (linker-hFc sequence)
GGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 247 aa, artificial sequence
  Cysteine engineered variants for bioconjugation
GGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 247 aa, artificial sequence
  Cysteine engineered variants for bioconjugation
GGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 162 - protein, 252 aa, artificial sequence
  C-terminal hFc VNAR (hFc-linker sequence)
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGS

SEQ ID NO: 163 - protein, 12 aa, artificial sequence
  Intein fusions His tag
QACKAHHHHHHG

SEQ ID NO: 164 - protein, 26 aa, artificial sequence
  Intein fusions HisMyc tag
QACKAHHHHHHGAEFEQKLISEEDLG

SEQ ID NO: 165 - protein, 356 aa, artificial sequence
  G3CP hFc(Y407T)
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 166 - protein, 356 aa, artificial sequence
  G3CPG4 hFc(Y407T)
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 167 - protein, 354 aa, artificial sequence
  P3A1 hFc(T366Y)
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 133 aa, artificial sequence
  NAC1.S
TRVDQSPSSLSASVGDRVTITCVLTDTKYGLYSSTYWYRKNPGSTDEERIIIGGRYSESVNKGSKSFTLTISSLQPEDSATYYCRAREARHPWLRQWYDGAGTKVEIKQASGAHHHHHHGAEFEQKLISEEDL

SEQ ID NO: 169 - protein, 133 aa, artificial sequence
  NAC6.S
TRVDQSPSSLSASVGDRVTITCVLTDTRYALYSSTYWYRKNPGSTDEERISIGGRYSESVNKGSKSFTLTISSLQPEDSATYYCRAREARHPWLRQWYDGAGTKVEIKQASGAHHHHHHGAEFEQKLISEEDL

SEQ ID NO: 170 - protein, 133 aa, artificial sequence
  NAE9.S
TRVDQSPSSLSASVGDRVTITCVLTGTGYGLYSSTYWYRKNPGSTDEERMIISGRYSESVNKRTKSFTLTISSLQPEDSATYYCRAREARHPWLRQWYDGAGTKVEIKQASGAHHHHHHGAEFEQKLISEEDL

SEQ ID NO: 171 - protein, 133 aa, artificial sequence
  NAF3.S
TRVDQSPSSLSASVGDRVTITCVLTDTKYGLYASTYWYRKNPGSPDEERIIISGRYSESVNKGSKSFTLTISSLQPEDSATYYCRAREARHPWLRQWYDGAGTKVEIKQASGAHHHHHHGAEFEQKLISEEDL

SEQ ID NO: 172 - protein, 133 aa, artificial sequence
  NAF5.S
TRVDQSPSSLSASVGDRVTITCVLTDTKYALFSSTYWYRKNPGSTNEDRISIGGRYSESVNKGTKSFTLTISSLQPEDSATYYCRAREARHPWLRQWYDGAGTKVEIKQASGAHHHHHHGAEFEQKLISEEDL

SEQ ID NO: 173 - protein, 133 aa, artificial sequence
  AA5.S
TRVDQSPSSLSASVGDRVTITCVLTGTGYGLYSSTYWYRKNPGSSDEERIIIGGRYSESVNKGSKSFTLTISSLQPEDSATYYCRAREARHPWLRQWYDGAGTKVEIKQASGAHHHHHHGAEFEQKLISEEDL

SEQ ID NO: 174 - protein, 133 aa, artificial sequence
  AD10.S
TRVDQSPSSLSASVGDRVTITCVLTGTKYGLYSSTYWYRKNPGSSDKESIIIGGRYSESVNNGTKSFTLTISSLQPEDSATYYCRAREARHPWLRQWYDGAGTKVEIKQASGAHHHHHHGAEFEQKLISEEDL

SEQ ID NO: 175 - protein, 133 aa, artificial sequence
  AE3.S
TRVDQSPSSLSASVGDRVTITCVLTGTRYGLYSSTYWYRKNPGSSDEERISISGRYSESVNKGTKSFTLTISSLQPEDSATYYCRAREARHPWLRQWYDGAGTKVEIKQASGAHHHHHHGAEFEQKLISEEDL

SEQ ID NO: 176 - protein, 133 aa, artificial sequence
  AF4.S
TRVDQSPSSLSASVGDRVTITCVLTDTRYALYASTYWYRKNPGSTDEDRIIIGGRYSESVNKGSKSFTLTISSLQPEDSATYYCRAREARHPWLRQWYDGAGTKVEIKQASGAHHHHHHGAEFEQKLISEEDL

SEQ ID NO: 177 - protein, 13 aa, artificial sequence
  WobbeCys-G4S linker
PGVQPCPGGGGGS

SEQ ID NO: 178 - protein, 356 aa, artificial sequence
  G3CP-hFc(S239C)
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 356 aa, artificial sequence
  G3CPG4-hFc(S239C)
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 356 aa, artificial sequence
  1H8-hFc (S239C)
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 356 aa, artificial sequence
  1H8 G4-hFc (S239C)
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 356 aa, artificial sequence
  1H8 V15-hFc (S239C)
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 356 aa, artificial sequence
  1H8-hFc
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 356 aa, artificial sequence
  1H8 G4-hFc
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 356 aa, artificial sequence
  1H8 V15-hFc
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 356 aa, artificial sequence
  G3CP-hFc
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 356 aa, artificial sequence
  G3CPG4-hFc
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 356 aa, artificial sequence
  G3CP hFc(T366Y)
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 355 aa, artificial sequence
  G3CPG4 hFc (T366Y)
RVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 354 aa, artificial sequence
  P3A1 hFc (Y407T)
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 191 - protein, 356 aa, artificial sequence
  G3CP hFc(S239C+T366Y)
ASVNQTPRTATKETGESLTINCVVTGANYGLAATYWYRKNPGSSNQERISISGRYVESVNKRTMSFSLRIKDLTVADSATYYCKAYPWGAGAPYNVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 356 aa, artificial sequence
  G3CPG4 hFc (S239C+T366Y)
TRVDQSPSSLSASVGDRVTITCVLTDANYGLAATYWYRKNPGSSNKERISISGRYSESVNKGTMSFTLTISSLQPEDSATYYCRAYPWGAGAPYNVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 354 aa, artificial sequence
  P3A1 hFc (S239C+Y407T)
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 194 - protein, 356 aa, artificial sequence
  1H8 hFc (S239C+Y407T)
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 195 - protein, 356 aa, artificial sequence
  1H8 G4 hFc (S239C+Y407T)
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 196 - protein, 356 aa, artificial sequence
  1H8 v15 hFc (S239C+Y407T)
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 197 - protein, 356 aa, artificial sequence
  1H8 hFc (S239C+T366Y)
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 356 aa, artificial sequence
  1H8 G4 hFc (S239C+T366Y)
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 356 aa, artificial sequence
  1H8 v15 hFc (S239C+T366Y)
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 356 aa, artificial sequence
  1H8 hFc (Y407T)
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 201 - protein, 356 aa, artificial sequence
  1H8 G4 hFc (Y407T)
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 202 - protein, 356 aa, artificial sequence
  1H8 v15 hFc (Y407T)
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 203 - protein, 356 aa, artificial sequence
  1H8 hFc (T366Y)
ASVNQTPRTATKETGESLTINCVVTGANYGLAATYWYRKNPGSSNQERISISGRYVESVNKRTMSFSLRIKDLTVADSATYYCKAYPWGAGAPSSVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 356 aa, Artificial sequence
  1H8 G4 hFc (T366Y)
TRVDQSPSSLSASVGDRVTITCVLTDANYGLAATYWYRKNPGSSNKERISISGRYSESVNKGTMSFTLTISSLQPEDSATYYCRAYPWGAGAPSSVQWYDGAGTKVEIKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 356 aa, Artificial sequence
  1H8 v15 hFc (T366Y)
ASVTQSPRSASKETGESLTITCRVTGANYGLAATYWYRKNPGSSNQERISISGRYSESVNKRTMSFSLRISSLTVEDSATYYCKAYPWGAGAPSSVQWYDGQGTKLEVKGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0269051 A1
(43) Pub. Date: Aug. 28, 2025

(54) ROR1-SPECIFIC VARIANT ANTIGEN BINDING MOLECULES

(71) Applicant: ALMAC DISCOVERY LIMITED, Craigavon (GB)

(72) Inventors: Paul Richard TRUMPER, Craigavon (GB); Jennifer THOM, Craigavon (GB); Andrei KAMENSKI, Craigavon (GB); Graham John COTTON, Craigavon (GB); Caroline Jane BARELLE, Aberdeen (GB); Marina KOVALEVA, Aberdeen (GB); Andrew Justin Radcliffe PORTER, Aberdeen (GB)

(21) Appl. No.: 18/257,436

(22) PCT Filed: Dec. 17, 2021
(86) PCT No.: PCT/EP2021/086667

§ 371 Date: Jun. 14, 2023

(30) Foreign Application Priority Data
Dec. 18, 2020 (GB) 2020154.7

(51) Int. Cl. A61K 47/68; A61K 35/17; A61K 40/11; A61K 40/31; A61P 35/00; C07K 16/28

(52) U.S. Cl. CPC A61K 47/6809; A61K 35/17; A61K 40/11; A61K 40/31; A61K 47/6849; A61K 47/6889; A61P 35/00; C07K 16/2803; A61K 2239/13; A61K 2239/21; A61K 2239/22; C07K 2317/24; C07K 2317/33; C07K 2317/524; C07K 2317/526; C07K 2317/565; C07K 2317/567; C07K 2317/622; C07K 2317/73; C07K 2317/92

(57) ABSTRACT

The present invention relates to receptor tyrosine kinase-like orphan receptor 1 (ROR1) specific variant antigen binding molecules and associated fusion proteins, chimeric antigen receptors, nucleic acid sequences, vectors, host cells, pharmaceutical compositions, medical uses and conjugates, and methods of preparing and using the same.

### REFERENCE TO SEQUENCE LISTING SUBMITTED ELECTRONICALLY

The instant application contains a Sequence Listing which has been submitted electronically in text format and is hereby incorporated by reference in its entirety. Said text copy, created on Feb. 16, 2024, is named 18257436_SL.txt and is 291,785 bytes in size.

### FIELD OF INVENTION

The present invention relates to receptor tyrosine kinase-like orphan receptor 1 (ROR1) specific antigen binding molecules and associated fusion proteins and conjugates. In a further aspect, the present invention relates to conjugated immunoglobulin-like shark variable novel antigen receptors (VNARs).

### BACKGROUND

Receptor tyrosine kinase-like orphan receptor 1 (ROR1) is a 937 amino acid glycosylated type I single pass transmembrane protein. The extracellular region consists of three distinct domains composing an N-terminal immunoglobulin domain (Ig), followed by a cysteine rich fizzled domain (fz) which in turn is linked to the membrane proximal kringle domain (kr). The intracellular region of the protein contains a pseudo kinase domain followed by two Ser/Thr rich domains which are interspersed by a proline-rich region, and this same overall domain architecture is conserved in the closely related family member ROR2, with which it shares high sequence identity.

ROR1 is expressed during embryonic development, where it is prominently expressed in neural crest cells and in the necrotic and interdigital zones in the later stages of development. However, its expression is quickly silenced after birth, and is largely absent in normal adult tissue. ROR1 expression has been observed at both the mRNA and protein level across a broad range of solid tumours and haematological malignancies including lung, endometrial, pancreatic, ovarian, colon, head and neck and prostate cancers, melanoma and renal cell carcinoma, breast cancer and chronic lymphocytic leukemia (CLL) and acute lymphoblastic leukemia (AML). Additionally, increased ROR1 expression is reported to correlate with poor clinical outcomes for a number of cancer indications including breast cancer, ovarian cancer, colorectal cancer, lung adenocarcinoma and CLL.

Consistent with ROR1's expression pattern and the link to poor clinical prognosis, a functional role for ROR1 in tumorigenesis and disease progression has been demonstrated for a number of different cancer indications. ROR1 promotes epithelial-mesenchymal transition and metastasis in models of breast cancer and spheroid formation and tumour engraftment in models of ovarian cancer. ROR1 is a transcript target of the NKX2-1/TTF-1 lineage survival factor oncogene in lung adenocarcinoma, where it sustains EGFR signalling and represses pro-apoptotic signalling. ROR1 has also been shown to act as a scaffold to sustain caveolae structures and by-pass signalling mechanism that confer resistance to EGFR tyrosine kinase inhibitors. Signalling through an ROR1-HER3 complex modulates the Hippo-YAP pathway and promotes breast cancer bone metastasis and the protein can promote Met-driven tumorigenesis. ROR1 expression is associated with chemotherapy resistance in breast cancer through activation of Hippo-YAP/TAZ and BM11 pathways. Whilst in CLL, ROR1 has been reported to heterooligomerise with ROR2 in response to Wnt5a to transduce signalling and enhance proliferation and migration.

Given the functional role of ROR1 in cancer pathology and the general lack of expression on normal adult tissue, this oncofetal protein is an attractive target for cancer therapy. Antibodies to ROR1 have been described in the literature WO2021097313 (4A5 kipps), WO2014031174 (UC961), WO2016187220 (Five Prime) WO2010124188 (2A2), WO2012075158 (R¹¹, R¹²), WO2011054007 (Oxford Bio), WO2011079902 (Bioinvent) WO2017127664, WO2017127664 (NBE Therapeutics, SCRIPPS), WO2016094847 (Emergent), WO2017127499), and a humanised murine anti-ROR1 antibody, UC961, has entered clinical trials for relapsed or refractory chronic lymphocytic leukemia.

Chimeric antigen receptor T-cells targeting ROR1 have also been reported (Hudecek M et al, Clin. Cancer Res., 2013, 19, 3153-64) and preclinical primate studies with UC961 and with CAR-T cells targeting ROR1 showed no overt toxicity, which is consistent with the general lack of expression of the protein on adult tissue (Choi M et al, Clinical Lymphoma, myeloma & leukemia, 2015, S167; Berger C et al, Cancer Immunol. Res., 2015, 3, 206).

Single domain binding molecules can be derived from an array of proteins from distinct species. The immunoglobulin isotope novel antigen receptor (IgNAR) is a homodimeric heavy-chain complex originally found in the serum of the nurse shark (Ginglymostoma cirratum) and other sharks and ray species. IgNARs do not contain light chains and are distinct from the typical immunoglobulin structure. Each molecule consists of a single-variable domain (VNAR) and five constant domains (CNAR). The nomenclature in the literature refers to IgNARs as immunoglobulin isotope novel antigen receptors or immunoglobulin isotope new antigen receptors and the terms are synonymous.

There are three main defined types of shark IgNAR known as I, II and III (Kovalena et al, Exp Opin Biol Ther 2014 14(10) 1527-1539). These have been categorized based on the position of non-canonical cysteine residues which are under strong selective pressure and are therefore rarely replaced.

All three types have the classical immunoglobulin canonical cysteines at positions 35 and 107 that stabilize the standard immunoglobulin fold, together with an invariant tryptophan at position 36. There is no defined CDR2 as such, but regions of sequence variation that compare more closely to TCR HV2 and HV4 have been defined in framework 2 and 3 respectively. Type I has germline encoded cysteine residues in framework 2 and framework 4 and an even number of additional cysteines within CDR3. Crystal structure studies of a Type I IgNAR isolated against and in complex with lysozyme enabled the contribution of these cysteine residues to be determined. Both the framework 2 and 4 cysteines form disulphide bridges with those in CDR3 forming a tightly packed structure within which the CDR3 loop is held tightly down towards the HV2 region. To date Type I IgNARs have only been identified in nurse sharks—all other elasmobranchs, including members of the same order have only Type II or variations of this type.

Type II IgNAR are defined as having a cysteine residue in CDR1 and CDR3 which form intra-molecular disulphide bonds that hold these two regions in close proximity, resulting in a protruding CDR3 that is conducive to binding pockets or grooves. Type I sequences typically have longer CDR3s than type II with an average of 21 and 15 residues respectively. This is believed to be due to a strong selective pressure for two or more cysteine residues in Type I CDR3 to associate with their framework 2 and 4 counterparts. Studies into the accumulation of somatic mutations show that there are a greater number of mutations in CDR1 of type II than type I, whereas HV2 regions of Type I show greater sequence variation than Type II. This evidence correlates well with the determined positioning of these regions within the antigen binding sites. A third IgNAR type known as Type III has been identified in neonates. This member of the IgNAR family lacks diversity within CDR3 due to the germline fusion of the D1 and D2 regions (which form CDR3) with the V-gene. Almost all known clones have a CDR3 length of 15 residues with little or no sequence diversity.

Another structural type of VNAR, termed type (IIb or IV), has only two canonical cysteine residues (in framework 1 and framework 3b regions). So far, this type has been found primarily in dogfish sharks and was also isolated from semisynthetic V-NAR libraries derived from wobbegong sharks.

ROR1-specific antigen binding molecules, including VNARs, are described in WO 2019/122447, hereby incorporated by reference in its entirety. Amongst others, WO 2019/122447 describes the sequences of B1 and P3A1 G1 identified below.

B1
(SEQ ID NO: 113)
ASVNQTPRTATKETGESLTINCVVTGANYGLAATYWYRKNPGSSNQERIS
ISGRYVESVNKRTMSFSLRIKDLTVADSATYYCKAYPWGAGAPWLVQWYD
GAGTVLTVN
P3A1 G1
(SEQ ID NO: 114)
TRVDQSPSSLSASVGDRVTITCVLTDTSYGLYSTYWYRKNPGSSNKEQIS
ISGRYSESVNKGTKSFTLTISSLQPEDSATYYCRAREARHPWLRQWYDGA
GTKVEIK

Conjugates of ROR1-specific antigen binding molecules, including VNARs, are described in PCT/EP2020/067210 filed on 19 Jun. 2020, hereby incorporated by reference in its entirety. PCT/EP2020/067210 describes anthracycline (PNU) derivatives suitable for use in drug conjugates. Specifically, derivatives of PNU159682 are provided, which lack the C14 carbon and attached hydroxyl functionality, and in which an ethylenediamino (EDA) group forms part of a linker region between the C13 carbonyl of PNU159682 and a maleimide group. Alternatively, the same molecules may be described with EDA-PNU as the “warhead” such that the EDA group is not considered part of the linker region. Where the linker comprises val-cit-PAB the maleimide group may be replaced with any reactive group suitable for a conjugation reaction. Such payloads are able to react with a free thiol group on another molecule. Where the free thiol is on a protein a protein-drug conjugate (PDC) may be formed.

The anthracycline derivative PNU-159682 has been described as a metabolite of nemorubicin (Quintieri et al. (2005) Clin. Cancer Res. 11, 1608-1617) and has been reported to exhibit extremely high potency for in vitro cell killing in the pico- to femtomolar range with one ovarian (A2780) and one breast cancer (MCF7) cell line (WO2012/073217 A1). Derivatives of PNU-159682 have also been described in WO2016/102679.

Conjugation of PNU-159682 derivatives to antibodies is described in WO2009/099741, WO2016/127081 and WO2016/102679, Yu et al, Clin. Cancer Res 2015, 21, 3298 and Stefan et al, Mol. Cancer. Ther., 2017, 16, 879.

Described herein are ROR1-specific variant antigen binding molecules having improved properties and conjugates thereof to derivatives of PNU-159682.

### SUMMARY OF INVENTION

The present invention generally relates to specific antigen binding molecules.

According to a first aspect, the invention provides a receptor tyrosine kinase-like orphan receptor 1 (ROR1) specific antigen binding molecule comprising an amino acid sequence represented by the formula (I):

FW1-CDR1-FW2-HV2-FW3a-HV4-FW3b-CDR3-FW4 (I)
wherein
   CDR3 is a CDR sequence having an amino acid sequence selected from the group consisting of YPWGAGAPYNVQWY (SEQ ID NO: 23), YPWGAGAPYLVQWY (SEQ ID NO: 20), YPWGAGAPWNVQWY (SEQ ID NO: 24), YPSGAGAPRPVQWY (SEQ ID NO: 11), YPWGAGAPCLVQWY (SEQ ID NO: 12), YPWGAGAPRLVQWY (SEQ ID NO: 13), YPWGAGAPRQVQWY (SEQ ID NO: 14), YPWGAGAPRSVQWY (SEQ ID NO: 15), YPWGAGAPSLVQWY (SEQ ID NO: 16), YPWGAGAPSNVQWY (SEQ ID NO: 17), YPWGAGAPSQVQWY (SEQ ID NO: 18), YPWGAGAPSSVQWY (SEQ ID NO: 19), YPWGAGAPWQVQWY (SEQ ID NO: 21), YPWGAGAPWSVQWY (SEQ ID NO: 22), and YPWGAGAPWLVQWY (SEQ ID NO: 10);
   CDR1 is a CDR sequence having an amino acid sequence selected from the group consisting of GANYGLAA (SEQ ID NO: 1), DANYGLAA (SEQ ID NO: 5), GANYDLSA (SEQ ID NO: 2), GANYGLSA (SEQ ID NO: 3), and GANYDLAA (SEQ ID NO: 4)
   FW1 is a framework region;
   FW2 is a framework region;
   HV2 is a hypervariable sequence having an amino acid sequence selected from the group consisting of SSNQERISIS (SEQ ID NO: 6), and SSNKERISIS (SEQ ID NO: 7);
   FW3a is a framework region;
   HV4 is a hypervariable sequence having an amino acid sequence selected from the group consisting of NKRTM (SEQ ID NO: 8) and NKGTM (SEQ ID NO: 9);
   FW3b is a framework region;
   FW4 is a framework region;
wherein if CDR3 is YPWGAGAPWLVQWY (SEQ ID NO: 10) then CDR1 is selected from the group consisting of DANYGLAA (SEQ ID NO: 5), GANYGLSA (SEQ ID NO: 3) and GANYDLAA (SEQ ID NO: 4).

According to a second aspect, the invention provides a receptor tyrosine kinase-like orphan receptor 1 (ROR1) specific antigen binding molecule comprising an amino acid sequence represented by the formula (I):

FW1-CDR1-FW2-HV2-FW3a-HV4-FW3b-CDR3-FW4 (I)
wherein
   CDR1 is a CDR sequence having an amino acid sequence selected from the group consisting of GTRYGLYS (SEQ ID NO: 25), GTRYGLYSS (SEQ ID NO: 26), DTRYALYS (SEQ ID NO: 27), DTRYALYSS (SEQ ID NO: 28), GTKYGLYA (SEQ ID NO: 29) and GTKYGLYAS (SEQ ID NO: 30);
   FW1 is a framework region;
   FW2 is a framework region;
   HV2 is a hypervariable sequence having an amino acid sequence selected from the group consisting of SSDEERISIS (SEQ ID NO: 31), STDEERISIG (SEQ ID NO: 32), SPNKDRMIIG (SEQ ID NO: 33), and STDKERIIIG (SEQ ID NO: 34);
   FW3a is a framework region;
   HV4 is a hypervariable sequence having an amino acid sequence selected from the group consisting of NKGTK (SEQ ID NO: 35), NKGSK (SEQ ID NO: 36), NNGTK (SEQ ID NO: 37), and NNRSK (SEQ ID NO: 38);
   FW3b is a framework region;
   CDR3 is a CDR sequence having an amino acid sequence according to REARHPWLRQWY (SEQ ID NO: 39);
   FW4 is a framework region.

According to a third aspect, the invention provides a recombinant fusion protein comprising a specific antigen binding molecule according to the first or the second aspects of the invention.

According to a fourth aspect, the invention provides a recombinant fusion protein comprising an antigen binding molecule comprising an amino acid sequence represented by the formula (I):

FW1-CDR1-FW2-HV2-FW3a-HV4-FW3b-CDR3-FW4 (I)
wherein
   FW1 is a framework region
   CDR1 is a CDR sequence
   FW2 is a framework region
   HV2 is a hypervariable sequence
   FW3a is a framework region
   HV4 is a hypervariable sequence
   FW3b is a framework region
   CDR3 is a CDR sequence
   FW4 is a framework region
or a functional variant thereof,
wherein the antigen binding molecule is fused to a fragment of an immunoglobulin Fc region wherein the fragment of an immunoglobulin Fc region is engineered to dimerize with a second fragment of an immunoglobulin Fc region.

According to a fifth aspect, the invention provides a recombinant fusion protein dimer comprising
(a) a first recombinant fusion protein, wherein the first recombinant fusion protein is a recombinant fusion protein according to the third or fourth aspects, and
(b) a second recombinant fusion protein, wherein the second recombinant fusion protein comprises a second antigen binding molecule fused to a second fragment of an immunoglobulin Fc region engineered to dimerize with the first fragment of an immunoglobulin Fc region.

According to a sixth aspect, the invention provides a ROR1-specific chimeric antigen receptor (CAR), comprising at least one ROR1-specific antigen binding molecule as defined by the first or second aspects of the invention, fused or conjugated to at least one transmembrane region and at least one intracellular domain.

The present invention also provides a cell comprising a chimeric antigen receptor according to the sixth aspect, which cell is preferably an engineered T-cell.

In a seventh aspect of the invention, there is provided a nucleic acid sequence comprising a polynucleotide sequence that encodes a specific antigen binding molecule, recombinant fusion protein, recombinant fusion protein dimer or chimeric antigen receptor according to the first, second, third, fourth, fifth or sixth aspects of the invention.

There is also provided a vector comprising a nucleic acid sequence in accordance with the seventh aspect and a host cell comprising such a nucleic acid.

A method for preparing a specific antigen binding molecule, recombinant fusion protein, recombinant fusion protein dimer or chimeric antigen receptor, of the first, second, third, fourth, fifth or sixth aspect is provided, the method comprising cultivating or maintaining a host cell comprising the polynucleotide or vector described above under conditions such that said host cell produces the specific antigen binding molecule, recombinant fusion protein or chimeric antigen receptor, optionally further comprising isolating the specific antigen binding molecule, recombinant fusion protein, recombinant fusion protein dimer or chimeric antigen receptor.

In an eighth aspect of the invention, there is provided a pharmaceutical composition comprising the specific antigen binding molecule, fusion protein, recombinant fusion protein dimer or chimeric antigen receptor of the first, second, third, fourth, fifth or sixth aspects. The pharmaceutical composition may contain a variety of pharmaceutically acceptable carriers. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The specific antigen binding molecule, recombinant fusion protein, recombinant fusion protein dimer or chimeric antigen receptor of the first, second, third, fourth, fifth or sixth aspects may be for use in therapy. More specifically, the specific antigen binding molecule, recombinant fusion protein, recombinant fusion protein dimer or chimeric antigen receptor of the first, second, third, fourth, fifth or sixth aspects may be for use in the treatment of cancer. Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is the use of a specific antigen binding molecule, recombinant fusion protein, recombinant fusion protein dimer or chimeric antigen receptor of the first, second, third, fourth, fifth or sixth aspects in the manufacture of a medicament for the treatment of a disease in a patient in need thereof.

The specific antigen binding molecule, recombinant fusion protein, recombinant fusion protein dimer or chimeric antigen receptor of the first, second, third, fourth, fifth, sixth aspects or pharmaceutical composition of the eighth aspect may be administered in a single dose. As used herein “single dose” refers to a dosage regiment consisting of one dose. Alternatively, a multi-dose regiment may be used. Without being bound by theory, the advantages of the specific binding molecule, recombinant fusion protein, recombinant fusion protein dimer or chimeric antigen receptor of the first, second, third, fourth, fifth, sixth aspects or pharmaceutical composition of the eighth aspect may be particularly apparent when administered in a single dose.

Furthermore, in accordance with the present invention there is provided a method of treatment of a disease in a patient in need of treatment comprising administration to said patient of a therapeutically effective dosage of a specific antigen binding molecule, recombinant fusion protein, recombinant fusion protein dimer or chimeric antigen receptor of the first, second, third, fourth, fifth or sixth aspects or a pharmaceutical composition of the eighth aspect.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is a method of assaying for the presence of a target analyte in a sample, comprising the addition of a detectably labelled specific antigen binding molecule of the first aspect or second aspect, or a recombinant fusion protein of the third or fourth aspect, or a recombinant fusion protein dimer of the fifth aspect, to the sample and detecting the binding of the molecule to the target analyte.

In addition, there is provided herein a method of imaging a site of disease in a subject, comprising administration of a detectably labelled specific antigen binding molecule of the first aspect or second aspect, or a detectably labelled recombinant fusion protein of the third or fourth aspect, or a recombinant fusion protein dimer of the fifth aspect to a subject.

There is also provided herein a method of diagnosis of a disease or medical condition in a subject comprising administration of a specific antigen binding molecule of the first aspect or second aspect, or a recombinant fusion protein of the third or fourth aspect, or a recombinant fusion protein dimer of the fifth aspect.

Also contemplated herein is an antibody, antibody fragment or antigen-binding molecule that competes for binding to ROR1 with the ROR1-specific antigen binding molecule of the first or second aspect. The term “compete” when used in the context of antigen binding proteins (e.g., neutralizing antigen binding proteins or neutralizing antibodies) means competition between antigen binding proteins as determined by an assay in which the antigen binding protein (e.g., antibody or functional fragment thereof) under test prevents or inhibits specific binding of a the antigen binding molecule defined herein (e.g., specific antigen binding molecule of the first aspect) to a common antigen (e.g., ROR1 in the case of the specific antigen binding molecule of the first or second aspect).

Also described herein is a kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises a ROR1-specific antigen binding molecule of the first or second aspect, a recombinant fusion protein of the third or fourth aspect, or a recombinant fusion protein dimer of the fifth aspect, a chimeric antigen receptor of the sixth aspect or a nucleic acid sequence of the seventh aspect, each being optionally derivatized, wherein presence of antigen in the sample suggests that the subject suffers from cancer. Preferably the antigen comprises ROR1 protein, more preferably an extracellular domain thereof. More preferably, the kit is used to identify the presence or absence of ROR1-positive cells in the sample, or determine the concentration thereof in the sample. The kit may also comprise a positive control and/or a negative control against which the assay is compared and/or a label which may be detected.

The present invention also provides a method for diagnosing a subject suffering from cancer, or a predisposition thereto, or for providing a prognosis of the subject's condition, the method comprising detecting the concentration of antigen present in a sample obtained from a subject, wherein the detection is achieved using a ROR1-specific antigen binding molecule of the first or second aspect, a recombinant fusion protein of the third or fourth aspect, or a recombinant fusion protein dimer of the fifth aspect, a chimeric antigen receptor of the sixth aspect or a nucleic acid sequence of the seventh aspect, each being optionally derivatized, and wherein presence of antigen in the sample suggests that the subject suffers from cancer.

Also contemplated herein is a method of killing or inhibiting the growth of a cell expressing ROR1 in vitro or in a patient, which method comprises administering to the cell a pharmaceutically effective amount or dose of (i) ROR1-specific antigen binding molecule of the first or second aspect, a recombinant fusion protein of the third or fourth aspect, or a recombinant fusion protein dimer of the fifth aspect, a nucleic acid sequence of the seventh aspect, or the CAR or cell according to the sixth aspect, or (ii) of a pharmaceutical composition of the eighth aspect. Preferably, the cell expressing ROR1 is a cancer cell. More preferably, the ROR1 is human ROR1.

According to a ninth aspect, the invention provides a specific antigen binding molecule comprising an amino acid sequence represented by the formula (II):

X-FW1-CDR1-FW2-HV2-FW3a-HV4-FW3b-CDR3-FW4-Y (II)
wherein
   FW1-CDR1-FW2-HV2-FW3a-HV4-FW3b-CDR3-FW4 is a ROR1-specific antigen binding molecule according to the first or second aspect
   X and Y are optional amino acid sequences
wherein the specific antigen binding molecule is conjugated to a second moiety.

According to a tenth aspect, the invention provides a target-binding molecule-drug conjugate, comprising
(a) a ROR1 specific antigen binding molecule according to the first, second or ninth aspect, or a recombinant fusion protein of the third, or fourth aspect or a recombinant fusion protein dimer of the fifth aspect, and
(b) an anthracycline (PNU) derivative, wherein the target-binding molecule-drug conjugate has the structure of formula (III):

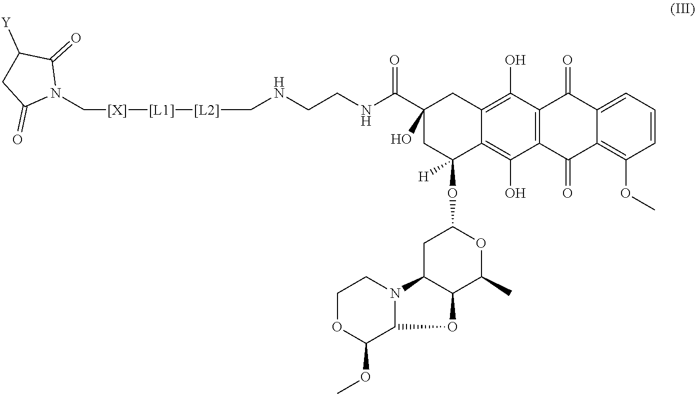

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting ofvaline (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises a ROR1 specific antigen binding molecule according to the first, second or ninth aspect, or a recombinant fusion protein of the third or fourth aspect, or a recombinant fusion protein dimer of the fifth aspect.

According to an eleventh aspect, the invention provides a target-binding molecule-drug conjugate, comprising
(a) a ROR1 specific antigen binding molecule according to the first, second or ninth aspect, or a recombinant fusion protein according to the third or fourth aspect, or a recombinant fusion protein dimer of the fifth aspect, and
(b) an anthracycline (PNU) derivative,
wherein the target-binding molecule-drug conjugate has the structure of formula (IV):

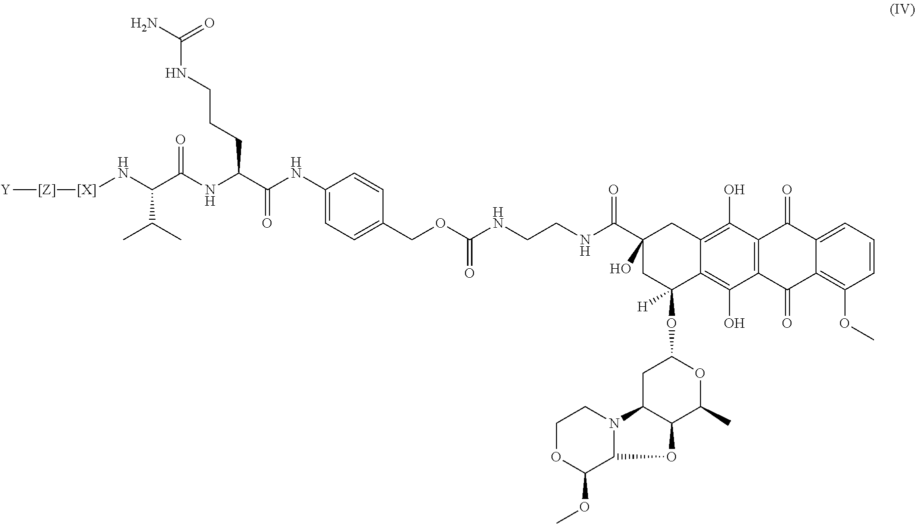

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y comprises a ROR1 specific antigen binding molecule according to the first, second or ninth aspect, or a recombinant fusion protein according to the third or fourth aspect, or a recombinant fusion protein dimer of the fifth aspect.

### DESCRIPTION OF FIGURES

FIG. 1: Design of B1 loop library: The sequence of B1 is shown with the “X” indicating amino acids within CDR1 and CDR3 which were randomised.

FIG. 2: Cell surface binding of B1 VNAR loop variants (His₆Myc tag) to A549 (ROR1 ^{hi}) lung cancer cells by flow cytometry.

FIG. 3: Cell surface binding of B1 VNAR loop variants (His₆Myc tag) to A427 (ROR1 ^{low}) lung cancer cells by flow cytometry.

FIG. 4: Sequence and loop library design of P3A1 G1. CDR1 diversity results in 448 combinations, HV2 diversity results in 768 combinations and HV4 diversity results in 24 combinations.

FIG. 5: Binding of P3A1G1 loop variants to human ROR1 by ELISA. The data is plotted as the OD signal obtained at 450 nm for a fixed concentration (5 ug/mL) of each of the loop variants.

FIG. 6: Binding of P3A1G1 loop variants and parental P3A1G1 protein to human ROR1 by ELISA.

FIG. 7: Linker mouse IgG and linker human IgG sequences used in VNAR IgG Fc fusion proteins. Engineered hlgG1 Fc fusion proteins incorporate an engineered cysteine substitution in the hlgG1 Fc sequence, for example at position S239C or S442C (EU numbering) to enable site specific labelling.

FIG. 8: Cell surface binding of B1 loop variant—hFc fusion proteins to A549 (ROR1^{hi}) lung cancer cells by flow cytometry.

FIG. 9: Analysis of ROR1 bi-paratopic VNAR-hFc fusions by SDS-PAGE (4-12% Bis Tris gel, MOPS buffer, 50 mM DTT). Lane 1 G3CP-P3A1 hFc (S239C+KIH) and lane 2 G3CPG4-P3A1 hFc (S239C+KIH)

FIG. 10: Cell surface binding of ROR1 bi-paratopic VNAR-hFc fusions to A549 (ROR1 ^{hi}) and A427 (ROR1^{low}) lung cancer cells by flow cytometry.

FIG. 11: Structures of PNU-linker payloads MA-PEG-vc-PAB-EDA-PNU159682 and MA-PEG-va-EDA-PNU159682

FIG. 12: Dose response showing binding of G3CP-hFc and G3CPG4-hFc PNU conjugates and the corresponding parental proteins to human ROR1 by ELISA

FIG. 13: Potency of G3CP-hFc PNU and G3CPG4-hFc PNU conjugates in killing the ROR1 positive PA-1 cell-line and a PA-1 cell-line with ROR1 knockout

FIG. 14: In vivo efficacy of G3CP-hFc PNU and G3CPG4-hFc PNU conjugates in the ROR1+HBCx-28 patient-derived TNBC xenograft model. Data plotted until the point when the first animal in the vehicle group reached humane tumour burden.

FIG. 15: An alignment of the sequences for B1, B1 G4, B1V15, G3CP and G3CPG4. Points of variation within the CDRs and HV regions are emphasised in underline. Note B1V15 (SEQ ID NO: 115): is not a loop library variant of B1; they have identical CDR1, HV2, HV4 and CDR3 sequences.

FIG. 16: UV analysis of B1-hFc, B1G4-hFc, G3CP-hFc and G3CPG4-hFc after incubation in PBS pH 7.4 buffer at 37° C. for 96h.

FIG. 17: Size exclusion analysis (SEC) of B1-hFc, B1G4-hFc, G3CP-hFc and G3CPG4-hFc after incubation in PBS pH 7.4 buffer at 37° C. for 96h.

FIG. 18: Potency of G3CP-hFc PNU and G3CPG4-hFc PNU conjugates in killing ROR1^{low} HEK293 cells and HEK293 cells stably transfected with human ROR1

FIG. 19: In vivo efficacy of G3CP-hFc PNU and G3CPG4-hFc PNU conjugates in the ROR1+HBCx-10 patient-derived TNBC xenograft model. Data plotted until the point when the first animal in the vehicle group reached humane tumour burden

FIG. 20: Cell surface binding of ROR1 bi-paratopic VNAR-hFc drug conjugates to A549 (ROR1^{hi}) and A427 (ROR1^{low}) lung cancer cells by flow cytometry.

FIG. 21: Potency of bi-paratopic G3CP-P3A1-hFc PNU and G3CPG4-P3A1-hFc PNU conjugates in killing the ROR1 positive PA-1 cell-line and a PA-1 cell-line with ROR1 knockout

FIG. 22: In vivo efficacy of bi-paratopic G3CP-P3A1 hFc PNU and G3CPG4-P3A1-hFc PNU conjugates in the ROR1+HBCx-28 patient-derived TNBC xenograft model.

### DETAILED DESCRIPTION

The present invention generally relates to specific antigen binding molecules. Specifically, the invention provides immunoglobulin-like shark variable novel antigen receptors (VNARs) specific for receptor tyrosine kinase-like orphan receptor 1 (ROR1) and associated fusion proteins, chimeric antigen receptors, conjugates, and nucleic acids, as well as accompanying methods. The ROR1-specifc VNAR domains are described herein as ROR1-specific antigen binding molecules.

The Novel or New antigen receptor (IgNAR) is an approximately 160 kDa homodimeric protein found in the sera of cartilaginous fish (Greenberg A. S., et al., Nature, 1995. 374(6518): p. 168-173, Dooley, H., et al, Mol. Immunol, 2003. 40(1): p. 25-33; Müller, M. R., et al., mAbs, 2012. 4(6): p. 673-685)). Each molecule consists of a single N-terminal variable domain (VNAR) and five constant domains (CNAR). The IgNAR domains are members of the immunoglobulin-superfamily. The VNAR is a tightly folded domain with structural and some sequence similarities to the immunoglobulin and T-cell receptor Variable domains and to cell adhesion molecules and is termed the VNAR by analogy to the N Variable terminal domain of the classical immunoglobulins and T Cell receptors. The VNAR shares limited sequence homology to immunoglobulins, for example 25-30% similarity between VNAR and human light chain sequences.

Kovaleva M. et al Expert Opin. Biol. Ther. 2014. 14(10): p. 1527-1539 and Zielonka S. et al mAbs 2015. 7(1): p. 15-25 provided summaries of the structural characterization and generation of the VNARs, which are hereby incorporated by reference.

The VNAR does not appear to have evolved from a classical immunoglobulin antibody ancestor. The distinct structural features of VNARs are the truncation of the sequences equivalent to the CDR2 loop present in conventional immunoglobulin variable domains and the lack of the hydrophobic VH/VL interface residues which would normally allow association with a light chain domain, which is not present in the IgNAR structure. Furthermore, unlike classical immunoglobulins some VNAR subtypes include extra cysteine residues in the CDR regions that are observed to form disulphide bridges in addition to the canonical Immunoglobulin superfamily bridge between the Cysteines in the Framework 1 and 3 regions N terminally adjacent to CDRs 1 and 3.

To date, there are three defined types of shark IgNAR known as I, II and III. These have been categorized based on the position of non-canonical cysteine residues which are under strong selective pressure and are therefore rarely replaced.

All three types have the classical immunoglobulin canonical cysteines at positions 35 and 107 (numbering as in Kabat, E. A. et al. Sequences of proteins of immunological interest. 5th ed. 1991, Bethesda: US Dept. of Health and Human Services, PHS, NIH) that stabilize the standard immunoglobulin fold, together with an invariant tryptophan at position 36. There is no defined CDR2 as such, but regions of sequence variation that compare more closely to TCR HV2 and HV4 have been defined in framework 2 and 3 respectively. Type I has germline encoded cysteine residues in framework 2 and framework 4 and an even number of additional cysteines within CDR3. Crystal structure studies of a Type I IgNAR isolated against and in complex with lysozyme enabled the contribution of these cysteine residues to be determined. Both the framework 2 and 4 cysteines form disulphide bridges with those in CDR3 forming a tightly packed structure within which the CDR3 loop is held tightly down towards the HV2 region. To date Type I IgNARs have only been identified in nurse sharks—all other elasmobranchs, including members of the same order have only Type II or variations of this type.

Type II IgNAR are defined as having a cysteine residue in CDR1 and CDR3 which form intramolecular disulphide bonds that hold these two regions in close proximity, resulting in a protruding CDR3 that is conducive to binding pockets or grooves. Type I sequences typically have longer CDR3s than type II with an average of 21 and 15 residues respectively. This is believed to be due to a strong selective pressure for two or more cysteine residues in Type I CDR3 to associate with their framework 2 and 4 counterparts. Studies into the accumulation of somatic mutations show that there are a greater number of mutations in CDR1 of type II than type I, whereas HV2 regions of Type I show greater sequence variation than Type II. This evidence correlates well with the determined positioning of these regions within the antigen binding sites.

A third IgNAR type known as Type III has been identified in neonates. This member of the IgNAR family lacks diversity within CDR3 due to the germline fusion of the D1 and D2 regions (which form CDR3) with the V-gene. Almost all known clones have a CDR3 length of 15 residues with little or no sequence diversity.

Another structural type of VNAR, termed type (IIb or IV), has only two canonical cysteine residues (in framework 1 and framework 3b regions). So far, this type has been found primarily in dogfish sharks and was also isolated from semisynthetic V-NAR libraries derived from wobbegong sharks.

The VNAR binding surface, unlike the variable domains in other natural immunoglobulins, derives from four regions of diversity: CDR1, HV2, HV4 and CDR3, joined by intervening framework sequences in the order: FW1-CDR1-FW2-HV2-FW3a-HV4-FW3b-CDR3-FW4. The combination of a lack of a natural light chain partner and lack of CDR2 make VNARs the smallest naturally occurring binding domains in the vertebrate kingdom.

The IgNAR shares some incidental features with the heavy chain only immunoglobulin (HCAb) found in camelidae (camels, dromedaries and llamas) Unlike the IgNAR the HCAb is clearly derived from the immunoglobulin family and shares significant sequence homology to standard immunogloblulins.

Importantly one key distinction of VNARs is that the molecule has not had at any point in its evolution a partner light chain, unlike classical immunoglobulins or the HCAbs. Flajnik M. F. et al PLoS Biol 2011. 9(8): el001120 and Zielonka S. et al mAbs 2015. 7(1): p. 15-25 have commented on the similarities and differences between, and the possible and distinct evolutionary origins of, the VNAR and the immunoglobulin-derived VHH single binding domain from the camelids.

Although antibodies to ROR1 have been reported in the literature, the high sequence identity between the extracellular domain of human, mouse and rat ROR1 and between human ROR1 and ROR2 family members means generating high affinity hROR1-specifc binding agents is not trivial. Additionally, the large size of antibodies compromises their ability to penetrate into solid tumours and render regions of target proteins inaccessible due to steric factors, which can be particularly acute for cell-surface proteins where oligomerisation or receptor clustering is observed.

As a result there is a need in the art for improved anti-ROR1 binding protein agents with different functional or physical characteristics or properties to antibodies and the development of therapeutics and diagnostic agents for malignancies associated with ROR1 expression. The present invention provides such agents in the form of the ROR1-specific antigen binding molecules described herein.

Without being bound by theory, the presently-described ROR1-specific antigen binding molecules are thought to bind to both human and murine ROR1. A number of variants, including G3CP, 1 E5, 1 B11, C3CP, 1G9, 1H8, G11 CP, D9CP, 1B6, 1 F10, F2CP, B6CP, 1E1 and P3A1G1 NAC6.S, P3A1G1 AE3.S, P3A1G1 NAC6, P3A1G1 AE3 and P3A1G1 NAG8 have been experimentally confirmed to bind to both hROR1 and mROR1. Furthermore, the ROR1-specific antigen binding molecules of the present invention may bind to deglycosylated forms of ROR1. Furthermore, they may not bind to a number of linear peptides associated with anti-ROR1 antibodies described in the prior art. The presently-described ROR1-specific antigen binding molecules are therefore thought to bind to distinct epitopes in the ROR1 sequence compared to these prior art anti-ROR1 antibodies.

Binding of ROR1-specific antigen binding molecules of the invention to cancer cell lines, as well as internalisation, have been demonstrated. This confirms the potential for the use of such molecules in the treatment of cancers, specifically cancers which express ROR1.

Various forms of the ROR1-specific antigen binding molecules are described, including fusion proteins of several types. Fusion proteins including an immunoglobulin Fc region are described, as well as both homo and heterodimers. Fusion of proteins to an Fc domain can improve protein solubility and stability, markedly increase plasma half-life and improve overall therapeutic effectiveness.

The present inventors have also created VNAR molecules conjugated to a variety of moieties and payloads. The present invention therefore also provides chemically conjugated VNARs. More specifically, ROR1-specific antigen binding molecules in several conjugated formats are provided.

According to a first aspect, the invention provides a receptor tyrosine kinase-like orphan receptor 1 (ROR1) specific antigen binding molecule comprising an amino acid sequence represented by the formula (1):

FW1-CDR1-FW2-HV2-FW3a-HV4-FW3b-CDR3-FW4 (I)
wherein
   CDR3 is a CDR sequence having an amino acid sequence selected from the group consisting of YPWGAGAPYNVQWY (SEQ ID NO: 23), YPWGAGAPYLVQWY (SEQ ID NO: 20), YPWGAGAPWNVQWY (SEQ ID NO: 24), YPSGAGAPRPVQWY (SEQ ID NO:11), YPWGAGAPCLVQWY (SEQ ID NO: 12), YPWGAGAPRLVQWY (SEQ ID NO:13), YPWGAGAPRQVQWY (SEQ ID NO: 14), YPWGAGAPRSVQWY (SEQ ID NO: 15), YPWGAGAPSLVQWY (SEQ ID NO: 16), YPWGAGAPSNVQWY (SEQ ID NO: 17), YPWGAGAPSQVQWY (SEQ ID NO: 18), YPWGAGAPSSVQWY (SEQ ID NO: 19), YPWGAGAPWQVQWY (SEQ ID NO: 21), YPWGAGAPWSVQWY (SEQ ID NO: 22), and YPWGAGAPWLVQWY (SEQ ID NO: 10);
   CDR1 is a CDR sequence having an amino acid sequence selected from the group consisting of GANYGLAA (SEQ ID NO: 1), DANYGLAA (SEQ ID NO: 5), GANYDLSA (SEQ ID NO: 2), GANYGLSA (SEQ ID NO: 3), and GANYDLAA (SEQ ID NO: 4) FW1 is a framework region;
   FW2 is a framework region;
   HV2 is a hypervariable sequence having an amino acid sequence selected from the group consisting of SSNQERISIS (SEQ ID NO: 6) and SSNKERISIS (SEQ ID NO: 7);
   FW3a is a framework region;
   HV4 is a hypervariable sequence having an amino acid sequence selected from the group consisting of NKRTM (SEQ ID NO: 8) and NKGTM (SEQ ID NO: 9);
   FW3b is a framework region;
   FW4 is a framework region;
wherein if CDR3 is YPWGAGAPWLVQWY (SEQ ID NO: 10) then CDR1 is selected from the group consisting of DANYGLAA (SEQ ID NO: 5), GANYGLSA (SEQ ID NO: 3) and GANYDLAA (SEQ ID NO: 4).

In one embodiment of the ROR1-specific antigen binding molecule:
CDR3 is a CDR sequence having an amino acid sequence selected from the group consisting of YPWGAGAPYNVQWY (SEQ ID NO: 23), YPWGAGAPYLVQWY (SEQ ID NO: 20), YPWGAGAPWNVQWY (SEQ ID NO: 24), YPSGAGAPRPVQWY (SEQ ID NO:11), YPWGAGAPCLVQWY (SEQ ID NO: 12), YPWGAGAPRLVQWY (SEQ ID NO:13), YPWGAGAPRQVQWY (SEQ ID NO: 14), YPWGAGAPRSVQWY (SEQ ID NO: 15), YPWGAGAPSLVQWY (SEQ ID NO: 16), YPWGAGAPSNVQWY (SEQ ID NO: 17), YPWGAGAPSSVQWY (SEQ ID NO: 19), YPWGAGAPWQVQWY (SEQ ID NO: 21), YPWGAGAPWSVQWY (SEQ ID NO: 22), and YPWGAGAPWLVQWY (SEQ ID NO: 10).

If CDR3 is not YPWGAGAPWLVQWY (SEQ ID NO: 10) then CDR1 may be a CDR sequence having an amino acid sequence selected from the group consisting of GANYGLAA (SEQ ID NO: 1), DANYGLAA (SEQ ID NO: 5), GANYDLSA (SEQ ID NO: 2), GANYGLSA (SEQ ID NO: 3), and GANYDLAA (SEQ ID NO: 4). Accordingly, the ROR1 specific antigen binding molecule may be defined as comprising an amino acid sequence represented by the formula (I):

FW1-CDR1-FW2-HV2-FW3a-HV4-FW3b-CDR3-FW4 (I)
wherein
   CDR3 is a CDR sequence having an amino acid sequence selected from the group consisting of YPWGAGAPYNVQWY (SEQ ID NO: 23), YPWGAGAPYLVQWY (SEQ ID NO: 20), YPWGAGAPWNVQWY (SEQ ID NO: 24), YPSGAGAPRPVQWY (SEQ ID NO:11), YPWGAGAPCLVQWY (SEQ ID NO: 12), YPWGAGAPRLVQWY (SEQ ID NO:13), YPWGAGAPRQVQWY (SEQ ID NO: 14), YPWGAGAPRSVQWY (SEQ ID NO: 15), YPWGAGAPSLVQWY (SEQ ID NO: 16), YPWGAGAPSNVQWY (SEQ ID NO: 17), YPWGAGAPSQVQWY (SEQ ID NO: 18), YPWGAGAPSSVQWY (SEQ ID NO: 19), YPWGAGAPWQVQWY (SEQ ID NO: 21), and YPWGAGAPWSVQWY (SEQ ID NO: 22);
   CDR1 is a CDR sequence having an amino acid sequence selected from the group consisting of GANYGLAA (SEQ ID NO: 1), DANYGLAA (SEQ ID NO: 5), GANYDLSA (SEQ ID NO: 2), GANYGLSA (SEQ ID NO: 3), and GANYDLAA (SEQ ID NO: 4);
   FW1 is a framework region;
   FW2 is a framework region;
   HV2 is a hypervariable sequence having an amino acid sequence selected from the group consisting of SSNQERISIS (SEQ ID NO: 6) and SSNKERISIS (SEQ ID NO: 7);
   FW3a is a framework region;
   HV4 is a hypervariable sequence having an amino acid sequence selected from the group consisting of NKRTM (SEQ ID NO: 8) and NKGTM (SEQ ID NO: 9);
   FW3b is a framework region; and
   FW4 is a framework region.

In one embodiment of the ROR1-specific antigen binding molecule:
CDR3 is a CDR sequence having an amino acid sequence selected from the group consisting of YPWGAGAPYNVQWY (SEQ ID NO: 23), YPWGAGAPYLVQWY (SEQ ID NO: 20) and YPWGAGAPWNVQWY (SEQ ID NO: 24), and/or
CDR1 is a CDR sequence having an amino acid sequence selected from the group consisting of GANYGLAA (SEQ ID NO: 1) and DANYGLAA (SEQ ID NO: 5).

In one embodiment of the ROR1-specific antigen binding molecule:
CDR3 is a CDR sequence having an amino acid sequence according to YPWGAGAPYNVQWY (SEQ ID NO: 23).

In one embodiment of the ROR1-specific antigen binding molecule:
CDR3 is a CDR sequence having an amino acid sequence according to YPWGAGAPYNVQWY (SEQ ID NO: 23);
CDR1 is a CDR sequence having an amino acid sequence according to GANYGLAA (SEQ ID NO: 1);
HV2 is a hypervariable sequence having an amino acid sequence according to SSNQERISIS (SEQ ID NO: 6); and
HV4 is a hypervariable sequence having an amino acid sequence according to NKRTM (SEQ ID NO: 8).

In one embodiment of the ROR1-specific antigen binding molecule:
CDR3 is a CDR sequence having an amino acid sequence according to YPWGAGAPYNVQWY (SEQ ID NO: 23);
CDR1 is a CDR sequence having an amino acid sequence according to DANYGLAA (SEQ ID NO: 5);
HV2 is a hypervariable sequence having an amino acid sequence according to SSNKERISIS (SEQ ID NO: 7); and
HV4 is a hypervariable sequence having an amino acid sequence according to NKGTM (SEQ ID NO: 9).

In one embodiment of the ROR1-specific antigen binding molecule:
CDR3 is a CDR sequence having an amino acid sequence according to YPWGAGAPWLVQWY (SEQ ID NO: 10);
CDR1 is a CDR sequence having an amino acid sequence according to DANYGLAA (SEQ ID NO: 5);
HV2 is a hypervariable sequence having an amino acid sequence according to SSNKERISIS (SEQ ID NO: 7); and
HV4 is a hypervariable sequence having an amino acid sequence according to NKGTM (SEQ ID NO: 9).

In preferred embodiments the ROR1-specific antigen binding molecule comprises an amino acid sequence selected from the group consisting of:

(SEQ ID NO: 50)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVN referred to herein as G3CP;
(SEQ ID NO: 51)
TRVDQSPSSLSASVGDRVTITCVLTDANYGLAATYWYRKNPGSSNKERIS
ISGRYSESVNKGTMSFTLTISSLQPEDSATYYCRAYPWGAGAPWLVQWYD
GAGTKVEIK referred to herein as B1G4;
(SEQ ID NO: 52)
ASVNQTPRTATKETGESLTINCVVTGANYDLSATYWYRKNPGSSNQERIS
ISGRYVESVNKRTMSFSLRIKDLTVADSATYYCKAYPSGAGAPRPVQWYD
GAGTVLTVN referred to herein as 1E2;
(SEQ ID NO: 53)
ASVNQTPRTATKETGESLTINCVVTGANYGLAATYWYRKNPGSSNQERIS
ISGRYVESVNKRTMSFSLRIKDLTVADSATYYCKAYPWGAGAPCLVQWYD
GAGTVLTVN referred to herein as 1E5;
(SEQ ID NO: 54)
ASVNQTPRTATKETGESLTINCVVTGANYGLAATYWYRKNPGSSNQERIS
ISGRYVESVNKRTMSFSLRIKDLTVADSATYYCKAYPWGAGAPRLVQWYD
GAGTVLTVN referred to herein as 1B11;
(SEQ ID NO: 55)
ASVNQTPRTATKETGESLTINCVVTGANYGLAATYWYRKNPGSSNQERIS
ISGRYVESVNKRTMSFSLRIKDLTVADSATYYCKAYPWGAGAPRQVQWYD
GAGTVLTVN referred to herein as C3CP;
(SEQ ID NO: 56)
ASVNQTPRTATKETGESLTINCVVTGANYGLSATYWYRKNPGSSNQERIS
ISGRYVESVNKRTMSFSLRIKDLTVADSATYYCKAYPWGAGAPRSVQWYD
GAGTVLTVN referred to herein as 2G5;
(SEQ ID NO: 57)
ASVNQTPRTATKETGESLTINCVVTGANYGLAATYWYRKNPGSSNQERIS
ISGRYVESVNKRTMSFSLRIKDLTVADSATYYCKAYPWGAGAPRSVQWYD
GAGTVLTVN referred to herein as 1G12;
(SEQ ID NO: 58)
ASVNQTPRTATKETGESLTINCVVTGANYGLAATYWYRKNPGSSNQERIS
ISGRYVESVNKRTMSFSLRIKDLTVADSATYYCKAYPWGAGAPSLVQWYD
GAGTVLTVN referred to herein as G5CP;
(SEQ ID NO: 59)
ASVNQTPRTATKETGESLTINCVVTGANYGLSATYWYRKNPGSSNQERIS
ISGRYVESVNKRTMSFSLRIKDLTVADSATYYCKAYPWGAGAPSNVQWYD
GAGTVLTVN referred to herein as 2F4;
(SEQ ID NO: 60)
ASVNQTPRTATKETGESLTINCVVTGANYGLAATYWYRKNPGSSNQERIS
ISGRYVESVNKRTMSFSLRIKDLTVADSATYYCKAYPWGAGAPSQVQWYD
GAGTVLTVN referred to herein as 1G9;
(SEQ ID NO: 61)
ASVNQTPRTATKETGESLTINCVVTGANYGLAATYWYRKNPGSSNQERIS
ISGRYVESVNKRTMSFSLRIKDLTVADSATYYCKAYPWGAGAPSSVQWYD
GAGTVLTVN referred to herein as 1H8;
(SEQ ID NO: 62)
ASVNQTPRTATKETGESLTINCVVTGANYGLSATYWYRKNPGSSNQERIS
ISGRYVESVNKRTMSFSLRIKDLTVADSATYYCKAYPWGAGAPWLVQWYD
GAGTVLTVN referred to herein as G11CP;
(SEQ ID NO: 63)
ASVNQTPRTATKETGESLTINCVVTGANYDLAATYWYRKNPGSSNQERIS
ISGRYVESVNKRTMSFSLRIKDLTVADSATYYCKAYPWGAGAPWLVQWYD
GAGTVLTVN referred to herein as D9CP;
(SEQ ID NO: 64)
ASVNQTPRTATKETGESLTINCVVTGANYGLAATYWYRKNPGSSNQERIS
ISGRYVESVNKRTMSFSLRIKDLTVADSATYYCKAYPWGAGAPWNVQWYD
GAGTVLTVN referred to herein as 1B6;
(SEQ ID NO: 65)
ASVNQTPRTATKETGESLTINCVVTGANYGLAATYWYRKNPGSSNQERIS
ISGRYVESVNKRTMSFSLRIKDLTVADSATYYCKAYPWGAGAPWQVQWYD
GAGTVLTVN referred to herein as 1F10;
(SEQ ID NO: 66)
ASVNQTPRTATKETGESLTINCVVTGANYGLSATYWYRKNPGSSNQERIS
ISGRYVESVNKRTMSFSLRIKDLTVADSATYYCKAYPWGAGAPWQVQWYD
GAGTVLTVN referred to herein as E6CP;
(SEQ ID NO: 67)
ASVNQTPRTATKETGESLTINCVVTGANYDLAATYWYRKNPGSSNQERIS
ISGRYVESVNKRTMSFSLRIKDLTVADSATYYCKAYPWGAGAPWQVQWYD
GAGTVLTVN referred to herein as F2CP;
(SEQ ID NO: 68)
ASVNQTPRTATKETGESLTINCVVTGANYDLAATYWYRKNPGSSNQERIS
ISGRYVESVNKRTMSFSLRIKDLTVADSATYYCKAYPWGAGAPWSVQWYD
GAGTVLTVN referred to herein as B6CP;
(SEQ ID NO: 69)
ASVNQTPRTATKETGESLTINCVVTGANYGLAATYWYRKNPGSSNQERIS
ISGRYVESVNKRTMSFSLRIKDLTVADSATYYCKAYPWGAGAPWSVQWYD
GAGTVLTVN referred to herein as 1G1;
and
(SEQ ID NO: 70)
ASVNQTPRTATKETGESLTINCVVTGANYGLSATYWYRKNPGSSNQERIS
ISGRYVESVNKRTMSFSLRIKDLTVADSATYYCKAYPWGAGAPWSVQWYD
GAGTVLTVN referred to herein as A10CP;

or a functional variant having CDR1, HV2, HV4 and CDR2 sequences according to any thereof and having FW1, FW2, FW3a, FW3b and FW4 sequences having a combined sequence identity of at least 45% to the combined FW1, FW2, FW3a, FW3b and FW4 sequences of any thereof.

In a particularly preferred embodiment, the ROR1-specific antigen binding molecule may comprise an amino acid sequence according to

(SEQ ID NO: 50)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVN.

The ROR1-specific antigen binding molecule may comprise an amino acid sequence according to ASVNQTPRTATKETGESLTINCVVTGANYGLAATYWYRKNPGSSNQERISISGRYVESVNKRTMSFSL RIKDLTVADSATYYCKAYPWGAGAPYNVQWYDGAGTVLTVN (SEQ ID NO: 50) or a functional variant thereof having CDR1, HV2, HV4 and CDR2 sequences according to SEQ ID NO: 50 and having FW1, FW2, FW3a, FW3b and FW4 sequences having a combined sequence identity of at least 45% to the combined FW1, FW2, FW3a, FW3b and FW4 sequences of SEQ ID NO: 50.

Particular advantages associated with SEQ ID NO: 50 (“G3CP”) and functional variants thereof include increased expression yields and hydrophilicity and increased ease of analysis, purification and monomericity in non-optimised aqueous buffer systems for these proteins. Without being bound by theory, these advantages may be particularly evident in VNAR-hFc fusion proteins comprising the G3CP sequence or functional variants thereof. The G3CP sequence and functional variants thereof may therefore provide improved manufacturing and/or handling properties. Furthermore, G3CP-hFc shows excellent in vivo efficacy in a patient-derived xenograft model of Triple Negative Breast Cancer (TNBC) when conjugated to a cytotoxic anthracycline (PNU) derivative. The effect of G3CP-hFc is surprisingly improved over even B1-hFc which itself shows excellent in vivo efficacy.

In a particularly preferred embodiment, the ROR1-specific antigen binding molecule may comprise an amino acid sequence according to

(SEQ ID NO: 51)
TRVDQSPSSLSASVGDRVTITCVLTDANYGLAATYWYRKNPGSSNKERIS
ISGRYSESVNKGTMSFTLTISSLQPEDSATYYCRAYPWGAGAPWLVQWYD
GAGTKVEIK.

Particular advantages associated with SEQ ID NO: 51 (“B1G4”) and functional variants thereof include increased expression yields and monomericity in aqueous buffer systems for fusion proteins comprising the B1G4 sequence or functional variants thereof, such as VNAR-hFc fusion proteins. The B1G4 sequence and functional variants thereof may therefore provide fusion proteins with improved manufacturing and/or handling properties.

The ROR1-specific antigen binding molecule may comprise an amino acid sequence according to TRVDQSPSSLSASVGDRVTITCVLTDANYGLAATYWYRKNPGSSNKERISISGRYSESVNKGTMSFTL TISSLQPEDSATYYCRAYPWGAGAPWLVQWYDGAGTKVEIK (SEQ ID NO: 51) or a functional variant thereof having CDR1, HV2, HV4 and CDR2 sequences according to SEQ ID NO: 51 and having FW1, FW2, FW3a, FW3b and FW4 sequences having a combined sequence identity of at least 45% to the combined FW1, FW2, FW3a, FW3b and FW4 sequences of SEQ ID NO: 51.

In preferred embodiments the ROR1-specific antigen binding molecule comprises an amino acid sequence selected from the group consisting of:

(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK referred to herein as G3CP G4;
(SEQ ID NO: 72)
ASVTQSPRSASKETGESLTITCRVTGANYGLAATYWYRKNPGSSNQERIS
ISGRYSESVNKRTMSFSLRISSLTVEDSATYYCKAYPWGAGAPYNVQWYD
GQGTKLEVK referred to herein as G3CP V15;
(SEQ ID NO: 73)
TRVDQSPSSLSASVGDRVTITCVLTDANYGLAATYWYRKNPGSSNKERIS
ISGRYSESVNKGTMSFTLTISSLQPEDSATYYCRAYPWGAGAPSSVQWYD
GAGTKVEIK referred to herein as 1H8 G4;
(SEQ ID NO: 74)
ASVTQSPRSASKETGESLTITCRVTGANYGLAATYWYRKNPGSSNQERIS
ISGRYSESVNKRTMSFSLRISSLTVEDSATYYCKAYPWGAGAPSSVQWYD
GQGTKLEVK referred to herein as 1H8 V15;
(SEQ ID NO: 75)
TRVDQSPSSLSASVGDRVTITCVLTDANYGLAATYWYRKNPGSSNKERIS
ISGRYSESVNKGTMSFTLTISSLQPEDSATYYCRAYPWGAGAPRQVQWYD
GAGTKVEIK referred to herein as C3CP G4;
and
(SEQ ID NO: 76)
ASVTQSPRSASKETGESLTITCRVTGANYGLAATYWYRKNPGSSNQERIS
ISGRYSESVNKRTMSFSLRISSLTVEDSATYYCKAYPWGAGAPRQVQWYD
GQGTKLEVK referred to herein as C3CPV15;

or a functional variant having CDR1, HV2, HV4 and CDR2 sequences according to any thereof and having FW1, FW2, FW3a, FW3b and FW4 sequences having a combined sequence identity of at least 45% to the combined FW1, FW2, FW3a, FW3b and FW4 sequences of any thereof.

In a particularly preferred embodiment, the ROR1-specific antigen binding molecule may comprise an amino acid sequence according to

(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK.

The ROR1-specific antigen binding molecule may comprise an amino acid sequence according to TRVDQSPSSLSASVGDRVTITCVLTDANYGLAATYWYRKNPGSSNKERISISGRYSESVNKGTMSFTL TISSLQPEDSATYYCRAYPWGAGAPYNVQWYDGAGTKVEIK (SEQ ID NO: 71) or a functional variant thereof having CDR1, HV2, HV4 and CDR2 sequences according to SEQ ID NO: 71 and having FW1, FW2, FW3a, FW3b and FW4 sequences having a combined sequence identity of at least 45% to the combined FW1, FW2, FW3a, FW3b and FW4 sequences of SEQ ID NO: 71.

Particular advantages associated with SEQ ID NO: 71 (“G3CP G4”) and functional variants thereof include increased expression yields and hydrophilicity and increased ease of analysis, purification and monomericity in non-optimised aqueous buffer systems for these proteins. Without being bound by theory, these advantages may be particularly evident in VNAR-hFc fusion proteins comprising the G3CP G4 sequence or functional variants thereof. The G3CP G4 sequence and functional variants thereof may therefore provide improved manufacturing and/or handling properties. Furthermore, G3CPG4-hFc shows excellent in vivo efficacy in a patient-derived xenograft model of Triple Negative Breast Cancer (TNBC) when conjugated to a cytotoxic anthracycline (PNU) derivative. The effect of G3CPG4-hFc is surprisingly improved over even B1-hFc which itself shows excellent in vivo efficacy.

The sequences of G3CP and G3CPG4 have in common two single amino acid changes relative to the sequence of B1. These are both within CDR3 and are the substitution:
1. Of a W residue with a Y residue, and
2. Of an L residue with a N residue.

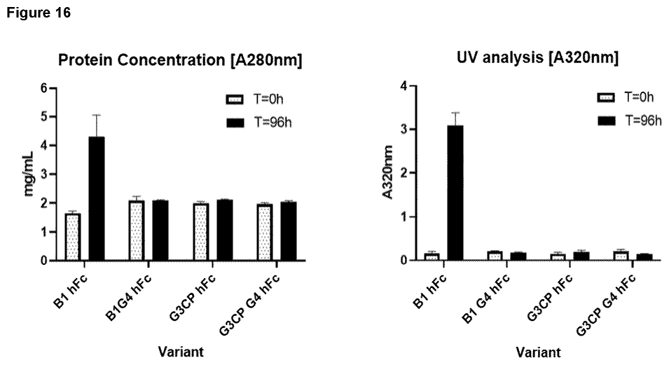

Compared to G3CP, G3CPG4 has a further single amino acid change in each of CDR1, HV2 and HV4 relative to B1 (which also appear in B1 G4) and changes to humanise the framework regions (some of which also appear in B1V15, SEQ ID NO: 115, as shown in FIG. 15—B1V15 has the same CDR1, HV2, HV4 and CDR3 sequences as B1 i.e. it is not a loop library variant; the changes to B1V15 relative to B1 are in the framework regions only).

Without being bound by theory, any improvements over B1 shown by both G3CP and G3CPG4 which are not shown by B1 G4 or B1 V15 are thought to result from one or both of the two mutations they share in CDR3. Accordingly, advantages of G3CP and G3CPG4 are thought to derive from a CDR3 comprising the sequence YPWGAGAPYNVQWY (SEQ ID NO: 23).

Without being bound by theory, the surprising advantages associated with YPWGAGAPYNVQWY (SEQ ID NO: 23) may represent a synergistic effect of both the W to Y and the L to N substitutions. Alternatively, the surprising advantages may derive primarily from the W to Y substitution thus being shared by YPWGAGAPYLVQWY (SEQ ID NO: 20). 1B6, which has the L to N mutation and a CDR3 sequence of YPWGAGAPWNVQWY (SEQ ID NO: 24), has a lower elution volume than B1, therefore the L to N mutation in CDR3 does lead to improved manufacturing and/or handling properties.

According to a second aspect, the invention provides a receptor tyrosine kinase-like orphan receptor 1 (ROR1) specific antigen binding molecule comprising an amino acid sequence represented by the formula (I):

FW1-CDR1-FW2-HV2-FW3a-HV4-FW3b-CDR3-FW4 (I)
wherein
   CDR1 is a CDR sequence having an amino acid sequence selected from the group consisting of GTRYGLYS (SEQ ID NO: 25), GTRYGLYSS (SEQ ID NO: 26), DTRYALYS (SEQ ID NO: 27), DTRYALYSS (SEQ ID NO: 28), GTKYGLYA (SEQ ID NO: 29) and GTKYGLYAS (SEQ ID NO: 30);
   FW1 is a framework region;
   FW2 is a framework region;
   HV2 is a hypervariable sequence having an amino acid sequence selected from the group consisting of SSDEERISIS (SEQ ID NO: 31), STDEERISIG (SEQ ID NO: 32), SPNKDRMIIG (SEQ ID NO: 33), and STDKERIIIG (SEQ ID NO: 34);
   FW3a is a framework region;
   HV4 is a hypervariable sequence having an amino acid sequence selected from the group consisting of NKGTK (SEQ ID NO: 35), NKGSK (SEQ ID NO: 36), NNGTK (SEQ ID NO: 37), and NNRSK (SEQ ID NO: 38);
   FW3b is a framework region;
   CDR3 is a CDR sequence having an amino acid sequence according to REARHPWLRQWY (SEQ ID NO: 39);
   FW4 is a framework region.

In preferred embodiments the ROR1-specific antigen binding molecule comprises an amino acid sequence selected from the group consisting of:

(SEQ ID NO: 77)
TRVDQSPSSLSASVGDRVTITCVLTGTRYGLYSTYWYRKNPGSSDEERIS
ISGRYSESVNKGTKSFTLTISSLQPEDSATYYCRAREARHPWLRQWYDGA
GTKVEIK referred to herein as P3A1 G1 AE3;
(SEQ ID NO: 78)
TRVDQSPSSLSASVGDRVTITCVLTGTRYGLYSSTYWYRKNPGSSDEERI
SISGRYSESVNKGTKSFTLTISSLQPEDSATYYCRAREARHPWLRQWYDG
AGTKVEIK referred to herein as P3A1 G1 AE3.S;
(SEQ ID NO: 79)
TRVDQSPSSLSASVGDRVTITCVLTDTRYALYSTYWYRKNPGSTDEERIS
IGGRYSESVNKGSKSFTLTISSLQPEDSATYYCRAREARHPWLRQWYDGA
GTKVEIK referred to herein as P3A1 G1 NAC6;
(SEQ ID NO: 80)
TRVDQSPSSLSASVGDRVTITCVLTDTRYALYSSTYWYRKNPGSTDEERI
SIGGRYSESVNKGSKSFTLTISSLQPEDSATYYCRAREARHPWLRQWYDG
AGTKVEIK referred to herein as P3A1 G1 NAC6.S;
(SEQ ID NO: 81)
TRVDQSPSSLSASVGDRVTITCVLTGTKYGLYATYWYRKNPGSPNKDRMI
IGGRYSESVNNGTKSFTLTISSLQPEDSATYYCRAREARHPWLRQWYDGA
GTKVEIK referred to herein as P3A1 G1 NAG8;
(SEQ ID NO: 82)
TRVDQSPSSLSASVGDRVTITCVLTGTKYGLYASTYWYRKNPGSPNKDRM
IIGGRYSESVNNGTKSFTLTISSLQPEDSATYYCRAREARHPWLRQWYDG
AGTKVEIK referred to herein as P3A1 G1 NAG8.S;
(SEQ ID NO: 83)
TRVDQSPSSLSASVGDRVTITCVLTGTKYGLYASTYWYRKNPGSTDKERI
IIGGRYSESVNNRSKSFTLTISSLQPEDSATYYCRAREARHPWLRQWYDG
AGTKVEIK referred to herein as P3A1 G1 AF7.S;

or a functional variant having CDR1, HV2, HV4 and CDR2 sequences according to any thereof and having FW1, FW2, FW3a, FW3b and FW4 sequences having a combined sequence identity of at least 45% to the combined FW1, FW2, FW3a, FW3b and FW4 sequences of any thereof.

Particular advantages associated with affinity matured variants of P3A1G1 and functional variants thereof include improved binding to hROR1-Fc compared to parental P3A1G1 as illustrated by the Examples.

The ROR1-specific antigen binding molecule may comprise the CDR and HV sequences of a clone set out in Table 1 below. In preferred embodiments of the first and/or second aspect of the invention, the ROR1-specific antigen binding molecule has the combined sequence of any of the clones set out in Table 1 below.

TABLE 1
B1 loop variants

| Clone | | | | | | | | | |
|---|---|---|---|---|---|---|---|---|---|
| Name | FW1 | CDR1 | FW2 | HV2 | FW3a | HV4 | FW3b | CDR3 | FW4 |
| B1 | ASVNQTPRT | GANYGLAA | TYWYRKNP | SSNQERI | GRYVESV | NKRTM | SFSLRIKDLTVADSA | YPWGAGAPWLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 10) | ID NO: |
| | (SEQ ID | | | | | | | | 48) |
| | NO: 40) | | | | | | | | |
| B1G4 | ASVNQTPRT | DANYGLAA | TYWYRKNP | SSNKERIS | GRYSESV | NKGTM | SFTLTISSLQPEDSA | YPWGAGAPWLV | DGAGTKVE |
| | ATKETGESL | (SEQ ID | G (SEQ ID | IS (SEQ ID | (SEQ ID | (SEQ ID | TYYCRA (SEQ ID | QWY (SEQ | IK (SEQ |
| | TINCVVT | NO: 5) | NO: 43) | NO: 7) | NO: 45) | NO: 9) | NO: 46) | NO: 10) | ID NO: |
| | (SEQ ID | | | | | | | | 49) |
| | NO: 40) | | | | | | | | |
| 1E2 | ASVNQTPRT | GANYDLSA | TYWYRKNP | SSNQERI | GRYVESV | NKRTM | SFSLRIKDLTVADSA | YPSGAGAPRPV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 2) | NO: 43) | ID NO: 6) | NO: 44) | NO: 8) | NO: 84) | NO: 11) | ID NO: |
| | (SEQ ID | | | | | | | | 48) |
| | NO: 40) | | | | | | | | |
| 1E5 | ASVNQTPRT | GANYGLAA | TYWYRKNP | SSNQERI | GRYVESV | NKRTM | SFSLRIKDLTVADSA | YPWGAGAPCLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 12) | ID NO: |
| | (SEQ ID | | | | | | | | 48) |
| | NO: 40) | | | | | | | | |
| 1B11 | ASVNQTPRT | GANYGLAA | TYWYRKNP | SSNQERI | GRYVESV | NKRTM | SFSLRIKDLTVADSA | YPWGAGAPRLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 13) | ID NO: |
| | (SEQ ID | | | | | | | | 48) |
| | NO: 40) | | | | | | | | |
| C3CP | ASVNQTPRT | GANYGLAA | TYWYRKNP | SSNQERI | GRYVESV | NKRTM | SFSLRIKDLTVADSA | YPWGAGAPRQV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 14) | ID NO: |
| | (SEQ ID | | | | | | | | 48) |
| | NO: 40) | | | | | | | | |
| 2G5 | ASVNQTPRT | GANYGLSA | TYWYRKNP | SSNQERI | GRYVESV | NKRTM | SFSLRIKDLTVADSA | YPWGAGAPRSV | DGAGTVLT |
| | ATKETGESL | (SEQ ID | G (SEQ ID | SIS (SEQ | (SEQ ID | (SEQ ID | TYYCKA (SEQ | QWY (SEQ ID | VN (SEQ |
| | TINCVVT | NO: 3) | NO: 43) | ID NO: 6) | NO: 44) | NO: 8) | NO: 84) | NO: 15) | ID NO: |
| | (SEQ ID | | | | | | | | 48) |
| | NO: 40) | | | | | | | | |
| 1G12 | ASVNQTPRT | GANYGLAA | TYWYRKNP | SSNQERI | GRYVESV | NKRTM | SFSLRIKDLTVADSA | YPWGAGAPRSV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 15) | ID NO: |
| | (SEQ ID | | | | | | | | 48) |
| | NO: 40) | | | | | | | | |
| G5CP | ASVNQTPRT | GANYGLAA | TYWYRKNP | SSNQERI | GRYVESV | NKRTM | SFSLRIKDLTVADSA | YPWGAGAPSLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 16) | ID NO: |
| | (SEQ ID | | | | | | | | 48) |
| | NO: 40) | | | | | | | | |
| 2F4 | ASVNQTPRT | GANYGLSA | TYWYRKNP | SSNQERI | GRYVESV | NKRTM | SFSLRIKDLTVADSA | YPWGAGAPSNV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 3) | NO: 43) | ID NO: 6) | NO: 44) | NO: 8) | NO: 84) | NO: 17) | ID NO: |
| | (SEQ ID | | | | | | | | 48) |
| | NO: 40) | | | | | | | | |
| 1G9 | ASVNQTPRT | GANYGLAA | TYWYRKNP | SSNQERI | GRYVESV | NKRTM | SFSLRIKDLTVADSA | YPWGAGAPSQV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 18) | ID NO: |
| | (SEQ ID | | | | | | | | 48) |
| | NO: 40) | | | | | | | | |
| 1H8 | ASVNQTPRT | GANYGLAA | TYWYRKNP | SSNQERI | GRYVESV | NKRTM | SFSLRIKDLTVADSA | YPWGAGAPSSV | DGAGTVLT |
| | ATKETGESL | (SEQ ID | G (SEQ ID | SIS (SEQ | (SEQ ID | (SEQ ID | TYYCKA (SEQ | QWY (SEQ ID | VN (SEQ |
| | TINCVVT | NO: 1) | NO: 43) | ID NO: 6) | NO: 44) | NO: 8) | NO: 84) | NO: 19) | ID NO: |
| | (SEQ ID | | | | | | | | 48) |
| | NO: 40) | | | | | | | | |
| G11CP | ASVNQTPRT | GANYGLSA | TYWYRKNP | SSNQERI | GRYVESV | NKRTM | SFSLRIKDLTVADSA | YPWGAGAPWLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 3) | NO: 43) | ID NO: 6) | NO: 44) | NO: 8) | NO: 84) | NO: 10) | ID NO: |
| | (SEQ ID | | | | | | | | 48) |
| | NO: 40) | | | | | | | | |
| D9CP | ASVNQTPRT | GANYDLAA | TYWYRKNP | SSNQERI | GRYVESV | NKRTM | SFSLRIKDLTVADSA | YPWGAGAPWLV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 4) | NO: 43) | ID NO: 6) | NO: 44) | NO: 8) | NO: 84) | NO: 10) | ID NO: |
| | (SEQ ID | | | | | | | | 48) |
| | NO: 40) | | | | | | | | |
| 1B6 | ASVNQTPRT | GANYGLAA | TYWYRKNP | SSNQERI | GRYVESV | NKRTM | SFSLRIKDLTVADSA | YPWGAGAPWNV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 24) | ID NO: |
| | (SEQ ID | | | | | | | | 48) |
| | NO: 40) | | | | | | | | |
| 1F10 | ASVNQTPRT | GANYGLAA | TYWYRKNP | SSNQERI | GRYVESV | NKRTM | SFSLRIKDLTVADSA | YPWGAGAPWQV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 21) | ID NO: |
| | (SEQ ID | | | | | | | | 48) |
| | NO: 40) | | | | | | | | |
| E6CP | ASVNQTPRT | GANYGLSA | TYWYRKNP | SSNQERI | GRYVESV | NKRTM | SFSLRIKDLTVADSA | YPWGAGAPWQV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 3) | NO: 43) | ID NO: 6) | NO: 44) | NO: 8) | NO: 84) | NO: 21) | ID NO: |
| | (SEQ ID | | | | | | | | 48) |
| | NO: 40) | | | | | | | | |
| F2CP | ASVNQTPRT | GANYDLAA | TYWYRKNP | SSNQERI | GRYVESV | NKRTM | SFSLRIKDLTVADSA | YPWGAGAPWQV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 4) | NO: 43) | ID NO: 6) | NO: 44) | NO: 8) | NO: 84) | NO: 21) | ID NO: |
| | (SEQ ID | | | | | | | | 48) |
| | NO: 40) | | | | | | | | |
| B6CP | ASVNQTPRT | GANYDLAA | TYWYRKNP | SSNQERI | GRYVESV | NKRTM | SFSLRIKDLTVADSA | YPWGAGAPWSV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 4) | NO: 43) | ID NO: 6) | NO: 44) | NO: 8) | NO: 84) | NO: 22) | ID NO: |
| | (SEQ ID | | | | | | | | 48) |
| | NO: 40) | | | | | | | | |
| 1G1 | ASVNQTPRT | GANYGLAA | TYWYRKNP | SSNQERI | GRYVESV | NKRTM | SFSLRIKDLTVADSA | YPWGAGAPWSV | DGAGTVLT |
| | ATKETGESL | (SEQ ID | G (SEQ ID | SIS (SEQ | (SEQ ID | (SEQ ID | TYYCKA (SEQ ID | QWY (SEQ | VN (SEQ |
| | TINCVVT | NO: 1) | NO: 43) | ID NO: 6) | NO: 44) | NO: 8) | NO: 84) | NO: 22) | ID NO: |
| | (SEQ ID | | | | | | | | 48) |
| | NO: 40) | | | | | | | | |
| A10CP | ASVNQTPRT | GANYGLSA | TYWYRKNP | SSNQERI | GRYVESV | NKRTM | SFSLRIKDLTVADSA | YPWGAGAPWSV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 3) | NO: 43) | ID NO: 6) | NO: 44) | NO: 8) | NO: 84) | NO: 22) | ID NO: |
| | (SEQ ID | | | | | | | | 48) |
| | NO: 40) | | | | | | | | |
| G3CP | ASVNQTPRT | GANYGLAA | TYWYRKNP | SSNQERI | GRYVESV | NKRTM | SFSLRIKDLTVADSA | YPWGAGAPYNV | DGAGTVLT |
| | ATKETGESL | (SEQ ID | G (SEQ ID | SIS (SEQ | (SEQ ID | (SEQ ID | TYYCKA (SEQ ID | QWY (SEQ ID | VN (SEQ |
| | TINCVVT | NO: 1) | NO: 43) | ID NO: 6) | NO: 44) | NO: 8) | NO: 84) | NO: 23) | ID NO: |
| | (SEQ ID | | | | | | | | 48) |
| | NO: 40) | | | | | | | | |
| G3CP | TRVDQSPSS | DANYGLAA | TYWYRKNP | SSNKERIS | GRYSESV | NKGTM | SFTLTISSLQPEDSA | YPWGAGAPYNV | DGAGTKVE |
| G4 | LSASVGDRV | (SEQ IS | G (SEQ ID | IS (SEQ ID | (SEQ ID | (SEQ ID | TYYCRA (SEQ ID | QWY (SEQ ID | IK (SEQ |
| | TITCVLT | NO: 5) | NO: 43) | NO: 7) | NO: 45) | NO: 9) | NO: 46) | NO: 23) | ID NO: |
| | (SEQ ID | | | | | | | | 49) |
| | NO: 41) | | | | | | | | |
| G3CP | ASVTQSPRS | GANYGLAA | TYWYRKNP | SSNQERI | GRYSESV | NKRTM | SFSLRISSLTVEDSA | YPWGAGAPYNV | DGQGTKLE |
| V15 | ASKETGESL | (SEQ ID | G (SEQ ID | SIS (SEQ | (SEQ ID | (SEQ ID | TYYCKA (SEQ ID | QWY (SEQ ID | VK (SEQ |
| | TITCRVT | NO: 1) | NO: 43) | ID NO: 6) | NO: 45) | NO: 8) | NO: 47) | NO: 23) | ID NO: |
| | (SEQ ID | | | | | | | | 85) |
| | NO: 42) | | | | | | | | |
| 1H8 | TRVDQSPSS | DANYGLAA | TYWYRKNP | SSNKERIS | GRYSESV | NKGTM | SFTLTISSLQPEDSA | YPWGAGAPSSV | DGAGTKVE |
| G4 | LSASVGDRV | (SEQ ID | G (SEQ ID | IS (SEQ ID | (SEQ ID | (SEQ ID | TYYCRA (SEQ ID | QWY (SEQ ID | IK (SEQ |
| | TITCVLT | NO: 5) | NO: 43) | NO: 7) | NO: 45) | NO: 9) | NO: 46) | NO: 19) | ID NO: |
| | (SEQ ID | | | | | | | | 49) |
| | NO: 41) | | | | | | | | |
| 1H8 | ASVTQSPRS | GANYGLAA | TYWYRKNP | SSNQERI | GRYSESV | NKRTM | SFSLRISSLTVEDSA | YPWGAGAPSSV | DGQGTKLE |
| V15 | ASKETGESL | (SEQ ID | G (SEQ ID | SIS (SEQ | (SEQ ID | (SEQ ID | TYYCKA (SEQ | QWY (SEQ ID | VK (SEQ |
| | TITCRVT | NO: 1) | NO: 43) | D NO: 6) | NO: 45) | NO: 8) | NO: 47) | NO: 19) | ID NO: |
| | (SEQ ID | | | | | | | | 85) |
| | NO: 42) | | | | | | | | |
| C3CP | TRVDQSPSS | DANYGLAA | TYWYRKNP | SSNKERIS | GRYSESV | NKGTM | SFTLTISSLQPEDSA | YPWGAGAPRQV | DGAGTKVE |
| G4 | LSASVGDRV | (SEQ ID | G (SEQ ID | IS (SEQ ID | (SEQ ID | (SEQ ID | TYYCRA (SEQ ID | QWY (SEQ ID | IK (SEQ |
| | TITCVLT | NO: 5) | NO: 43) | NO: 7) | NO: 45) | NO: 9) | NO: 46) | NO: 14) | ID NO: |
| | (SEQ ID | | | | | | | | 49) |
| | NO: 41) | | | | | | | | |
| C3CP | ASVTQSPRS | GANYGLAA | TYWYRKNP | SSNQERI | GRYSESV | NKRTM | SFSLRISSLTVEDSA | YPWGAGAPRQV | DGQGTKLE |
| V15 | ASKETGESL | (SEQ ID | G (SEQ ID | SIS (SEQ | (SEQ ID | (SEQ ID | TYYCKA (SEQ ID | QWY (SEQ ID | VK (SEQ |
| | TITCRVT | NO: 1) | NO: 43) | ID NO: 6) | NO: 45) | NO: 8) | NO: 47) | NO: 14) | ID NO: |
| | (SEQ ID | | | | | | | | 85) |
| | NO: 42) | | | | | | | | |

The ROR1-specific antigen binding molecule may comprise the CDR and HV sequences of a clone set out in Table 2 below. In preferred embodiments of the first and/or second aspect of the invention, the ROR1-specific antigen binding molecule has the combined sequence of any of the clones set out in Table 2 below.

TABLE 2
P3A1G1 variant sequences

| Clone | | | | | | | | | |
|---|---|---|---|---|---|---|---|---|---|
| Name | FW1 | CDR1 | FW2 | HV2 | FW3a | HV4 | FW3b | CDR3 | FW4 |
| P3A1 G1 | TRVDQSPSS | GTRYGLY | TYWYRKNP | SSDEERISI | GRYSESV | NKGTK | SFTLTISSLQPEDSA | REARHPWLRQW | DGAGTKVE |
| AE3 | LSASVGDRV | S (SEQ | G (SEQ ID | S (SEQ ID | (SEQ ID | (SEQ | TYYCRA (SEQ ID | Y (SEQ ID | IK (SEQ |
| | TITCVLT | ID NO: | NO: 43) | NO: 31) | NO: 45) | ID NO: | NO: 46) | NO: 39) | ID NO: |
| | (SEQ ID | 25) | | | | 35) | | | 49) |
| | NO: 41) | | | | | | | | |
| P3A1 G1 | TRVDQSPSS | GTRYGLY | TYWYRKNP | SSDEERISI | GRYSESV | NKGTK | SFTLTISSLQPEDSA | REARHPWLRQW | DGAGTKVE |
| AE3.S | LSASVGDRV | SS (SEQ | G (SEQ ID | S (SEQ ID | SEQ ID | (SEQ | TYYCRA (SEQ ID | Y (SEQ ID | IK (SEQ |
| | TITCVLT | ID NO: | NO: 43) | NO: 31) | NO: 45) | ID NO: | NO: 46) | NO: 39) | ID NO: |
| | (SEQ ID | 26) | | | | 35) | | | 49) |
| | NO: 41) | | | | | | | | |
| P3A1 G1 | TRVDQSPSS | DTRYALY | TYWYRKNP | STDEERISI | GRYSESV | NKGSK | SFTLTISSLQPEDSA | REARHPWLRQW | DGAGTKVE |
| NAC6 | LSASVGDRV | S (SEQ | G (SEQ ID | G (SEQ ID | (SEQ ID | (SEQ | TYYCRA (SEQ ID | Y (SEQ ID | IK (SEQ |
| | TITCVLT | ID NO: | NO: 43) | NO: 32) | NO: 45) | ID NO: | NO: 46) | NO: 39) | ID NO: |
| | (SEQ ID | 27) | | | | 36) | | | 49) |
| | NO: 41) | | | | | | | | |
| P3A1 G1 | TRVDQSPSS | DTRYALY | TYWYRKNP | STDEERISI | GRYSESV | NKGSK | SFTLTISSLQPEDSA | REARHPWLRQW | DGAGTKVE |
| NAC6.S | LSASVGDRV | SS (SEQ | G (SEQ ID | G (SEQ ID | (SEQ ID | (SEQ | TYYCRA (SEQ ID | Y (SEQ ID | IK (SEQ |
| | TITCVLT | ID NO: | NO: 43) | NO: 32) | NO: 45) | ID NO: | NO: 46) | NO: 39) | ID NO: |
| | (SEQ ID | 28) | | | | 36) | | | 49) |
| | NO: 41) | | | | | | | | |
| P3A1 G1 | TRVDQSPSS | GTKYGLY | TYWYRKNP | SPNKDRMII | GRYSESV | NNGTK | SFTLTISSLQPEDSA | REARHPWLRQW | DGAGTKVE |
| NAG8 | LSASVGDRV | A (SEQ | G (SEQ ID | G (SEQ | (SEQ ID | (SEQ | TYYCRA (SEQ ID | Y (SEQ ID | IK (SEQ |
| | TITCVLT | ID NO: | NO: 43) | ID NO: | NO: 45) | ID NO: | NO: 46) | NO: 39) | ID NO: |
| | (SEQ ID | 29) | | 33) | | 37) | | | 49) |
| | NO: 41) | | | | | | | | |
| P3A1 G1 | TRVDQSPSS | GTKYGLY | TYWYRKNP | SPNKDRMII | GRYSESV | NNGTK | SFTLTISSLQPEDSA | REARHPWLRQW | DGAGTKVE |
| NAG8.S | LSASVGDRV | AS (SEQ | G (SEQ ID | G (SEQ | (SEQ ID | (SEQ | TYYCRA (SEQ ID | Y (SEQ ID | IK (SEQ |
| | TITCVLT | ID NO: | NO: 43) | ID NO: | NO: 45) | ID NO: | NO: 46) | NO: 39) | ID NO: |
| | (SEQ ID | 30) | | 33) | | 37) | | | 49) |
| | NO: 41) | | | | | | | | |
| P3A1 G1 | TRVDQSPSS | GTKYGLY | TYWYRKNP | STDKERIII | GRYSESV | NNRSK | SFTLTISSLQPEDSA | REARHPWLRQW | DGAGTKVE |
| AF7.S | LSASVGDRV | AS (SEQ | G (SEQ ID | G (SEQ ID | (SEQ ID | (SEQ | TYYCRA (SEQ ID | Y (SEQ ID | IK (SEQ |
| | TITCVLT | ID NO: | NO: 43) | NO: 34) | NO: 45) | ID NO: | NO: 46) | NO: 39) | ID NO: |
| | (SEQ ID | 30) | | | | 38) | | | 49) |
| | NO: 41) | | | | | | | | |

All possible combinations and permutations of the framework regions, complementarity determining regions and hypervariable regions listed herein are explicitly contemplated herein.

Sequence identity referenced in relation to the molecules of the invention may be judged at the level of individual CDRs, HVs or FWs, combined CDRs, HVs or FWs, or it may be judged over the length of the entire molecule. The CDR, HV and FW sequences described may also be longer or shorter, whether that be by addition or deletion of amino acids at the N- or C-terminal ends of the sequence or by insertion or deletion of amino acids with a sequence.

Framework region FW1 is preferably from 20 to 28 amino acids in length, more preferably from 22 to 26 amino acids in length, still more preferably from 23 to 25 amino acids in length. In certain preferred embodiments, FW1 is 26 amino acids in length. In other preferred embodiments, FW1 is 25 amino acids in length. In still other preferred embodiments, FW1 is 24 amino acids in length.

In alternative definitions, CDR region CDR1 is preferably from 7 to 11 amino acids in length, more preferably from 8 to 10 amino acids in length. In certain preferred embodiments, CDR1 is 9 amino acids in length. In other preferred embodiments, CDR1 is 8 amino acids in length.

Framework region FW2 is preferably from 6 to 14 amino acids in length, more preferably from 8 to 12 amino acids in length. In certain preferred embodiments, FW2 is 12 amino acids in length. In other preferred embodiments, FW2 is 10 amino acids in length. In other preferred embodiments, FW2 is 9 amino acids in length. In other preferred embodiments, FW2 is 8 amino acids in length.

In alternative definitions, Hypervariable sequence HV2 is preferably from 4 to 11 amino acids in length, more preferably from 5 to 10 amino acids in length. In certain preferred embodiments, HV2 is 10 amino acids in length. In certain preferred embodiments, HV2 is 9 amino acids in length. In other preferred embodiments, HV2 is 6 amino acids in length.

Framework region FW3a is preferably from 6 to 10 amino acids in length, more preferably from 7 to 9 amino acids in length. In certain preferred embodiments, FW3a is 8 amino acids in length. In certain preferred embodiments, FW3a is 7 amino acids in length.

In alternative definitions, Hypervariable sequence HV4 is preferably from 3 to 7 amino acids in length, more preferably from 4 to 6 amino acids in length. In certain preferred embodiments, HV4 is 5 amino acids in length. In other preferred embodiments, HV4 is 4 amino acids in length.

Framework region FW3b is preferably from 17 to 24 amino acids in length, more preferably from 18 to 23 amino acids in length, still more preferably from 19 to 22 amino acids in length. In certain preferred embodiments, FW3b is 21 amino acids in length. In other preferred embodiments, FW3b is 20 amino acids in length.

In alternative definitions, CDR region CDR3 is preferably from 8 to 21 amino acids in length, more preferably from 9 to 20 amino acids in length, still more preferably from 10 to 19 amino acids in length. In certain preferred embodiments, CDR3 is 17 amino acids in length. In other preferred embodiments, CDR3 is 14 amino acids in length. In still other preferred embodiments, CDR3 is 12 amino acids in length. In yet other preferred embodiments, CDR3 is 10 amino acids in length.

Framework region FW4 is preferably from 7 to 14 amino acids in length, more preferably from 8 to 13 amino acids in length, still more preferably from 9 to 12 amino acids in length. In certain preferred embodiments, FW4 is 12 amino acids in length. In other preferred embodiments, FW4 is 11 amino acids in length. In still other preferred embodiments, FW4 is 10 amino acids in length. In yet other preferred embodiments, FW4 is 9 amino acids in length.

In one embodiment of the ROR1-specific antigen binding molecule:
FW1 is a framework region of from 20 to 28 amino acids;
FW2 is a framework region of from 6 to 14 amino acids;
FW3a is a framework region of from 6 to 10 amino acids;
FW3b is a framework region of from 17 to 24 amino acids; and/or
FW4 is a framework region of from 7 to 14 amino acids.

In one embodiment of the ROR1-specific antigen binding molecule:
FW1 has an amino acid sequence selected from the group consisting of: ASVNQTPRTATKETGESLTINCVVT (SEQ ID NO: 40), TRVDQSPSSLSASVGDRVTITCVLT (SEQ ID NO: 41) and ASVTQSPRSASKETGESLTITCRVT (SEQ ID NO: 42), or a functional variant of any thereof with a sequence identity of at least 45%;
FW2 has an amino acid sequence according to TYWYRKNPG (SEQ ID NO: 43), or a functional variant of any thereof with a sequence identity of at least 45%;
FW3a has an amino acid sequence selected from the group consisting of: GRYVESV (SEQ ID NO: 44) and GRYSESV (SEQ ID NO: 45), or a functional variant of any thereof with a sequence identity of at least 45%;
FW3b has an amino acid sequence selected from the group consisting of: SFSLRIKDLTVADSATYYCKA (SEQ ID NO: 84), SFTLTISSLQPEDSATYYCRA (SEQ ID NO: 46) and SFSLRISSLTVEDSATYYCKA (SEQ ID NO: 47), or a functional variant of any thereof with a sequence identity of at least 45%;
and/or
FW4 has an amino acid sequence selected from the group consisting of: DGAGTVLTVN (SEQ ID NO: 48), DGAGTKVEIK (SEQ ID NO: 49) or DGQGTKLEVK (SEQ ID NO: 85) or a functional variant of any thereof with a sequence identity of at least 45%.

The ROR1-specific antigen binding molecule of the present invention may be humanized. The ROR1-specific antigen binding molecule of the present invention may be de-immunized. The B1 loop variants on the humanised backbones G4 and V15 described herein are humanised. As P3A1G1 is already humanised all loop variants of P3A1G1 are humanised. Examples of humanised sequences of the invention include, but are not limited to:
B1G4
G3CP G4
G3CP V15
1H8 G4
1H8 V15
C3CP G4
C3CPV15
P3A1 G1 AE3
P3A1 G1 AE3.S
P3A1 G1 NAC6
P3A1 G1 NAC6.S
P3A1 G1 NAG8
P3A1 G1 NAG8.S
P3A1 G1 AF7.S

It will be appreciated by the skilled person that the humanised ROR1-specific antigen binding molecules described herein may be further humanised, for instance by substituting further FW region amino acids with amino acids of DPK-9.

The ROR1-specific antigen binding molecule of the present invention may also be conjugated to a detectable label, dye, toxin, drug, pro-drug, radionuclide or biologically active molecule.

Preferably, the ROR1-specific antigen binding molecule does not bind to receptor tyrosine kinase-like orphan receptor 2 (ROR2). More preferably, the ROR1-specific antigen binding molecule binds to both human ROR1 and murine ROR1 (mROR1). Yet more preferably, the ROR1-specific antigen binding molecule binds to deglycosylated ROR1.

Certain ROR1-specific antigen binding molecules of the invention may not bind to a linear peptide sequence selected from:

(SEQ ID NO: 91)
YMESLHMQGEIENQI
(SEQ ID NO: 92)
CQPWNSQYPHTHTFTALRFP
(SEQ ID NO: 93)
RSTIYGSRLRIRNLDTTDTGYFQ
(SEQ ID NO: 94)
QCVATNGKEVVSSTGVLFVKFGPPPTASPGYSDEYE

Preferably, the ROR1-specific antigen binding molecule selectively interacts with ROR1 protein with an affinity constant of approximately 0.01 to 50 nM, preferably 0.1 to 30 nM, even more preferably 0.1 to nM. An affinity constant may be measured by Bio-layer interferometry (BLI). For monomers the interaction is 1:1. For the VNAR-hFc format the inventors have used two approaches. One where the ROR1 is immobilized and thus a bi-valent VNAR-hFc binds with an apparent K_{D} as the avidity effect comes into play. The other approach is in a 1:1 format whereby the VNAR-hFc is immobilized and ROR1 is flowed across the surface thus giving the K_{D} for ‘true’ 1:1 binding. Typically, where used herein affinity constants refer to those measured by Bio-layer interferometry (BLI) using the 1:1 binding format. By this method, for example, G3CP and G3CP G4 are within the 0.1-10 nM range. Of the P3A1 G1 loop variants examples have K_{D} values of 5.0 nM (AE3), 13.8 nM (NAC6) and 12.2 nM (NAG8).

Furthermore, the ROR1-specific antigen binding molecule is preferably capable of mediating killing of ROR1-expressing tumour cells or is capable of inhibiting cancer cell proliferation.

The ROR1-specific antigen binding molecule may also be capable of being endocytosed upon binding to ROR1. In other embodiments, the ROR1-specific antigen binding molecule may not be endocytosed upon binding to ROR1.

In a third aspect of the present invention, there it is provided a recombinant fusion protein comprising a specific antigen binding molecule of the first or second aspect. Preferably, in the recombinant fusion protein of the third aspect, the specific antigen binding molecule is fused to one or more biologically active proteins. The specific antigen binding molecule may be fused to one or more biologically active proteins via one or more linker domains. Preferred linkers include but are not limited to [G4S]x, where x is 1, 2, 3, 4, 5, or 6. Particular preferred linkers are [G4S]3 (SEQ ID NO: 86) and [G4S]5 (SEQ ID NO: 87) Other preferred linkers include the sequences PGVQPSP (SEQ ID NO: 88), PGVQPSPGGGGS (SEQ ID NO: 89) and PGVQPAPGGGGS (SEQ ID NO: 90). These linkers may be particularly useful when recombinant fusion proteins are expressed in different expression systems that differ in glycosylation patterns, such as CHO and insect, and those that do not glycosylate expressed proteins (e.g. E. coli). Any recombinant fusion protein sequence disclosed herein comprising a [G4S]3 linker may alternatively possess any other linker sequence disclosed herein.

It will also be appreciated that the fusion proteins of the invention can be constructed in any order, i.e., with the ROR1-specific antigen binding molecule at the N-terminus, C-terminus, or at neither terminus (e.g. in the middle of a longer amino acid sequence).

Preferred biologically active proteins include, but are not limited to an immunoglobulin, an immunoglobulin Fc region, a fragment of an immunoglobulin Fc region, an Fc heavy chain, a CH₂ region, a CH₃ region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein (affibodies, centyrins, darpins etc.). A particularly preferred biologically active protein is an immunoglobulin Fc region. Other preferred fusion proteins include VNAR-VNAR and VNAR-VNAR-VNAR.

In one embodiment, the at least one biologically active protein is an immunoglobulin Fc region.

Therefore, the recombinant fusion protein may comprise a sequence according to SEQ ID NO: 186, SEQ ID NO: 187, SEQ ID NO: 183, SEQ ID NO: 184, or SEQ ID NO: 185.

G3CP-hFc
(SEQ ID NO: 186)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVNGGGGSGGGGSGGGGSEPKSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
G3CPG4-hFc
(SEQ ID NO: 187)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIKGGGGSGGGGSGGGGSEPKSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8-hFc
(SEQ ID NO: 183)
ASVNQTPRTATKETGESLTINCVVTGANYGLAATYWYRKNPGSSNQERIS
ISGRYVESVNKRTMSFSLRIKDLTVADSATYYCKAYPWGAGAPSSVQWYD
GAGTVLTVNGGGGSGGGGSGGGGSEPKSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8 G4-hFc
(SEQ ID NO: 184)
TRVDQSPSSLSASVGDRVTITCVLTDANYGLAATYWYRKNPGSSNKERIS
ISGRYSESVNKGTMSFTLTISSLQPEDSATYYCRAYPWGAGAPSSVQWYD
GAGTKVEIKGGGGSGGGGSGGGGSEPKSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8 V15-hFc
(SEQ ID NO: 185)
ASVTQSPRSASKETGESLTITCRVTGANYGLAATYWYRKNPGSSNQERIS
ISGRYSESVNKRTMSFSLRISSLTVEDSATYYCKAYPWGAGAPSSVQWYD
GQGTKLEVKGGGGSGGGGSGGGGSEPKSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK

In a further embodiment, the at least one biologically active protein is an immunoglobulin Fc region further modified to comprise an S to C mutation.

Therefore, the recombinant fusion protein may comprise a sequence according to SEQ ID NO: 178, SEQ ID NO: 179, SEQ ID NO: 180, SEQ ID NO: 181, or SEQ ID NO: 182.

G3CP-hFc(S239C)
(SEQ ID NO: 178)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVNGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
G3CPG4-hFc(S239C)
(SEQ ID NO: 179)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIKGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8-hFc (S239C)
(SEQ ID NO: 180)
ASVNQTPRTATKETGESLTINCVVTGANYGLAATYWYRKNPGSSNQERIS
ISGRYVESVNKRTMSFSLRIKDLTVADSATYYCKAYPWGAGAPSSVQWYD
GAGTVLTVNGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8 G4-hFc (S239C)
(SEQ ID NO: 181)
TRVDQSPSSLSASVGDRVTITCVLTDANYGLAATYWYRKNPGSSNKERIS
ISGRYSESVNKGTMSFTLTISSLQPEDSATYYCRAYPWGAGAPSSVQWYD
GAGTKVEIKGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
1H8 V15-hFc (S239C)
(SEQ ID NO: 182)
ASVTQSPRSASKETGESLTITCRVTGANYGLAATYWYRKNPGSSNQERIS
ISGRYSESVNKRTMSFSLRISSLTVEDSATYYCKAYPWGAGAPSSVQWYD
GQGTKLEVKGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK

In one embodiment, the at least one biologically active protein is a fragment of an immunoglobulin Fc region selected from the group consisting of an Fc heavy chain, a CH₂ region and a CH₃ region.

In one embodiment, the fragment of an immunoglobulin Fc region is an Fc heavy chain.

In one embodiment, the fragment of an immunoglobulin Fc region is engineered to dimerize with a second fragment of an immunoglobulin Fc region.

As used herein, an immunoglobulin Fc region that is “engineered to dimerise” may comprise at least one amino acid substitution. Typically, the at least one amino acid substitution promotes and/or makes more energetically favourable, an interaction and/or association with a second fragment of an immunoglobulin Fc region, which thus promotes dimerization and/or makes dimerization more energetically favourable. Such recombinant fusion proteins may have particular utility in the preparation of bi-specific and/or bi-paratopic binders.

Methods for generating Fc based bi-specific and/or bi-paratopic binders, through pairing of two distinct Fc heavy chains that are engineered to dimerize, are known in the art. These methods enable an Fc region to be assembled from two different heavy chains, each fused to a target binding domain or sequence with different binding characteristics. The target binding domains or sequences can be directed to different targets to generate multi-specific binders and/or to different regions or epitopes on the same target to generate bi-paratopic binding proteins. Multiple binding domains or sequences can be fused to the Fc sequences to create multi-specific or multi-paratopic binders or both multi-specific multi-paratopic binders within the same protein. Methods to generate these asymmetric bispecific and/or bi-paratopic binders through heterodimerisation of two different Fc heavy chains, or fragments thereof, include but are not limited to: Knobs-into-holes (Y-T), Knobs-into-holes (CW-CSAV), CH₃ charge pair, Fab-arm exchange, SEED technology, BEAT technology, HA-TF, ZW1 approach, Biclonic approach, EW-RVT and Triomab See for example, Brinkman & Kontermann, (2017) mAbs, 9:2, 182-212; Klein et al (2012) mAbs 4:6, 653-663; Wang et al (2019) Antibodies, 8, 43; and Dietrich et al (2020) BBA —Proteins and Proteomics 1868 140250; each of which is incorporated herein by reference in its entirety.

In one embodiment, the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH₃ charge pairing, Fab-arm exchange, SEED technology, BEAT technology, HA-TF, ZW1 approach, Biclonic approach, EW-RVT and Triomab. Features of each of these methods are described in detail in connection with the fourth aspect of the invention and are likewise contemplated as relating to the third aspect of the invention.

In one embodiment, one or more residues of the fragment of the immunoglobulin Fc region comprises one or more amino acid substitution suitable for heterodimerization with a second fragment of an immunoglobulin Fc region comprising one or more corresponding amino acid mutation.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

Any part of the fusion protein of the invention may be engineered to enable conjugation. In a preferred example, where an immunoglobulin Fc region is used, it may be engineered to include a cysteine residue as a conjugation site. Preferred introduced cysteine residues include, but are not limited to S252C and S473C (Kabat numbering), which correspond to S239C and S442C in EU numbering, respectively. In some embodiments, any of the fusion proteins disclosed herein may comprise the S239C point mutation. In some embodiments, any of the fusion proteins disclosed herein may comprise the S442C point mutation. In some embodiments, any of the fusion proteins disclosed herein may comprise both S239C and S442C point mutations. It is explicitly contemplated herein that sequence of any of the fusion proteins disclosed herein may be modified to include an S239C and/or S442C point mutation.

In accordance with the third aspect, recombinant fusions comprising multiple VNAR domains are provided. Accordingly, the recombinant fusions of the invention may be dimers, trimers or higher order multimers of VNARs. In such recombinant fusions, the specificity of each VNAR may be the same or different. Recombinant fusions of the invention include, but are not limited to, bi-specific or tri-specific molecules in which each VNAR domain binds to a different antigen, or to different epitopes on a single antigen (bi-paratopic binders). The term “bi-paratopic” as used herein is intended to encompass molecules that bind to multiple epitopes on a given antigen. Molecules that bind three or more eptiopes on a given antigen are also contemplated herein and where the term “bi-paratopic” is used, it should be understood that the potential for tri-paratopic or multi-paratopic molecules is also encompassed.

Also in accordance with the third aspect, recombinant fusions are provided which include a ROR1-specific antigen binding molecule of the first aspect and a humanised VNAR domain. Humanised VNAR domains may be referred to as soloMERs and include but are not limited to the VNAR BA11, which is a humanised VNAR that binds with high affinity to human serum albumin.

Examples of bi-paratopic and multivalent fusion proteins include, but are not limited to:
B1-G3CP
G3CP-BA11
BA11-G3CP
1H8-BA11
BA11-1H8
G3CP V15-BA11
G3CP G4-BA11
B1-G3CP Cys
G3CP-BA11 Cys
BA11-G3CP Cys
1 H8-BA11 Cys
BA11-1H8 Cys
G3CP V15-BA11 Cys
G3CP G4-BA11 Cys
P3A1G1AE3-(L₂)-G3CPG4
G3CPG4 (L₂)—P3A1G1AE3
P3A1G1AE3-(L₂)-G3CPG4 Cys
G3CPG4-(L₂)-P3A1G1AE3 Cys
P3A1-(L₂)-BA11-(L₂)-G3CP
P3A1-(L₂)-G3CP-(L₂)-BA11
BA11-(L₂)-G3CP-(L₂)-P3A1
BA11-(L₂)-P3A1-(L₂)-G3CP
P3A1-(L₂)-BA11-(L₂)-1H8
BA11-(L₂)-P3A1-(L₂)-1H8
P3A1-(L₂)-BA11-(L₂)-G3CP Cys
P3A1-(L₂)-G3CP-(L₂)-BA11 Cys
BA11-(L₂)-G3CP-(L₂)-P3A1 Cys
BA11-(L₂)-P3A1-(L₂)-G3CP Cys
P3A1-(L₂)-BA11-(L₂)-1H8 Cys
BA11-(L₂)-1P3A1-(L₂)-1H8 Cys
Wherein:

G3CP is
(SEQ ID NO: 50)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVN
1H8 is
(SEQ ID NO: 61)
ASVNQTPRTATKETGESLTINCVVTGANYGLAATYWYRKNPGSSNQERIS
ISGRYVESVNKRTMSFSLRIKDLTVADSATYYCKAYPWGAGAPSSVQWYD
GAGTVLTVN
G3CP G4 is
(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK
G3CP V15 is
(SEQ ID NO: 72)
ASVTQSPRSASKETGESLTITCRVTGANYGLAATYWYRKNPGSSNQERIS
ISGRYSESVNKRTMSFSLRISSLTVEDSATYYCKAYPWGAGAPYNVQWYD
GQGTKLEVK
BA11 is
(SEQ ID NO: 95)
TRVDQSPSSLSASVGDRVTITCVLTDTSYPLYSTYWYRKNPGSSNKEQIS
ISGRYSESVNKGTKSFTLTISSLQPEDSATYYCRAMSTNIWTGDGAGTKV
EIV
P3A1 G1 AE3 is
(SEQ ID NO: 77)
TRVDQSPSSLSASVGDRVTITCVLTGTRYGLYSTYWYRKNPGSSDEERIS
ISGRYSESVNKGTKSFTLTISSLQPEDSATYYCRAREARHPWLRQWYDGA
GTKVEIK

and
where no linker is defined (−) corresponds to the linker Wobbe-G5S, which in turn is PGVQPSPGGGGGS (SEQ ID NO: 96)
(L₂)- corresponds to the linker Wobbe-G4S-GM, which in turn is PGVQPAPGGGGS (SEQ ID NO: 90) Cys—corresponds to a Cys containing C-terminal tag—for example QACKAHHHHHHGAEFEQKLISEEDL (SEQ ID NO: 97)

Recombinant bi-paratopic fusion protein dimers can also be made by fusing any recombinant fusion protein disclosed herein, in particular the loop library variants disclosed herein, onto one arm of an Fc fusion and by fusing binders to a different ROR1 epitope onto the other.

In certain embodiments, the specific binding molecules or recombinant fusions of the invention may be expressed with N- or C-terminal tags to assist with purification. Examples include but are not limited to His₆ and/or Myc. In addition, the N- or C-terminal tag may be further engineered to include additional cysteine residues to serve as conjugation points. It will therefore be appreciated that reference to specific binding molecules or recombinant fusions in all aspects of the invention is also intended to encompass such molecules with a variety of N- or C-terminal tags, which tags may also include additional cysteines for conjugation.

Additional recombinant fusions are listed below. It will be appreciated that not every combination of linker and VNAR or fusion partner is listed below. However, all such combinations are expressly encompassed by the present invention.

Monovalent-BA11 fusions
BA11-G3CP
G3CP-BA11
BA11-G3CPG4
G3CPG4-BA1
P3A1G1 AE3-BA11
BA11-P3A1G1 AE3
Divalent-BA11 fusions
P3A1G1 AE3-P3A1G1 AE3-BA11
BA11-P3A1G1 AE3-P3A1G1 AE3
P3A1G1 AE3-BA11-P3A1G1 AE3
G3CP-G3CP-BA11
G3CP-BA11-G3CP
BA11-G3CP-G3CP
G3CPG4-G3CPG4-BA11
G3CPG4-BA11-G3CPG4
BA11-G3CPG4-G3CPG4
B1G4-B1G4-BA11
B1G4-BA11-B1G4
BA11-B1G4-B1G4
Biparatopic Dimers
G3CP-P3A1G1 AE3
P3A1G1 AE3-G3CP
G3CPG4-P3A1G1 AE3
P3A1G1 AE3-G3CPG4
Dimeric biparatopic BA11 fusions
G3CP-P3A1G1 AE3-BA11
P3A1G1 AE3-G3CP-BA11
G3CP-BA11-P3A1G1 AE3
P3A1G1 AE3-BA11-G3CP
G3CPG4-P3A1G1 AE3-BA11
P3A1G1 AE3-G3CPG4-BA11
G3CPG4-BA11-P3A1G1 AE3
P3A1G1 AE3-BA11-G3CPG4
B1G4-P3A1G1 AE3-BA11
P3A1G1 AE3-B1G4-BA11
B1G4-BA11-P3A1G1 AE3
P3A1G1 AE3-BA11-B1G4

Where the linkers between the VNAR domains are preferentially, but not limited to (G4S)₅ (SEQ ID NO: 87), (G4S)₃ (SEQ ID NO: 86), (G4S)₇ (SEQ ID NO: 116), PGVQPSPGGGGS (SEQ ID NO: 89) (Wobbe-G4S), PGVQPAPGGGGS (SEQ ID NO: 90) (Wobbe-G4S GM), PGVQPCPGGGGGS (SEQ ID NO: 177) (WobbeCys-G4S) and wherein different combinations of different linkers can be combined within the same construct. The WobbeCys-G4S sequence also contains a single cysteine residue to facilitate site-selective bioconjugation of payloads to the proteins, in this linker, using thiol mediated chemical coupling strategies. The use of this linker sequence for bioconjugation is advantageous as reoxidation and capping of the reduced cysteine is minimal, leading to high yielding conversion of the protein to the corresponding conjugate in bioconjugation reactions.

Whereby, additional C-terminal (or N-terminal) tag sequences may or may not be present.

C-terminal tags include, but are not limited to, tags that contain poly-Histidine sequences to facilitate purification (such as His₆), contain c-Myc sequences (such as EQKLISEEDL (SEQ ID NO: 112)) to enable detection and/or contain Cysteine residues to enable labelling and bioconjugation using thiol reactive payloads and probes and combinations thereof. Preferential C-terminal tags include but are not limited to:

(SEQ ID NO: 98)
QASGAHHHHHHGAEFEQKLISEEDL
(SEQ ID NO: 99)
QACGAHHHHHHGAEFEQKLISEEDL
(SEQ ID NO: 97)
QACKAHHHHHHGAEFEQKLISEEDL
(SEQ ID NO: 100)
AAAHHHHHHGAEFEQKLISEEDL
(SEQ ID NO: 101)
ACAHHHHHHGAEFEQKLISEEDL
(SEQ ID NO: 102)
QASGAHHHHHH
(SEQ ID NO: 103)
QACGAHHHHHH
(SEQ ID NO: 104)
QACKAHHHHHH
(SEQ ID NO: 105)
AAAHHHHHH
(SEQ ID NO: 106)
ACAHHHHHH
(SEQ ID NO: 107)
QASGA
(SEQ ID NO: 108)
QACGA
(SEQ ID NO: 109)
QACKA
(SEQ ID NO: 110)
ACA
(SEQ ID NO: 111)
SAPSA
Wherein:
G3CP is
(SEQ ID NO: 50)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVN
G3CP G4 is
(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK
BA11 is
(SEQ ID NO: 95)
TRVDQSPSSLSASVGDRVTITCVLTDTSYPLYSTYWYRKNPGSSNKEQIS
ISGRYSESVNKGTKSFTLTISSLQPEDSATYYCRAMSTNIWTGDGAGTKV
EIK
P3A1 G1 AE3 is
(SEQ ID NO: 77)
TRVDQSPSSLSASVGDRVTITCVLTGTRYGLYSTYWYRKNPGSSDEERIS
ISGRYSESVNKGTKSFTLTISSLQPEDSATYYCRAREARHPWLRQWYDGA
GTKVEIK
G1G4 is
(SEQ ID NO: 51)
TRVDQSPSSLSASVGDRVTITCVLTDANYGLAATYWYRKNPGSSNKERIS
ISGRYSESVNKGTMSFTLTISSLQPEDSATYYCRAYPWGAGAPWLVQWY
DGAGTKVEIK

As stated above, all combinations of VNAR and linker are expressly encompassed herein.

Humanised derivatives of the VNARs are also encompassed herein.

Also in accordance with the third aspect, recombinant fusions are provided which include a ROR1-specific antigen binding molecule of the first aspect and a recombinant toxin. Examples of recombinant toxins include but are not limited to Pseudomonas exotoxin PE38 and diphtheria toxin.

Also in accordance with the third aspect, recombinant fusions are provided which include a ROR1-specific antigen binding molecule of the first aspect and a recombinant CD3 binding protein. Examples of recombinant ROR1 and CD3 binding agents include but are not limited to:

B1G4-[WGM]-CD3
(SEQ ID NO: 117)
TRVDQSPSSLSASVGDRVTITCVLTDANYGLAATYWYRKNPGSSNKERISISGRYSESVNKGTMSFTL
TISSLQPEDSATYYCRAYPWGAGAPWLVQWYDGAGTKVEIKPGVQPAPGGGGSDIKLQQSGAELAR
PGASVKMSCKTSGYTFTRYTMHWVKQRPGQGLEWIGYINPSRGYTNYNQKFKDKATLTTDKSSSTA
YMQLSSLTSEDSAVYYCARYYDDHYCLDYWGQGTTLTVSSGGGGSGGGGSGGGGSDIQLTQSPAI
MSASPGEKVTMTCRASSSVSYMNWYQQKSGTSPKRWIYDTSKVASGVPYRFSGSGSGTSYSLTISS
MEAEDAATYYCQQWSSNPLTFGAGTKLELKSHHHHHH
G3CP-[WGM]-CD3
(SEQ ID NO: 118)
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNPGVQPAPGGGGSDIKLQQSGAELAR
PGASVKMSCKTSGYTFTRYTMHWVKQRPGQGLEWIGYINPSRGYTNYNQKFKDKATLTTDKSSSTA
YMQLSSLTSEDSAVYYCARYYDDHYCLDYWGQGTTLTVSSGGGGSGGGGSGGGGSDIQLTQSPAI
MSASPGEKVTMTCRASSSVSYMNWYQQKSGTSPKRWIYDTSKVASGVPYRFSGSGSGTSYSLTISS
MEAEDAATYYCQQWSSNPLTFGAGTKLELKSHHHHHH
G3CPG4-[WGM]-CD3
(SEQ ID NO: 119)
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKPGVQPAPGGGGSDIKLQQSGAELAR
PGASVKMSCKTSGYTFTRYTMHWVKQRPGQGLEWIGYINPSRGYTNYNQKFKDKATLTTDKSSSTA
YMQLSSLTSEDSAVYYCARYYDDHYCLDYWGQGTTLTVSSGGGGSGGGGSGGGGSDIQLTQSPAI
MSASPGEKVTMTCRASSSVSYMNWYQQKSGTSPKRWIYDTSKVASGVPYRFSGSGSGTSYSLTISS
MEAEDAATYYCQQWSSNPLTFGAGTKLELKSHHHHHH
P3A1G1AE3-[WGM]-CD3
(SEQ ID NO: 120)
TRVDQSPSSLSASVGDRVTITCVLTGTRYGLYSTYWYRKNPGSSDEERISISGRYSESVNKGTKSFTL
TISSLQPEDSATYYCRAREARHPWLRQWYDGAGTKVEIKPGVQPAPGGGGSDIKLQQSGAELARPG
ASVKMSCKTSGYTFTRYTMHWVKQRPGQGLEWIGYINPSRGYTNYNQKFKDKATLTTDKSSSTAYM
QLSSLTSEDSAVYYCARYYDDHYCLDYWGQGTTLTVSSGGGGSGGGGSGGGGSDIQLTQSPAIMSA
SPGEKVTMTCRASSSVSYMNWYQQKSGTSPKRWIYDTSKVASGVPYRFSGSGSGTSYSLTISSMEA
EDAATYYCQQWSSNPLTFGAGTKLELKSHHHHHH
B1G4-[WGM]-BA11-[G4S]-CD3
(SEQ ID NO: 121)
TRVDQSPSSLSASVGDRVTITCVLTDANYGLAATYWYRKNPGSSNKERISISGRYSESVNKGTMSFTL
TISSLQPEDSATYYCRAYPWGAGAPWLVQWYDGAGTKVEIKPGVQPAPGGGGSTRVDQSPSSLSAS
VGDRVTITCVLTDTSYPLYSTYWYRKNPGSSNKEQISISGRYSESVNKGTKSFTLTISSLQPEDSATYY
CRAMSTNIWTGDGAGTKVEIKGGGGSDIKLQQSGAELARPGASVKMSCKTSGYTFTRYTMHWVKQR
PGQGLEWIGYINPSRGYTNYNQKFKDKATLTTDKSSSTAYMQLSSLTSEDSAVYYCARYYDDHYCLD
YWGQGTTLTVSSGGGGSGGGGSGGGGSDIQLTQSPAIMSASPGEKVTMTCRASSSVSYMNWYQQ
KSGTSPKRWIYDTSKVASGVPYRFSGSGSGTSYSLTISSMEAEDAATYYCQQWSSNPLTFGAGTKLE
LKSHHHHHH
G3CP-[WGM]-BA11-[G4S]-CD3
(SEQ ID NO: 122)
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNPGVQPAPGGGGSTRVDQSPSSLSA
SVGDRVTITCVLTDTSYPLYSTYWYRKNPGSSNKEQISISGRYSESVNKGTKSFTLTISSLQPEDSATY
YCRAMSTNIWTGDGAGTKVEIKGGGGSDIKLQQSGAELARPGASVKMSCKTSGYTFTRYTMHWVKQ
RPGQGLEWIGYINPSRGYTNYNQKFKDKATLTTDKSSSTAYMQLSSLTSEDSAVYYCARYYDDHYCL
DYWGQGTTLTVSSGGGGSGGGGSGGGGSDIQLTQSPAIMSASPGEKVTMTCRASSSVSYMNWYQ
QKSGTSPKRWIYDTSKVASGVPYRFSGSGSGTSYSLTISSMEAEDAATYYCQQWSSNPLTFGAGTKL
ELKSHHHHHH
G3CPG4-[WGM]-BA11-[G4S]-CD3
(SEQ ID NO: 123)
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKPGVQPAPGGGGSTRVDQSPSSLSAS
VGDRVTITCVLTDTSYPLYSTYWYRKNPGSSNKEQISISGRYSESVNKGTKSFTLTISSLQPEDSATYY
CRAMSTNIWTGDGAGTKVEIKGGGGSDIKLQQSGAELARPGASVKMSCKTSGYTFTRYTMHWVKQR
PGQGLEWIGYINPSRGYTNYNQKFKDKATLTTDKSSSTAYMQLSSLTSEDSAVYYCARYYDDHYCLD
YWGQGTTLTVSSGGGGSGGGGSGGGGSDIQLTQSPAIMSASPGEKVTMTCRASSSVSYMNWYQQ
KSGTSPKRWIYDTSKVASGVPYRFSGSGSGTSYSLTISSMEAEDAATYYCQQWSSNPLTFGAGTKLE
LKSHHHHHH
P3A1G1AE3-[WGM]-BA11-[G4S]-CD3
(SEQ ID NO: 124)
TRVDQSPSSLSASVGDRVTITCVLTGTRYGLYSTYWYRKNPGSSDEERISISGRYSESVNKGTKSFTL
TISSLQPEDSATYYCRAREARHPWLRQWYDGAGTKVEIKPGVQPAPGGGGSTRVDQSPSSLSASVG
DRVTITCVLTDTSYPLYSTYWYRKNPGSSNKEQISISGRYSESVNKGTKSFTLTISSLQPEDSATYYCR
AMSTNIWTGDGAGTKVEIKGGGGSDIKLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYCLDYW
GQGTTLTVSSGGGGSGGGGSGGGGSDIQLTQSPAIMSASPGEKVTMTCRASSSVSYMNWYQQKSG
TSPKRWIYDTSKVASGVPYRFSGSGSGTSYSLTISSMEAEDAATYYCQQWSSNPLTFGAGTKLELKS
HHHHHH
P3A1-[WGM]-G3CP-[G4S]-CD3
(SEQ ID NO: 125)
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNPGVQPAPGGGGSASVNQTPRTATKET
GESLTINCVVTGANYGLAATYWYRKNPGSSNQERISISGRYVESVNKRTMSFSLRIKDLTVADSATYY
CKAYPWGAGAPYNVQWYDGAGTVLTVNGGGGSDIKLQQSGAELARPGASVKMSCKTSGYTFTRYT
MHWVKQRPGQGLEWIGYINPSRGYTNYNQKFKDKATLTTDKSSSTAYMQLSSLTSEDSAVYYCARY
YDDHYCLDYWGQGTTLTVSSGGGGSGGGGSGGGGSDIQLTQSPAIMSASPGEKVTMTCRASSSVS
YMNWYQQKSGTSPKRWIYDTSKVASGVPYRFSGSGSGTSYSLTISSMEAEDAATYYCQQWSSNPLT
FGAGTKLELKSHHHHHH
P3A1-[WGM]-G3CPG4-[G4S]-CD3
(SEQ ID NO: 126)
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNPGVQPAPGGGGSTRVDQSPSSLSASV
GDRVTITCVLTDANYGLAATYWYRKNPGSSNKERISISGRYSESVNKGTMSFTLTISSLQPEDSATYYC
RAYPWGAGAPYNVQWYDGAGTKVEIKGGGGSDIKLQQSGAELARPGASVKMSCKTSGYTFTRYTM
HWVKQRPGQGLEWIGYINPSRGYTNYNQKFKDKATLTTDKSSSTAYMQLSSLTSEDSAVYYCARYYD
DHYCLDYWGQGTTLTVSSGGGGSGGGGSGGGGSDIQLTQSPAIMSASPGEKVTMTCRASSSVSYM
NWYQQKSGTSPKRWIYDTSKVASGVPYRFSGSGSGTSYSLTISSMEAEDAATYYCQQWSSNPLTFG
AGTKLELKSHHHHHH
P3A1G1AE3-[WGM]-G3CP-[G4S]-CD3
(SEQ ID NO: 127)
TRVDQSPSSLSASVGDRVTITCVLTGTRYGLYSTYWYRKNPGSSDEERISISGRYSESVNKGTKSFTL
TISSLQPEDSATYYCRAREARHPWLRQWYDGAGTKVEIKPGVQPAPGGGGSASVNQTPRTATKETG
ESLTINCVVTGANYGLAATYWYRKNPGSSNQERISISGRYVESVNKRTMSFSLRIKDLTVADSATYYC
KAYPWGAGAPYNVQWYDGAGTVLTVNGGGGSDIKLQQSGAELARPGASVKMSCKTSGYTFTRYTM
HWVKQRPGQGLEWIGYINPSRGYTNYNQKFKDKATLTTDKSSSTAYMQLSSLTSEDSAVYYCARYYD
DHYCLDYWGQGTTLTVSSGGGGSGGGGSGGGGSDIQLTQSPAIMSASPGEKVTMTCRASSSVSYM
NWYQQKSGTSPKRWIYDTSKVASGVPYRFSGSGSGTSYSLTISSMEAEDAATYYCQQWSSNPLTFG
AGTKLELKSHHHHHH
P3A1G1AE3-[WGM]-G3CPG4-[G4S]-CD3
(SEQ ID NO: 128)
TRVDQSPSSLSASVGDRVTITCVLTGTRYGLYSTYWYRKNPGSSDEERISISGRYSESVNKGTKSFTL
TISSLQPEDSATYYCRAREARHPWLRQWYDGAGTKVEIKPGVQPAPGGGGSTRVDQSPSSLSASVG
DRVTITCVLTDANYGLAATYWYRKNPGSSNKERISISGRYSESVNKGTMSFTLTISSLQPEDSATYYCR
AYPWGAGAPYNVQWYDGAGTKVEIKGGGGSDIKLQQSGAELARPGASVKMSCKTSGYTFTRYTMH
WVKQRPGQGLEWIGYINPSRGYTNYNQKFKDKATLTTDKSSSTAYMQLSSLTSEDSAVYYCARYYDD
HYCLDYWGQGTTLTVSSGGGGSGGGGSGGGGSDIQLTQSPAIMSASPGEKVTMTCRASSSVSYMN
WYQQKSGTSPKRWIYDTSKVASGVPYRFSGSGSGTSYSLTISSMEAEDAATYYCQQWSSNPLTFGA
GTKLELKSHHHHHH
P3A1G1AE3-[WGM]-D3-[G4S]-CD3
(SEQ ID NO: 129)
TRVDQSPSSLSASVGDRVTITCVLTGTRYGLYSTYWYRKNPGSSDEERISISGRYSESVNKGTKSFTL
TISSLQPEDSATYYCRAREARHPWLRQWYDGAGTKVEIKPGVQPAPGGGGSASVNQTPRTATKETG
ESLTINCVLTDTSYGLYSTSWFRKNPGTTDWERMSIGGRYVESVNKRAKSFSLRIKDLTVADSATYYC
KAQSGMAISTGSGHGYNWYDGAGTVLTVNGGGGSDIKLQQSGAELARPGASVKMSCKTSGYTFTRY
TMHWVKQRPGQGLEWIGYINPSRGYTNYNQKFKDKATLTTDKSSSTAYMQLSSLTSEDSAVYYCAR
YYDDHYCLDYWGQGTTLTVSSGGGGSGGGGSGGGGSDIQLTQSPAIMSASPGEKVTMTCRASSSV
SYMNWYQQKSGTSPKRWIYDTSKVASGVPYRFSGSGSGTSYSLTISSMEAEDAATYYCQQWSSNPL
TFGAGTKLELKSHHHHHH

Any CD3 binding sequence, and variants thereof, known in the art can be substituted in above. For examples:

UCL OKT3 sequence (WO2019008379)
(SEQ ID NO: 130)
QVQLVQSGAEVKKPGSSVKVSCKASGYTFTRYTMHWVRQAPGQGLEWMGYINPSRGYTNYNQKFK
DRVTITADKSTSTAYMELSSLRSEDTAVYYCARYYDDHYCLDYWGQGTMVTVSSVEGGSGGSGGSG
GSGGVDDIQMTQSPSSLSASVGDRVTITCSASSSVSYMNWYQQKPGKAPKRLIYDTSKLASGVPSRF
SGSGSGTEFTLTISSLQPEDFATYYCQQWSSNPFTFGQGTKVEIK
Harpoon ID20 (WO2016187594)
(SEQ ID NO: 131)
DIKLQQSGAELARPGASVKMSCKTSGYTFTRYTMHWVKQRPGQGLEWIGYINPSRGYTNYNQKFKD
KATLTTDKSSSTAYMQLSSLTSEDSAVYYCARYYDDHYCLDYWGQGTTLTVSSVEGGSGGSGGSGG
SGGVDDIQLTQSPAIMSASPGEKVTMTCRASSSVSYMNWYQQKSGTSPKRWIYDTSKVASGVPYRF
SGSGSGTSYSLTISSMEAEDAATYYCQQWSSNPLTFGAGTKLELK

According to a fourth aspect, the invention provides a recombinant fusion protein comprising an antigen binding molecule comprising an amino acid sequence represented by the formula (I):

FW1-CDR1-FW2-HV2-FW3a-HV4-FW3b-CDR3-FW4 (I)
wherein
   FW1 is a framework region
   CDR1 is a CDR sequence
   FW2 is a framework region
   HV2 is a hypervariable sequence
   FW3a is a framework region
   HV4 is a hypervariable sequence
   FW3b is a framework region
   CDR3 is a CDR sequence
   FW4 is a framework region
or a functional variant thereof,
wherein the antigen binding molecule is fused to a fragment of an immunoglobulin Fc region wherein the fragment of an immunoglobulin Fc region is engineered to dimerize with a second fragment of an immunoglobulin Fc region.

In one embodiment, the fragment of an immunoglobulin Fc region selected from the group consisting of an Fc heavy chain, a CH₂ region and a CH₃ region.

In one embodiment, the fragment of an immunoglobulin Fc region is an Fc heavy chain.

Fc regions may be engineered to reduce FcγR binding. Therefore, the Fc regions disclosed herein may be engineered to reduce FcγR binding.

As used herein, an immunoglobulin Fc region that is “engineered to dimerise” may comprise at least one amino acid substitution. Typically, the at least one amino acid substitution promotes and/or makes more energetically favourable, an interaction and/or association with a second fragment of an immunoglobulin Fc region, which thus promotes dimerization and/or makes dimerization more energetically favourable. Such recombinant fusion proteins may have particular utility in the preparation of bi-specific and/or bi-paratopic binders.

Methods for generating Fc based bi-specific and/or bi-paratopic binders, through pairing of two distinct Fc heavy chains that are engineered to dimerize, are known in the art. These methods enable an Fc region to be assembled from two different heavy chains, each fused to a target binding domain or sequence with different binding characteristics. The target binding domains or sequences can be directed to different targets to generate multi-specific binders and/or to different regions or epitopes on the same target to generate bi-paratopic binding proteins. Multiple binding domains or sequences can be fused to the Fc sequences to create multi-specific or multi-paratopic binders or both multi-specific multi-paratopic binders within the same protein. Methods to generate these asymmetric bispecific and/or bi-paratopic binders through heterodimerisation of two different Fc heavy chains, or fragments thereof, include but are not limited to: Knobs-into-holes (Y-T), Knobs-into-holes (CW-CSAV), CH₃ charge pair, Fab-arm exchange, SEED technology, BEAT technology, HA-TF, ZW1 approach, Biclonic approach, EW-RVT and Triomab See for example, Brinkman & Kontermann, (2017) mAbs, 9:2, 182-212; Klein et al (2012) mAbs 4:6, 653-663; Wang et al (2019) Antibodies, 8, 43; and Dietrich et al (2020) BBA—Proteins and Proteomics 1868 140250; each of which is incorporated herein by reference in its entirety.

In one embodiment, the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH₃ charge pairing, Fab-arm exchange, SEED technology, BEAT technology, HA-TF, ZW1 approach, Biclonic approach, EW-RVT and Triomab.

Knobs-into-holes (Y-T) may comprise a T366Y substitution in a first CH₃ domain and a Y407T substitution in a second CH₃ domain.

Knobs-into-holes (CW-CSAV) may comprise one or more (preferably all) of the following substitutions in a first CH3 domain: S354C, T366W. Knobs-into-holes (CW-CSAV) may comprise one or more (preferably all) of the following substitutions in a second CH3 domain: Y349C, T366S, L368A, Y407V. Knobs-into-holes (CW-CSAV) may comprise a disulphide bond in CH3.

CH3 charge pairing, may comprise one or more (preferably all) of the following substitutions in a first CH3 domain: K392D, K409D. CH3 charge pairing may comprise one or more (preferably all) of the following substitutions in a second CH3 domain: E356K, D399K.

Fab-arm exchange, may comprise a K409R substitution in a first CH3 domain and a F405L substitution in a second CH3 domain. Fab arm exchange and DuoBody capture the same Fc changes. DuoBody technology, may therefore comprise a K409R substitution in a first CH3 domain and a F405L substitution in a second CH3 domain.

SEED technology may incorporate known substitutions and/or result in an IgG/A chimera. Complementarity in the CH3 interface allowing for a heterodimeric assembly of Fc chains was developed by designing strand-exchange engineered domain (SEED) heterodimers. These SEED CH3 domains are composed of alternating segments derived from human IgA and IgG CH3 sequences (AG SEED CH3 and GA SEED CH3) and were used to generate so-called SEEDbodies, Davis et al (2010) PEDS 23, 4, 195-202 hereby incorporated by reference in its entirety Because molecular models suggested that interaction with FcRn is impaired in the AG SEED CH3, residues at the CH2-CH3 junction were returned to IgG sequences. Pharmacokinetic studies confirmed that the half-life of SEEDbodies was comparable to other Fc fusion proteins and IgG1.

BEAT technology engineers the constant α and β domains of the human T cell receptor into the IgG1 CH3 dimer interface to drive heterodimerisation (Skegro et al (2017) JBC 292(23) 9745-9759). An additional D410Q mutation can further increase heterodimer formation in this system (Stutz & Blein 2020 JBC 295(28) 9392-9408).

HA-TF, may comprise one or more (preferably all) of the following substitutions in a first CH3 domain: S364H, F405A. HA-TF may comprise one or more (preferably all) of the following substitutions in a second CH3 domain: Y349T, T394F.

ZW1 approach, may comprise one or more (preferably all) of the following substitutions in a first CH3 domain: T350V, L351Y, F405A, Y407V. ZW1 approach, may comprise one or more (preferably all) of the following substitutions in a second CH3 domain: T350V, T366L, K392L, T394W.

Biclonic approach, may comprise one or more (preferably all) of the following substitutions in a first CH3 domain: 366K (+351 K). Biclonic approach, may comprise one or more (preferably all) of the following substitutions in a second CH3 domain: 351D or E or D at 349, 368, 349, or 349+355.

EW-RVT, may comprise one or more (preferably all) of the following substitutions in a first CH3 domain: K360E, K409W. EW-RVT, may comprise one or more (preferably all) of the following substitutions in a second CH3 domain: Q347R, D399V, F405T. EW-RVT may comprise a disulphide bond in CH3. A disulphide bridge may be supported by the further incorporation of Y349C to a first CH3 domain and S354C to a second CH3 domain.

Triomabs may be formed by fusing a mouse hybridoma with a rat hybridoma, resulting in production of a bispecific, assymmetric hybrid IgG molecule. Preferential pairing of light chains with its corresponding heavy chain may then occur.

In one embodiment, one or more residues of the fragment of the immunoglobulin Fc region comprises one or more amino acid substitution suitable for knobs-in-holes (KIH) dimerization with a second fragment of an immunoglobulin Fc region comprising one or more corresponding amino acid mutation.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

In one embodiment, the antigen binding molecule is a ROR1 specific antigen binding molecule.

The recombinant fusion protein may comprise a sequence according to SEQ ID NO: 146 or SEQ ID NO: 147.

G3CP hFc (S239C + Y407T)
SEQ ID NO: 146
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNGGGGGGGGSGGGGSEPKSSDKT
HTCPPCPAPELLGGPCVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLTSKLTVDKSRWQQGNVFSC
SVMHEALHNHYTQKSLSLSPGK
G3CPG4 hFc (S239C + Y407T)
SEQ ID NO: 147
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKGGGGSGGGGSGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK

The recombinant fusion protein may comprise a sequence according to SEQ ID NO: 194, SEQ ID NO: 195 or SEQ ID NO 196:

1H8 hFc (S239C + Y407T)
SEQ ID NO: 194
ASVNQTPRTATKETGESLTINCVVTGANYGLAATYWYRKNPGSSNQERISISGRYVESVNKRTMSFSL
RIKDLTVADSATYYCKAYPWGAGAPSSVQWYDGAGTVLTVNGGGGSGGGGSGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK
1H8 G4 hFc (S239C + Y407T)
SEQ ID NO: 195
TRVDQSPSSLSASVGDRVTITCVLTDANYGLAATYWYRKNPGSSNKERISISGRYSESVNKGTMSFTL
TISSLQPEDSATYYCRAYPWGAGAPSSVQWYDGAGTKVEIKGGGGSGGGGSGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK
1H8 v15 hFc (S239C + Y407T)
SEQ ID NO: 196
ASVTQSPRSASKETGESLTITCRVTGANYGLAATYWYRKNPGSSNQERISISGRYSESVNKRTMSFSL
RISSLTVEDSATYYCKAYPWGAGAPSSVQWYDGQGTKLEVKGGGGSGGGGSGGGGSEPKSSDKT
HTCPPCPAPELLGGPCVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLTSKLTVDKSRWQQGNVFSC
SVMHEALHNHYTQKSLSLSPGK

The recombinant fusion protein may comprise a sequence according to SEQ ID NO: 148.

P3A1 hFc (S239C + T366Y)
SEQ ID NO: 148
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNGGGGSGGGGSGGGGSEPKSSDKTHT
CPPCPAPELLGGPCVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTK
NQVSLYCLVKGFYPSDIAVEWESNGQPENNYKTTPPVLDSDGSFFLYSKLTVDKSRWQQGNVFSCS
VMHEALHNHYTQKSLSLSPGK

The recombinant fusion protein may comprise a sequence according to SEQ ID NO: 191 or SEQ ID NO: 192:

G3CP hFc (S239C + T366Y)
SEQ ID NO: 191
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNGGGGSGGGGSGGGGSEPKSSDKT
HTCPPCPAPELLGGPCVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLYCLVKGFYPSDIAVEWESNGQPENNYKTTPPVLDSDGSFFLYSKLTVDKSRWQQGNVFS
CSVMHEALHNHYTQKSLSLSPGK
G3CPG4 hFc (S239C + T366Y)
SEQ ID NO: 192
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKGGGGSGGGGSGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK

The recombinant fusion protein may comprise a sequence according to SEQ ID NO: 197, SEQ ID NO: 198, or SEQ ID NO: 199

1H8 hFc (S239C + T366Y)
SEQ ID NO: 197
ASVNQTPRTATKETGESLTINCVVTGANYGLAATYWYRKNPGSSNQERISISGRYVESVNKRTMSFSL
RIKDLTVADSATYYCKAYPWGAGAPSSVQWYDGAGTVLTVNGGGGSGGGGGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK
1H8 G4 hFc (S239C + T366Y)
SEQ ID NO: 198
TRVDQSPSSLSASVGDRVTITCVLTDANYGLAATYWYRKNPGSSNKERISISGRYSESVNKGTMSFTL
TISSLQPEDSATYYCRAYPWGAGAPSSVQWYDGAGTKVEIKGGGGSGGGGSGGGGSEPKSSDKTH
TCPPCPAPELLGGPCVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK
1H8 v15 hFc (S239C + T366Y)
SEQ ID NO: 199
ASVTQSPRSASKETGESLTITCRVTGANYGLAATYWYRKNPGSSNQERISISGRYSESVNKRTMSFSL
RISSLTVEDSATYYCKAYPWGAGAPSSVQWYDGQGTKLEVKGGGGSGGGGSGGGGSEPKSSDKT
HTCPPCPAPELLGGPCVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLYCLVKGFYPSDIAVEWESNGQPENNYKTTPPVLDSDGSFFLYSKLTVDKSRWQQGNVFS
CSVMHEALHNHYTQKSLSLSPGK

The recombinant fusion protein may comprise a sequence according to SEQ ID NO: 193:

P3A1 hFc (S239C + Y407T) SEQ ID NO: 93:
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNGGGGSGGGGSGGGGSEPKSSDKTHT
CPPCPAPELLGGPCVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLTSKLTVDKSRWQQGNVFSCSV
MHEALHNHYTQKSLSLSPGK

The recombinant fusion protein may be a bi-paratopic dimer comprising any one or any two of SEQ ID NOs 146, 147, 194, 195, 196, 148, 191, 191, 192, 193, 197, 198 and 199. The bi-paratopic dimer may comprise one of SEQ ID NOs 146, 147, 194, 195, 196 and 193 comprising the Y407T point mutation. The bi-paratopic dimer may comprise one of SEQ ID NOs 148, 191, 192, 197, 198 and 199 comprising the T366Y point mutation. The bi-paratopic dimer may comprise SEQ ID NO: 146 and SEQ ID NO: 148 or SEQ ID NO: 147 and SEQ ID NO: 148. Any of the recombinant fusion proteins disclosed herein may be associated with any of the linkers and payloads disclosed herein, Any of the bi-paratopic dimers disclosed herein may be associated with any of the linkers and payloads disclosed herein, Conjugation may be by any one or more S239C residue in the bi-paratopic dimer. Preferably, the bi-paratopic dimer may be associated with the linker and payload vc-PAB-EDA-PNU. Preferably, the bi-paratopic dimer comprises G3CP hFc(S239C+Y407T) (SEQ ID NO: 146) and P3A1 hFc(S239C+T366Y) (SEQ ID NO: 148), conjugated to vc-PAB-EDA-PNU, or G3CPG4 hFc(S239C+Y407T) (SEQ ID NO: 147) and P3A1 hFc(S239C+T366Y) (SEQ ID NO: 148), conjugated to vc-PAB-EDA-PNU which have been shown to be highly efficacious in vivo.

SEQ ID Nos: 146, 147, 194, 195, 196, 148, 191, 191, 192, 193, 197, 198 and 199 include an S239C mutation, for use in conjugation reactions. Where the recombinant fusion protein is not conjugated (for example to an anthracycline (PNU) derivative) the S239C mutation is not needed and position 239 may be an S rather than a C. Accordingly, in alternative embodiments the recombinant fusion protein or bi-paratopic dimer may comprise a sequence according to any one of SEQ ID Nos: 146, 147, 194, 195, 196, 148, 191, 191, 192, 193, 197, 198 and 199 except that each sequence does not include an S239C mutation.

Therefore, the recombinant fusion protein may comprise a sequence according to SEQ ID NO: 165 or SEQ ID NO: 166.

G3CP-hFc (Y407T) SEQ ID NO: 165:
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNGGGGSGGGGSGGGGSEPKSSDKT
HTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLTSKLTVDKSRWQQGNVFSC
SVMHEALHNHYTQKSLSLSPGK
G3CP G4-hFc (Y407T) SEQ ID NO: 166:
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK

The recombinant fusion protein may comprise a sequence according to SEQ ID NO: 200, SEQ ID NO: 201 or SEQ ID NO 202.

1H8 hFc (Y407T)
SEQ ID NO: 200
ASVNQTPRTATKETGESLTINCVVTGANYGLAATYWYRKNPGSSNQERISISGRYVESVNKRTMSFSL
RIKDLTVADSATYYCKAYPWGAGAPSSVQWYDGAGTVLTVNGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK
1H8 G4 hFc (Y407T)
SEQ ID NO: 201
TRVDQSPSSLSASVGDRVTITCVLTDANYGLAATYWYRKNPGSSNKERISISGRYSESVNKGTMSFTL
TISSLQPEDSATYYCRAYPWGAGAPSSVQWYDGAGTKVEIKGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLTSKLTVDKSRWQQGNVFSCS
VMHEALHNHYTQKSLSLSPGK
1H8 v15 hFc (Y407T)
SEQ ID NO: 202
ASVTQSPRSASKETGESLTITCRVTGANYGLAATYWYRKNPGSSNQERISISGRYSESVNKRTMSFSL
RISSLTVEDSATYYCKAYPWGAGAPSSVQWYDGQGTKLEVKGGGGSGGGGSGGGGSEPKSSDKT
HTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLTSKLTVDKSRWQQGNVFSC
SVMHEALHNHYTQKSLSLSPGK

The recombinant fusion protein may comprise a sequence according to SEQ ID NO: 167:

P3A1 hFc (T366Y) SEQ ID NO: 167:
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNGGGGSGGGGSGGGGSEPKSSDKTHT
CPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTK
NQVSLYCLVKGFYPSDIAVEWESNGQPENNYKTTPPVLDSDGSFFLYSKLTVDKSRWQQGNVFSCS
VMHEALHNHYTQKSLSLSPGK

The recombinant fusion protein may comprise a sequence according to SEQ ID NO: 188 or SEQ ID NO: 189:

G3CP hFc (T366Y)
SEQ ID NO: 188
ASVNQTPRTATKETGESLTINCVVTGANYGLAATYWYRKNPGSSNQERISISGRYVESVNKRTMSFSL
RIKDLTVADSATYYCKAYPWGAGAPYNVQWYDGAGTVLTVNGGGGSGGGGSGGGGSEPKSSDKT
HTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLYCLVKGFYPSDIAVEWESNGQPENNYKTTPPVLDSDGSFFLYSKLTVDKSRWQQGNVFS
CSVMHEALHNHYTQKSLSLSPGK
G3CPG4 hFc (T366Y)
SEQ ID NO: 189
TRVDQSPSSLSASVGDRVTITCVLTDANYGLAATYWYRKNPGSSNKERISISGRYSESVNKGTMSFTL
TISSLQPEDSATYYCRAYPWGAGAPYNVQWYDGAGTKVEIKGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK

The recombinant fusion protein may comprise a sequence according to SEQ ID NO: 203, SEQ ID NO: 204, or SEQ ID NO: 205:

1H8 hFc (T366Y)
SEQ ID NO: 203
ASVNQTPRTATKETGESLTINCVVTGANYGLAATYWYRKNPGSSNQERISISGRYVESVNKRTMSFSL
RIKDLTVADSATYYCKAYPWGAGAPSSVQWYDGAGTVLTVNGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK
1H8 G4 hFc (T366Y)
SEQ ID NO: 204
TRVDQSPSSLSASVGDRVTITCVLTDANYGLAATYWYRKNPGSSNKERISISGRYSESVNKGTMSFTL
TISSLQPEDSATYYCRAYPWGAGAPSSVQWYDGAGTKVEIKGGGGSGGGGSGGGGSEPKSSDKTH
TCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELT
KNQVSLYCLVKGFYPSDIAVEWESNGQPENNYKTTPPVLDSDGSFFLYSKLTVDKSRWQQGNVFSC
SVMHEALHNHYTQKSLSLSPGK
1H8 v15 hFc (T366Y)
SEQ ID NO: 205
ASVTQSPRSASKETGESLTITCRVTGANYGLAATYWYRKNPGSSNQERISISGRYSESVNKRTMSFSL
RISSLTVEDSATYYCKAYPWGAGAPSSVQWYDGQGTKLEVKGGGGSGGGGSGGGGSEPKSSDKT
HTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDEL
TKNQVSLYCLVKGFYPSDIAVEWESNGQPENNYKTTPPVLDSDGSFFLYSKLTVDKSRWQQGNVFS
CSVMHEALHNHYTQKSLSLSPGK

The recombinant fusion protein may comprise a sequence according to SEQ ID NO: 190:

P3A1 hFc (Y407T)
SEQ ID NO: 190
TRVDQTPRTATKETGESLTINCVLTDTSYGLYSTSWFRKNPGTTDWERMSIGGRYVESVNKGAKSFS
LRIKDLTVADSATYYCKAREARHPWLRQWYDGAGTVLTVNGGGGSGGGGSGGGGSEPKSSDKTHT
CPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLTSKLTVDKSRWQQGNVFSCSV
MHEALHNHYTQKSLSLSPGK

According to a fifth aspect, the invention provides a recombinant fusion protein dimer comprising
(a) a first recombinant fusion protein, wherein the first recombinant fusion protein is a recombinant fusion protein according to the third or fourth aspects, and
(b) a second recombinant fusion protein, wherein the second recombinant fusion protein comprises a second antigen binding molecule fused to a second fragment of an immunoglobulin Fc region engineered to dimerize with the first fragment of an immunoglobulin Fc region.

In one embodiment, the second fragment of an immunoglobulin Fc region selected from the group consisting of an Fc heavy chain, a CH2 region and a CH3 region.

In one embodiment, the second fragment of an immunoglobulin Fc region is an Fc heavy chain.

In one embodiment, the second fragment of an immunoglobulin Fc region is engineered to dimerize with the second fragment of an immunoglobulin Fc region by a method selected from the group consisting of knobs-into-holes (Y-T), knobs-into-holes (CW-CSAV), CH3 charge pairing, Fab-arm exchange, SEED technology, BEAT technology, HA-TF, ZW1 approach, Biclonic approach, EW-RVT and Triomab.

In one embodiment, one or more residues of the fragment of the immunoglobulin Fc region comprises one or more amino acid substitution suitable for knobs-in-holes (KIH) dimerization with a second fragment of an immunoglobulin Fc region comprising one or more corresponding amino acid mutation.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

Any sequence of a recombinant fusion protein disclosed herein may comprise any one or more amino acid substitution selected from the group consisting of T366Y, Y407T, S354C, T366W, Y349C, T366S, L368A and Y407V. SEQ ID NO: 145 (human Fc region) may therefore be modified by the incorporation of any one or more amino acid substitution selected from the group consisting of T366Y, Y407T, S354C, T366W, Y349C, T366S, L368A and Y407V and incorporated into a recombinant fusion protein as described herein in place of the human Fc region sequence.

In one embodiment, the second antigen binding molecule is a ROR1 specific antigen binding molecule.

In one embodiment, the second specific antigen binding molecule is an immunoglobin, an immunoglobin Fab region, a Fab′, a Fv, a Fv-Fc, a single chain Fv (scFv), scFv-Fc, (scFv)₂, a diabody, a triabody, a tetrabody, a bispecific t-cell engager (BiTE), an intein, a VNAR domain, a single domain antibody (sdAb) or a VH domain.

In one embodiment:
(a) the first recombinant fusion protein comprises a sequence according to SEQ ID NO: 146 or SEQ ID NO: 147, and
(b) the second recombinant fusion protein comprises a sequence according to SEQ ID NO: 148.

According to a sixth aspect, the invention provides a ROR1-specific chimeric antigen receptor (CAR), comprising at least one ROR1-specific antigen binding molecule as defined by the first or second aspects of the invention, fused or conjugated to at least one transmembrane region and at least one intracellular domain.

The present invention also provides a cell comprising a chimeric antigen receptor according to the sixth aspect, which cell is preferably an engineered T-cell.

In a seventh aspect of the invention, there is provided a nucleic acid sequence comprising a polynucleotide sequence that encodes a specific antigen binding molecule, recombinant fusion protein, recombinant fusion protein dimer or chimeric antigen receptor according to the first, second, third, fourth, fifth, or sixth aspects of the invention.

There is also provided a vector comprising a nucleic acid sequence in accordance with the seventh aspect and a host cell comprising such a nucleic acid.

A method for preparing a specific antigen binding molecule, recombinant fusion protein, recombinant fusion protein dimer or chimeric antigen receptor, of the first, second, third, fourth, fifth, or sixth aspect is provided, the method comprising cultivating or maintaining a host cell comprising the polynucleotide or vector described above under conditions such that said host cell produces the specific antigen binding molecule, recombinant fusion protein or chimeric antigen receptor, optionally further comprising isolating the specific antigen binding molecule, recombinant fusion protein or chimeric antigen receptor.

In an eighth aspect of the invention, there is provided a pharmaceutical composition comprising the specific antigen binding molecule, recombinant fusion protein, recombinant fusion protein dimer or chimeric antigen receptor of the first, second, third, fourth, fifth, or sixth aspects. The pharmaceutical composition may contain a variety of pharmaceutically acceptable carriers. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The specific antigen binding molecule, recombinant fusion protein or chimeric antigen receptor of the first, second, third, fourth, fifth, or sixth aspects may be for use in therapy. More specifically, the specific antigen binding molecule, recombinant fusion protein, recombinant fusion protein dimer or chimeric antigen receptor of the first, second, third, fourth, fifth, or sixth aspects may be for use in the treatment of cancer. Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is the use of a specific antigen binding molecule, recombinant fusion protein, recombinant fusion protein dimer or chimeric antigen receptor of the first, second, third, fourth, fifth, or sixth aspects in the manufacture of a medicament for the treatment of a disease in a patient in need thereof.

Furthermore, in accordance with the present invention there is provided a method of treatment of a disease in a patient in need of treatment comprising administration to said patient of a therapeutically effective dosage of a specific antigen binding molecule, recombinant fusion protein, recombinant fusion protein dimer or chimeric antigen receptor of the first, second, third, fourth, fifth, or sixth aspects or a pharmaceutical composition of the sixth aspect.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is a method of assaying for the presence of a target analyte in a sample, comprising the addition of a detectably labelled specific antigen binding molecule of the first or second aspect, or a recombinant fusion protein or recombinant fusion protein of the third or fourth aspect, or a recombinant fusion protein dimer of the fifth aspect, to the sample and detecting the binding of the molecule to the target analyte.

In addition, there is provided herein a method of imaging a site of disease in a subject, comprising administration of a detectably labelled specific antigen binding molecule of the first or second aspect or a detectably labelled recombinant fusion protein or recombinant fusion protein of the third or fourth aspect, or a recombinant fusion protein dimer of the fifth aspect to a subject.

There is also provided herein a method of diagnosis of a disease or medical condition in a subject comprising administration of a specific antigen binding molecule of the first aspect or second aspect, or a recombinant fusion protein or recombinant fusion protein of the third or fourth aspect, or a recombinant fusion protein dimer of the fifth aspect.

Also contemplated herein is an antibody, antibody fragment or antigen-binding molecule that competes for binding to ROR1 with the ROR1-specific antigen binding molecule of the first or second aspect. The term “compete” when used in the context of antigen binding proteins (e.g., neutralizing antigen binding proteins or neutralizing antibodies) means competition between antigen binding proteins as determined by an assay in which the antigen binding protein (e.g., antibody or functional fragment thereof) under test prevents or inhibits specific binding of a the antigen binding molecule defined herein (e.g., specific antigen binding molecule of the first aspect) to a common antigen (e.g., ROR1 in the case of the specific antigen binding molecule of the first or second aspect).

Also described herein is a kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises a ROR1-specific antigen binding molecule of the first or second aspect, a recombinant fusion protein or recombinant fusion protein dimer of the third, fourth or fifth aspect, a chimeric antigen receptor of the sixth aspect or a nucleic acid sequence of the seventh aspect, each being optionally derivatized, wherein presence of antigen in the sample suggests that the subject suffers from cancer. Preferably the antigen comprises ROR1 protein, more preferably an extracellular domain thereof. More preferably, the kit is used to identify the presence or absence of ROR1-positive cells in the sample, or determine the concentration thereof in the sample. The kit may also comprise a positive control and/or a negative control against which the assay is compared and/or a label which may be detected.

The present invention also provides a method for diagnosing a subject suffering from cancer, or a pre-disposition thereto, or for providing a prognosis of the subject's condition, the method comprising detecting the concentration of antigen present in a sample obtained from a subject, wherein the detection is achieved using a ROR1-specific antigen binding molecule of the first or second aspect, a recombinant fusion protein or recombinant fusion protein dimer of the third, fourth or fifth aspect, a chimeric antigen receptor of the sixth aspect or a nucleic acid sequence of the seventh aspect, each being optionally derivatized, and wherein presence of antigen in the sample suggests that the subject suffers from cancer.

Also contemplated herein is a method of killing or inhibiting the growth of a cell expressing ROR1 in vitro or in a patient, which method comprises administering to the cell a pharmaceutically effective amount or dose of (i) ROR1-specific antigen binding molecule of the first or second aspect, a recombinant fusion protein or recombinant fusion protein dimer of the third, fourth or fifth aspect, a nucleic acid sequence of the sixth aspect, or the CAR or cell according the seventh aspect, or (ii) of a pharmaceutical composition of the eighth aspect. Preferably, the cell expressing ROR1 is a cancer cell. More preferably, the ROR1 is human ROR1.

According to a ninth aspect, the invention provides a specific antigen binding molecule comprising an amino acid sequence represented by the formula (II):

X-FW1-CDR1-FW2-HV2-FW3a-HV4-FW3b-CDR3-FW4-Y (II)
wherein
   FW1-CDR1-FW2-HV2-FW3a-HV4-FW3b-CDR3-FW4 is a ROR1-specific antigen binding molecule according to the first or second aspect
   X and Y are optional amino acid sequences
wherein the specific antigen binding molecule is conjugated to a second moiety.

In certain preferred embodiments, the specific antigen binding molecule according to this aspect of the invention may additionally be conjugated to a third, fourth or fifth moiety. Conjugation of further moieties is also contemplated. In some cases, a third, fourth or fifth moiety may be conjugated to the second moiety. Accordingly, it will be understood that any of the moieties according to this aspect of the invention may have additional moieties conjugated thereto. Description of preferred features of the second moiety as set out below apply to the third, fourth, fifth or higher order moiety mutatis mutandis.

Preferably X or Y are individually either absent or selected from the group comprising an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

Preferably, the conjugation is via a cysteine residue in the amino acid sequence of the specific antigen binding molecule. The cysteine residue may be anywhere in the sequence, including in optional sequences X or Y (if present).

The conjugation may be via a thiol, aminoxy or hydrazinyl moiety incorporated at the N-terminus or C-terminus of the amino acid sequence of the specific antigen binding molecule.

Preferably, the second moiety is selected from the group comprising detectable label, dye, toxin, drug, pro-drug, radionuclide or biologically active molecule.

More preferably, the second moiety is at least one toxin selected from the group comprising:
maytansinoids,
auristatins,
anthracyclines, preferably PNU-derived anthracyclines
calicheamicins,
amanitin derivatives, preferably α-amanitin derivatives
tubulysins
duocarmycins
radioisotopes for example alpha-emitting radionuclide, such as 227 Th or 225 Ac
liposomes comprising a toxic payload,
protein toxins
taxanes,
pyrrolbenzodiazepines
indolinobenzodiazepine pseudodimers and/or
spliceosome inhibitors
CDK11 inhibitors
Pyridinobenzodiazepines
Irinotecan and its derivatives

In other preferred embodiments in accordance with this aspect, the second moiety may be from the group comprising an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

In particularly preferred embodiments, the second moiety is a VNAR domain, which may be the same or different to the specific antigen binding molecule according to this aspect. Accordingly, dimers, trimers or higher order multimers of VNAR domains linked by chemical conjugation are explicitly contemplated herein. In such embodiments, each individual VNAR domain may have the same antigen specificity as the other VNAR domains, or they may be different.

In accordance with this aspect, the specific antigen binding molecule may comprise, for example, bi-paratopic specific antigen binding molecules as described in relation to the first to fifth aspects fused to further biologically active molecules (including but not limited to molecules for half-life extension, for example BA11) and then further conjugated to a second moiety, including but not limited to cytotoxic payloads

In accordance with this aspect, the specific antigen binding molecule may be a receptor tyrosine kinase-like orphan receptor 1 (ROR1) specific antigen binding molecule. This may be a ROR1-specific antigen binding molecule of the first or second aspect of the invention. Accordingly, any of the preferred features described in relation to the first, second and third aspects apply mutatis mutandis to the sixth aspect.

The specific antigen binding molecule of the ninth aspect may be for use in therapy. More specifically, the specific antigen binding molecule of the ninth aspect may be for use in the treatment of cancer. Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is the use of a specific antigen binding molecule of the ninthaspect in the manufacture of a medicament for the treatment of a disease in a patient in need thereof.

Pharmaceutical compositions comprising the specific antigen binding molecule of the ninth aspect are also provided. The pharmaceutical composition may contain a variety of pharmaceutically acceptable carriers

Furthermore, in accordance with the present invention there is provided a method of treatment of a disease in a patient in need of treatment comprising administration to said patient of a therapeutically effective dosage of a specific antigen binding molecule of the ninth aspect or a pharmaceutical composition comprising a specific antigen binding molecule of the ninth aspect.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

Also provided herein is a method of assaying for the presence of a target analyte in a sample, comprising the addition of a detectably labelled specific antigen binding molecule of the ninth aspect to the sample and detecting the binding of the molecule to the target analyte.

In addition, there is provided herein a method of imaging a site of disease in a subject, comprising administration of a detectably labelled specific antigen binding molecule of the ninth aspect to a subject.

There is also provided herein a method of diagnosis of a disease or medical condition in a subject comprising administration of a specific antigen binding molecule of the ninth aspect.

Furthermore, any of the features described in respect of any of the above-mentioned aspects of the invention may be combined mutatis mutandis with the other aspects of the invention.

In addition to the sequences mentioned the following sequences are expressly disclosed. Certain of these sequences relate to examples of molecules of the invention described herein:

TABLE 3
additional sequences

| SEQ ID | | |
|---|---|---|
| NO: | Sequence name | Sequence |
| 115 | B1V15 | ASVTQSPRSASKETGESLTITCRVTGANYGLAATYWYRKNPGSSNQERISISGRYSESVNKR |
| | | TMSFSLRISSLTVEDSATYYCKAYPWGAGAPWLVQWYDGQGTKLEVK |
| 151 | human ROR1-His | QETELSVSAELVPTSSWNISSELNKDSYLTLDEPMNNITTSLGQTAELHCKVSGNPPPTI |
| | (CHO) | RWFKNDAPVVQEPRRLSFRSTIYGSRLRIRNLDTTDTGYFQCVATNGKEVVSSTGVLFVK |
| | | FGPPPTASPGYSDEYEEDGFCQPYRGIACARFIGNRTVYMESLHMQGEIENQITAAFTMI |
| | | GTSSHLSDKCSQFAIPSLCHYAFPYCDETSSVPKPRDLCRDECEILENVLCQTEYIFARS |
| | | NPMILMRLKLPNCEDLPQPESPEAANCIRIGIPMADPINKNHKCYNSTGVDYRGTVSVTK |
| | | SGRQCQPWNSQYPHTHTFTALRFPELNGGHSYCRNPGNQKEAPWCFTLDENFKSDLCDIP |
| | | ACDSKDSKEKNKMEHHHHHH |
| 152 | human ROR1 (Ig | SELNKDSYLTLDEPMNNITTSLGQTAELHCKVSGNPPPTIRWFKNDAPVVQEPRRLSFRS |
| | domain) | TIYGSRLRIRNLDTTDTGYFQCVATNGKEVVSSTGVLFVKAAAHHHHH |
| 153 | mouse ROR1-His | QETELSVSAELVPTSSWNTSSEIDKGSYLTLDEPMNNITTSLGQTAELHCKVSGNPPPSI |
| | (CHO) | RWFKNDAPVVQEPRRISFRATNYGSRLRIRNLDTTDTGYFQCVATNGKKVVSTTGVLFVK |
| | | FGPPPTASPGSSDEYEEDGFCQPYRGIACARFIGNRTVYMESLHMQGEIENQITAAFTMI |
| | | GTSSHLSDKCSQFAIPSLCHYAFPYCDETSSVPKPRDLCRDECEVLENVLCQTEYIFARS |
| | | NPMILMRLKLPNCEDLPQPESPEAANCIRIGIPMADPINKNHKCYNSTGVDYRGTVSVTK |
| | | SGRQCQPWNSQYPHTHSFTALRFPELNGGHSYCRNPGNQKEAPWCFTLDENFKSDLCDIP |
| | | ACDSKDSKEKNKMEHHHHHH |
| 154 | human ROR1-Fc | QETELSVSAELVPTSSWNISSELNKDSYLTLDEPMNNITTSLGQTAELHCKVSGNPPPTI |
| | (CHO) | RWFKNDAPVVQEPRRLSFRSTIYGSRLRIRNLDTTDTGYFQCVATNGKEVVSSTGVLFVK |
| | | FGPPPTASPGYSDEYEEDGFCQPYRGIACARFIGNRTVYMESLHMQGEIENQITAAFTMI |
| | | GTSSHLSDKCSQFAIPSLCHYAFPYCDETSSVPKPRDLCRDECEILENVLCQTEYIFARS |
| | | NPMILMRLKLPNCEDLPQPESPEAANCIRIGIPMADPINKNHKCYNSTGVDYRGTVSVTK |
| | | SGRQCQPWNSQYPHTHTFTALRFPELNGGHSYCRNPGNQKEAPWCFTLDENFKSDLCDIP |
| | | ACDSKDSKEKNKMEGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVMHEA |
| | | LHNHYTQKSLSLSPGK |
| 155 | human ROR2-Fc | EVEVLDPNDPLGPLDGQDGPIPTLKGYFLNFLEPVNNITIVQGQTAILHCKVAGNPPPNV |
| | (CHO) | RWLKNDAPVVQEPRRIIIRKTEYGSRLRIQDLDTTDTGYYQCVATNGMKTITATGVLFVR |
| | | LGPTHSPNHNFQDDYHEDGFCQPYRGIACARFIGNRTIYVDSLQMQGEIENRITAAFTMI |
| | | GTSTHLSDQCSQFAIPSFCHFVFPLCDARSRTPKPRELCRDECEVLESDLCRQEYTIARS |
| | | NPLILMRLQLPKCEALPMPESPDAANCMRIGIPAERLGRYHQCYNGSGMDYRGTASTTKS |
| | | GHQCQPWALQHPHSHHLSSTDFPELGGGHAYCRNPGGQMEGPWCFTQNKNVRMELCDVPS |
| | | CSPRDSSKMGGGGSGGGGSGGGGSDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVMHEALHNHY |
| | | TQKSLSLSPGK |
| 156 | mouse ROR1-Fc | QETELSVSAELVPTSSWNTSSEIDKGSYLTLDEPMNNITTSLGQTAELHCKVSGNPPPSI |
| | (CHO) | RWFKNDAPVVQEPRRISFRATNYGSRLRIRNLDTTDTGYFQCVATNGKKVVSTTGVLFVK |
| | | FGPPPTASPGSSDEYEEDGFCQPYRGIACARFIGNRTVYMESLHMQGEIENQITAAFTMI |
| | | GTSSHLSDKCSQFAIPSLCHYAFPYCDETSSVPKPRDLCRDECEVLENVLCQTEYIFARS |
| | | NPMILMRLKLPNCEDLPQPESPEAANCIRIGIPMADPINKNHKCYNSTGVDYRGTVSVTK |
| | | SGRQCQPWNSQYPHTHSFTALRFPELNGGHSYCRNPGNQKEAPWCFTLDENFKSDLCDIP |
| | | ACDSKDSKEKNKMEGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVMHEA |
| | | LHNHYTQKSLSLSPGK |
| 157 | rat ROR1-Fc | QETELSVSAELVPTSSWNTSSEIDKDSYLTLDEPMNNITTSLGQTAELHCKVSGNPPPNI |
| | (CHO) | RWFKNDAPVVQEPRRISFRATNYGSRLRIRNLDTTDTGYFQCVATSGKKVVSTTGVLFVK |
| | | FGPPPTASPGSSDEYEEDGFCQPYRGIACARFIGNRTVYMESLHMQGEIENQITAAFTMI |
| | | GTSSHLSDKCSQFAIPSLCHYAFPYCDETSSVPKPRDLCRDECEVLENVLCHTEYIFARS |
| | | NPMILMRLKLPNCEDLPQPESPEAANCIRIGIPMADPINKNHKCYNSTGVDYRGTVSVTK |
| | | SGRQCQPWNSQYPHTHSFTALRFPELNGGHSYCRNPGNQKEAPWCFTLDENFKSDLCDIP |
| | | ACDSKDSKEKNKMEGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVMHEA |
| | | LHNHYTQKSLSLSPGK |

A highly interesting class of DNA intercalating toxins for use as payloads for drug conjugates are anthracyclines, because of their proven clinical validation as chemotherapeutic drugs in cancer therapy.

Stability of chemically-conjugated protein drug conjugates is an important consideration, since unintended release of a highly potent anthracycline toxin, like PNU-159682, in the circulation of a patient prior to targeting of the tumour cells would lead to off target effects and undesirable side effects. Some example molecules released from PNU conjugates include release of PNU159682 derivative from different Val-Cit-PAB containing drug linkers.

Potent toxins that can be linked to targeting proteins with high stability are therefore required in order to avoid, or at least reduce, unwanted side effects. Alternatively, linker payloads are designed such that extracellular cleavage releases derivatives of the payload with attenuated potency. However, sufficient potency needs to be retained in order to avoid any reduction in side effect being negated due to the need to administer higher doses to achieve efficacy.

Ease of conjugation is an important factor in producing easily manufacturable products. Payloads of the present disclosure may use a maleimide group, which can react to any available thiol group on a conjugation partner using straightforward and standard conditions. Furthermore, the use of maleimide/thiol chemistry for conjugation allows for site-specific conjugation to introduced thiol groups, for example on the side-chain of an engineered cysteine residue in a protein sequence. In some cases described herein, a cysteine may be introduced via the introduction of his-myc tag containing an engineered cysteine (example sequences include, but are not limited to, QACKAHHHHHHGAEFEQKLISEEDL (SEQ ID NO: 97) or QACGAHHHHHHGAEFEQKLISEEDL (SEQ ID NO: 99)) at the C- or N-terminal of a protein.

Antibody/protein drug conjugates generated using non-selective labelling methods, such as through reaction with amino functionalities within proteins, deliver products containing multiple different species with differing drug to antibody ratios. This impacts the properties of the conjugate including potency and PK properties which impacts in vivo efficacy and toxicities. Therefore, thiol reactive payloads are of great importance, as these can be reacted in high yield, in a simple process, with naturally occurring cysteine residues in proteins or with a cysteine residue engineered into a specific site at any point within the sequence of proteins using molecular biology/recombinant protein expression or chemical synthesis or through chemical modification of expressed, synthetic or natural proteins. In some cases described herein, the cysteine is engineered into the Fc region of an Fc fusion protein.

The present disclosure provides anthracycline (PNU) derivatives suitable for use in drug conjugates. Specifically, derivatives of PNU159682 are provided, which lack the C14 carbon and attached hydroxyl functionality, and are functionalised with an ethylenediamino (EDA) group at the C13 carbonyl of PNU159682. This EDA-PNU159682 can in turn be functionalised, through the amino group of the EDA moiety, with a maleimide containing linker. A maleimide group is present in the anthracycline (PNU) derivatives of formula (V) and may also be present in the anthracycline (PNU) derivatives of formula (VI). Such payloads are able to react with a free thiol group on another molecule. Where the free thiol is on a protein, a protein-drug conjugate (PDC) may be formed.

Surprisingly, derivatives of PNU159682 functionalised with an ethylenediamino (EDA) group and linked to a thiol group via a maleimide group show higher stability compared to non-EDA payloads or liberated payload derivatives with slightly less potency. More stable payloads may be advantageous because of reduced off-target effects, which in turn may lead to reduced side effects and increased patient compliance.

PCT/EP2020/067210 describes anthracycline (PNU) derivatives of formula (V):

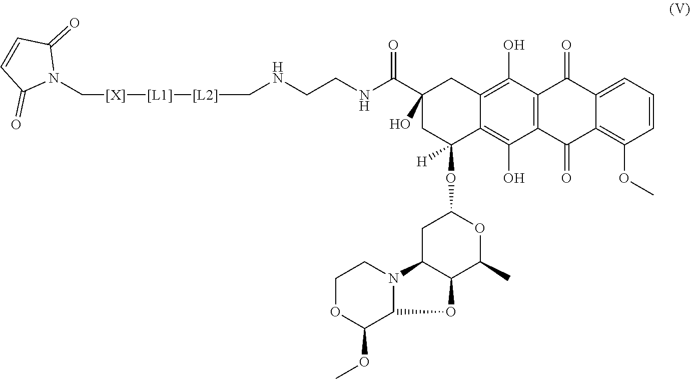

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting ofvaline (Val), citrulline (Cit), alanine (Ala), asparagine (Asn), a peptide, —(CH2)ₙ—, —(CH2CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof.

The anthracycline (PNU) derivative of formula (V) may comprise [L1], [L2] or [L1] and [L2].

Preferably, where [L1] and/or [L2] are peptides, said peptides do not contain glycine.

It will be clear to those of skill in the art that when optional spacers and/or optional linkers are absent a bond remains in their place.

Preferably, [X] is selected from the group comprising polyethylene glycol,

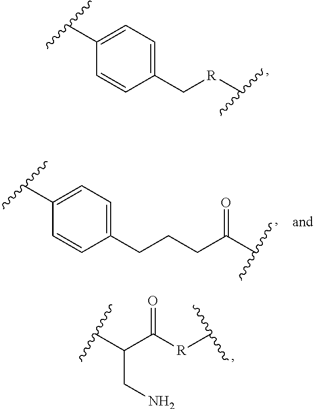

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [L2] is p-aminobenzyloxycarbonyl (PAB) or Alanine.

Preferably, the anthracycline (PNU) derivative comprises [L1] and/or [L2] and [X] is optional. Accordingly, [L1] and/or [L2] may be linkers selected from the group consisting of valine (Val), citrulline (Cit), alanine (Ala), asparagine (Asn), a peptide, —(CH2)ₙ—, —(CH2CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof. The anthracycline (PNU) derivative of formula (V) may comprise [L1], [L2] or [L1] and [L2]. The anthracycline (PNU) derivative of formula (V) may comprise [L1] and/or [L2].

PCT/EP2020/067210 describes anthracycline (PNU) derivatives of formula (V):

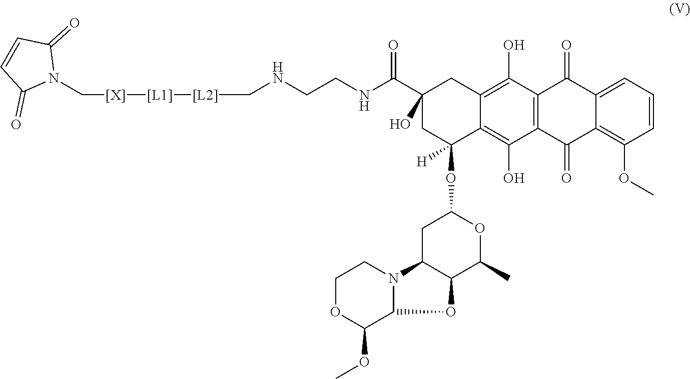

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and/or [L2] are linkers selected from the group consisting of valine (Val), citrulline (Cit), alanine (Ala), asparagine (Asn), a peptide, —(CH2)ₙ—, —(CH2CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof;
wherein the anthracycline (PNU) derivative of formula (V) comprises [L1], [L2] or [L1] and [L2].

Preferably, [X] is selected from the group comprising polyethylene glycol,

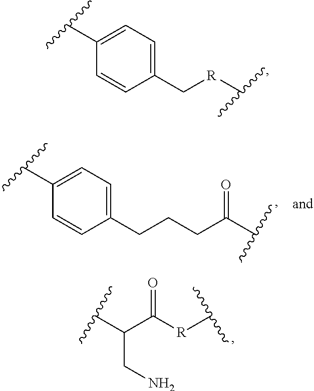

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [L2] is p-aminobenzyloxycarbonyl (PAB) or Alanine.

Preferably, the PNU derivative has a structure selected from:

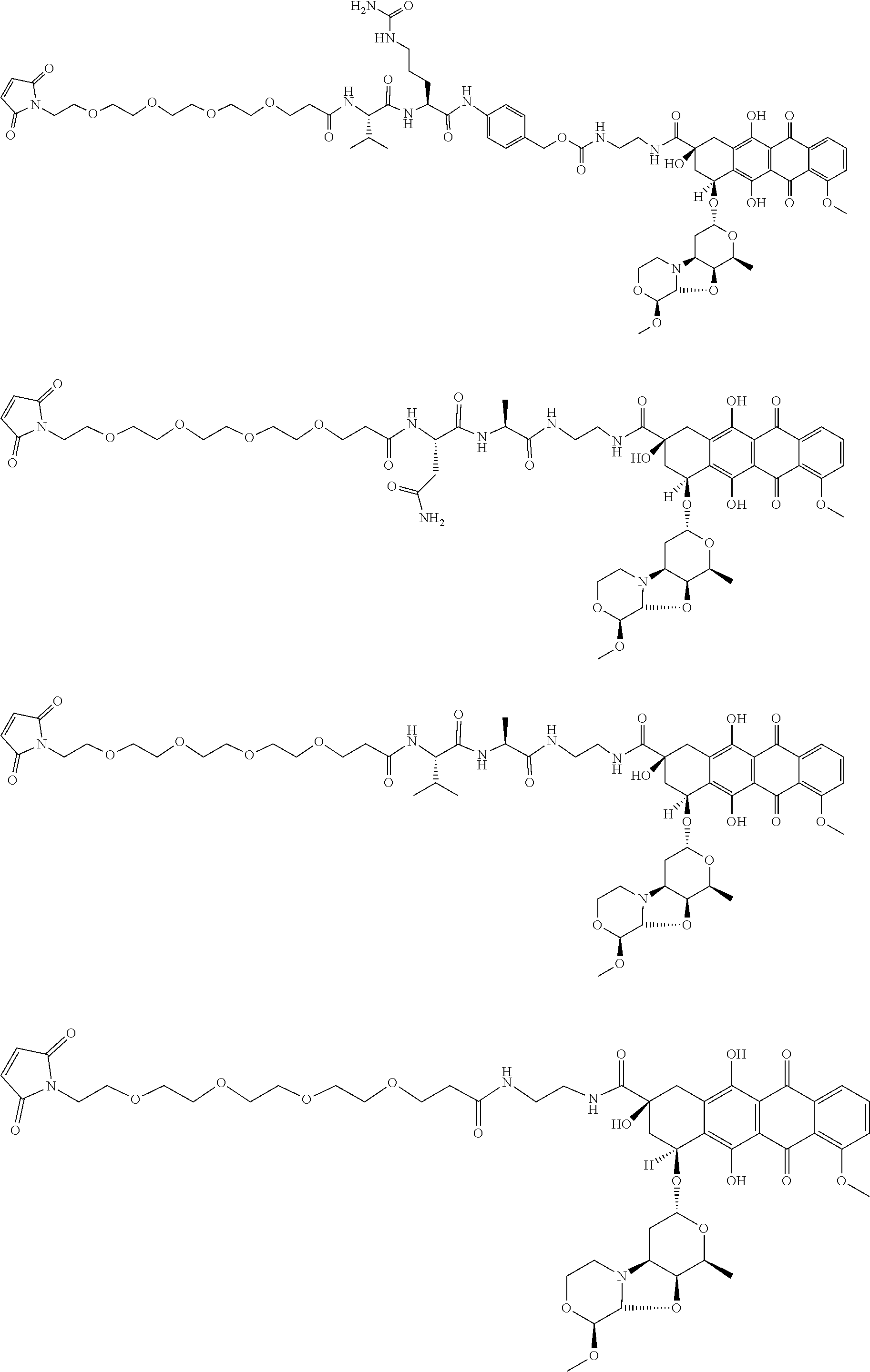

PCT/EP2020/067210 also describes anthracycline (PNU) derivatives of formula (VI):

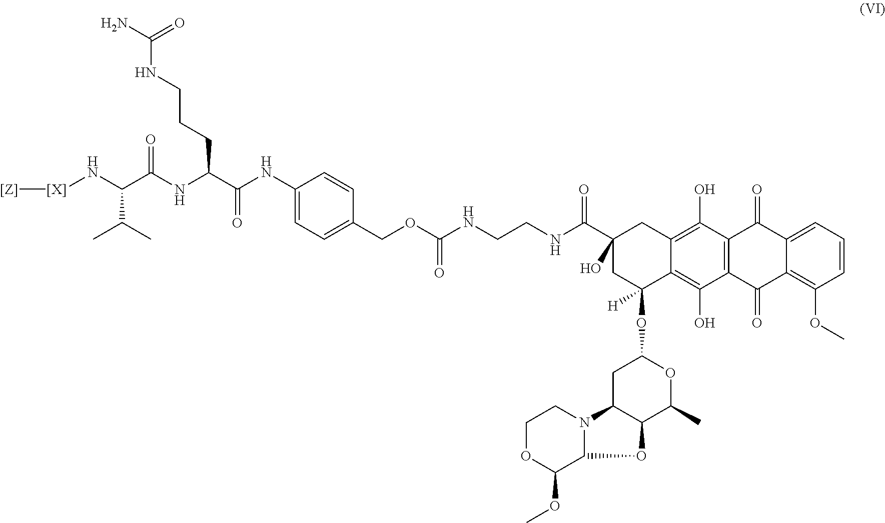

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
wherein [Z] is a reactive group. The reactive group may be any reactive group suitable for use in a conjugation reaction, particularly a conjugation reaction to a target binding molecule. [Z] may therefore be a moiety comprising a functional group for use in bioconjugation reactions.

Functional groups for use in bioconjugation reactions include but are not limited to,
maleimides or alkyl halides for reaction with thiol groups or selenol groups on proteins through thioether and selonoether reactions;
sulphydryl groups for reaction with maleimide, alkyl halide or thiol functionalised molecules including the thiol groups of protein cysteine residues;
activated disulphides such as pyridyl dithiols (Npys thiols) or TNB thiols (5-thiol-2-nitrobenzoic acid) for reaction with thiol groups to form disulphide linkages through thiol disulphide exchange;
amino groups for attachment to carboxyl groups on proteins and biomolecules through amide bond forming reactions;
alkyne groups, particularly ring constrained alkynes such as dibenzocyclooctyne (DBCO) or bicyclo[6.1.0] nonyne (BCN) for the reaction with azido functionalised biomolecules through strain promoted alkyne-azide cycloaddition copper free chemistry. Azido functionalities can be introduced into proteins through, for example, the incorporation of the unnatural amino acid para-azidomethy-L-phenyalanine or into protein glycans using enzyme mediated glycoengineering to attach azido-containing sugar analogues;
azido groups for reaction with alkyne functionalised target-binding molecule through strain promoted alkyne-azide cycloaddition copper free chemistry;
aminoxy groups for reactions with aldehyde and ketone groups on biomolecules through oxime forming ligations. Ketones can be introduced into proteins through the use of amber stop codon technologies such as the incorporation of the non-natural amino acid, para-acetyl phenylalanine. Aldehydes can be found on biomolecules through the presence of reducing sugars and can be introduced into proteins through periodate oxidation of N-terminal serine residues or periodate oxidation of cis-glycol groups of carbohydrates. Aldehyde groups can also be incorporated into proteins through the conversion of protein cysteines, within specific sequences, to formyl glycine by formylglycine generating enzyme. In addition formylglycine containing proteins have been conjugation to payloads via the Hydrazino-Pictet-Spengler (HIPS) ligation;
aldehyde or ketone groups for the reaction with aminoxy or hydrazide or hydrazinyl functionalized biomolecules through oxime or hydrazine bond forming ligation reactions. Protein aminoxy and hydrazide functionalized proteins can be generated through cleavage of intein-fusion proteins.
[Z] may therefore be selected from the group consisting of a maleimide, an alkyl halide, a sulphydryl group, an activated disulphide (such as pyridyl dithiols (Npys thiols) or TNB thiols (5-thiol-2-nitrobenzoic acid)), an amino group, an alkyne group (such as ring constrained alkynes such as dibenzocyclooctyne (DBCO) or bicyclo[6.1.0] nonyne (BCN)), an azido group, an aminoxy group, an aldehyde group and a ketone group.
[Z] may also be a moiety for enzyme mediated bioconjugation reactions. Moieties for use in enzyme mediated conjugation reactions include but are not limited to polyGly [(Gly)ₙ] for use in sortase-enzyme mediated antibody conjugation or an appropriate primary amine for bacterial transglutaminase mediated conjugation to glutamine γ-carboxyamide groups contained with sequences such as Lys-Lys-Gln-Gly and Lys-Pro-Glu-Thr-Gly.
[Z] may therefore be selected from the group consisting of polyGly and a primary amine.

The PNU derivative according to formula (VI) may therefore correspond to a PNU derivative of formula (V) wherein L1 is Val-Cit-PAB, L2 is absent and wherein the maleimide group may be replaced with another Reactive Group as defined above.

Preferably, [X] is selected from the group comprising polyethylene glycol,

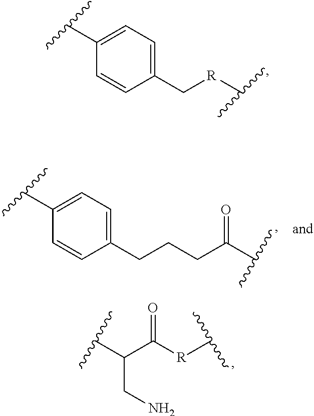

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

The PNU derivative according to formula (V) or formula (VI) may be conjugated to a ROR1 specific antigen binding molecule according to the present invention or to a recombinant fusion protein or recombinant fusion protein dimer of the invention.

According to a tenth aspect, the invention provides a target-binding molecule-drug conjugate, comprising
(a) a ROR1 specific antigen binding molecule according to the first, second or ninth aspect, or a recombinant fusion protein or recombinant fusion protein of the third or fourth aspect or a recombinant fusion protein dimer of the fifth aspect, and
(b) an anthracycline (PNU) derivative,
wherein the target-binding molecule-drug conjugate has the structure of formula (III):

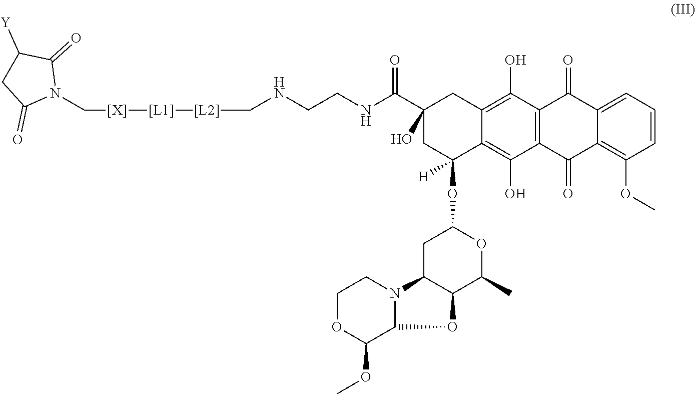

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting ofvaline (Val), citrulline (Cit), alanine (Ala), asparagine (Asn), a peptide, —(CH2)ₙ—, —(CH2CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises a ROR1 specific antigen binding molecule according to the first, second or ninth aspect, or a recombinant fusion protein or recombinant fusion protein of the third or fourth aspect or a recombinant fusion protein dimer of the fifth aspect.

The target-binding molecule-drug conjugate of formula (III) may comprise [L1], [L2] or [L1] and [L2].

Preferably, target-binding molecule-drug conjugate where [L1] and/or [L2] are peptides, said peptides do not contain glycine.

It will be clear to those of skill in the art that when optional spacers and/or optional linkers are absent a bond remains in their place.

Preferably, the target-binding molecule-drug conjugate has a structure selected from:

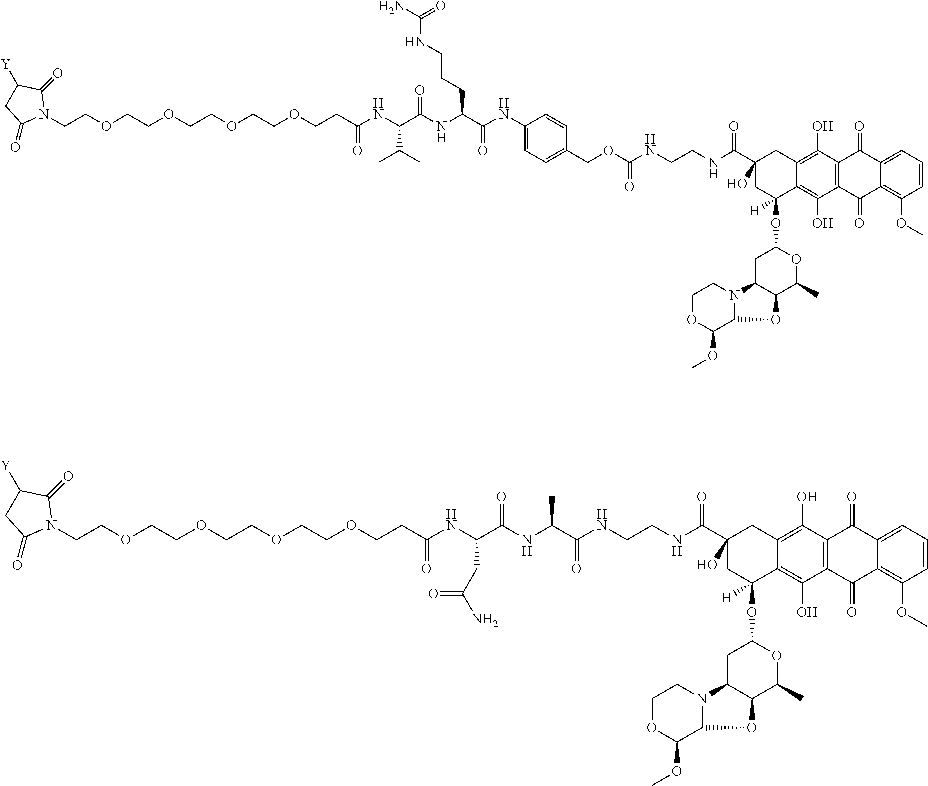

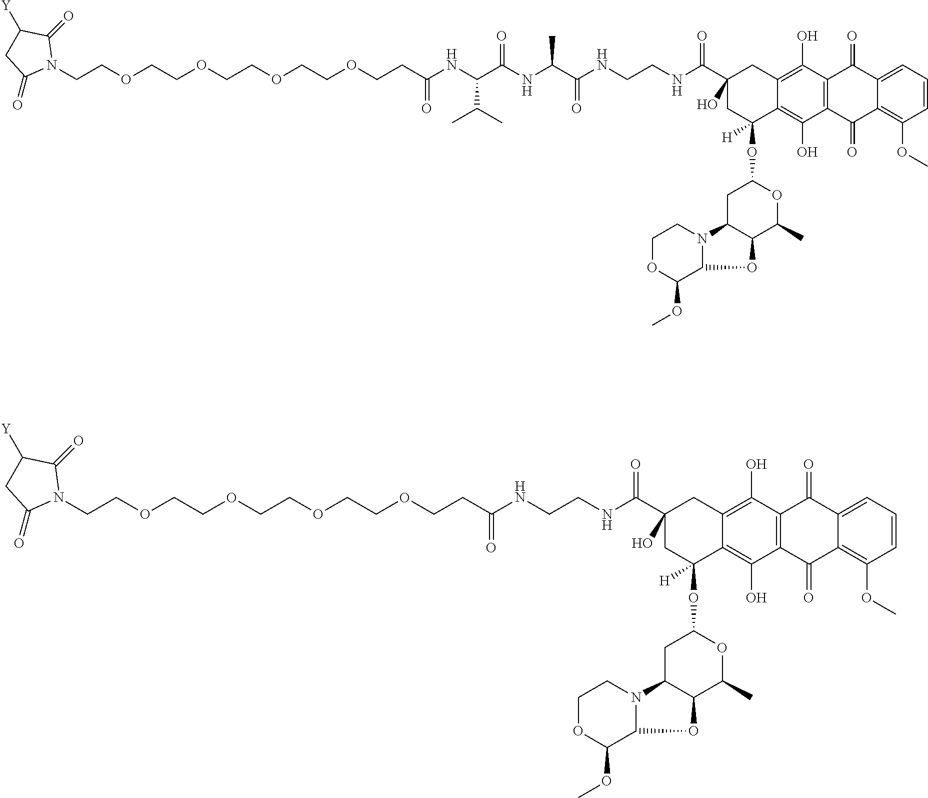

According to an eleventh aspect, the invention provides a target-binding molecule-drug conjugate, comprising
(a) a ROR1 specific antigen binding molecule according to the first, second or ninth aspect, or a recombinant fusion protein or recombinant fusion protein dimer according to the third, fourth or fifth aspect, and
(b) an anthracycline (PNU) derivative, wherein the target-binding molecule-drug conjugate has the structure of formula (IV):

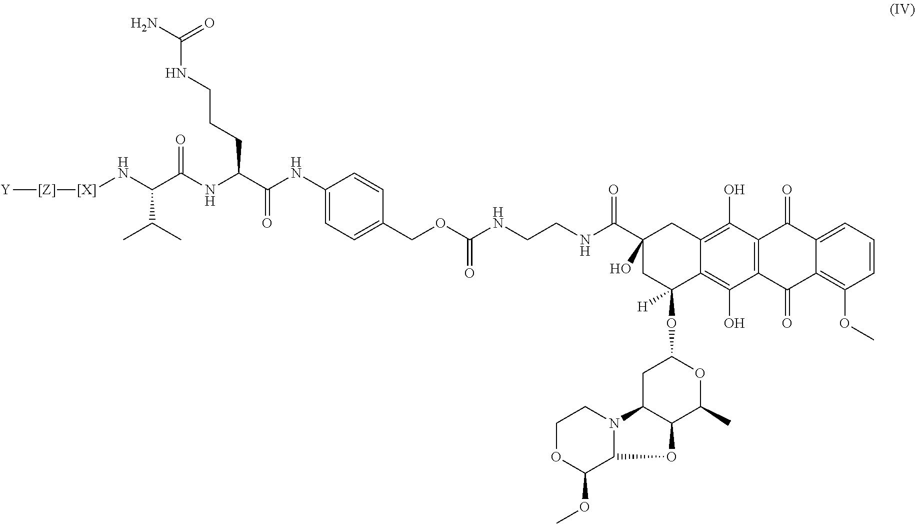

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y comprises a ROR1 specific antigen binding molecule according to the first, second or ninth aspect, or a recombinant fusion protein or recombinant fusion protein dimer according to the third, fourth or fifth aspect.
[Z] is a typically a moiety derived from a reactive group used to conjugate the anthracycline (PNU) derivative and the target-binding molecule. [Z] may be a moiety derived from a reactive group selected from the group consisting of a maleimide, an alkyl halide, a sulphydryl group, an activated disulphide, an amino group, an alkyne group, an azido group, an aminoxy group, an aldehyde group and a ketone group.
[Z] may therefore be selected from the group consisting of a disulphide bond, an amide bond, an oxime bond, a hydrazone bond, a thioether bond, a 1, 2, 3 triazole and polyGly.

Preferably, [X] is selected from the group comprising polyethylene glycol,

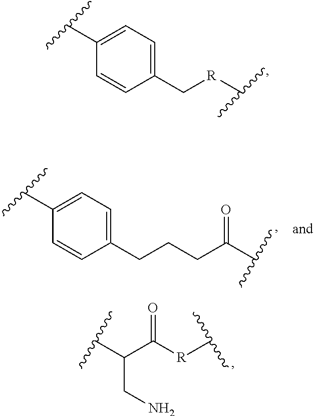

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, the target-binding molecule is a protein or a nucleic acid. Examples of target-binding proteins (which may also be referred to as specific antigen binding proteins) include but are not limited to an immunoglobulin or antibody, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), a scFv-Fc, (scFv)₂, a diabody, a triabody, a tetrabody, a bispecific t-cell engager, an intein, a VNAR domain, a single domain antibody (sdAb), a VH domain, a scaffold protein (affibodies, centyrins, darpins etc.). Examples of target-binding nucleic acids include but are not limited to aptamers.

Preferably, the target-binding molecule-drug conjugate is a protein and the anthracycline (PNU) derivative is conjugated to a thiol-containing amino acid residue in the amino acid sequence of a protein or to a thiol group introduced by chemical modification of the protein, for example incorporated at the N-terminus or C-terminus of the amino acid sequence of the specific antigen binding protein. Thiol groups may also be introduced into other target-binding molecules, such as nucleic acids.

In one embodiment of the tenth or eleventh aspect, the target-binding molecule-drug conjugate, Y comprises a ROR1 specific antigen binding molecule according to the first or second aspects of the invention, conjugated to the PNU derivative via a human immunoglobulin Fc region or fragment thereof.

In one embodiment the fragment of the human immunoglobulin Fc region may be selected from the group consisting of an Fc heavy chain, a CH2 region and a CH3 region.

Also provided herein is the target-binding molecule-drug conjugate according to the above aspects, for use in therapy.

Also provided herein is the target-binding molecule-drug conjugate according to the above aspects, for use in the treatment of cancer.

Also provided herein is the use of a target-binding molecule-drug conjugate according to the above aspects in the manufacture of a medicament for the treatment of a disease in a patient in need thereof.

Also provided herein is a method of treatment of a disease in a patient in need of treatment comprising administration to said patient of a therapeutically effective dosage of a target-binding molecule-drug conjugate according to the above aspects. The disease may be cancer.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer. The cancer may be mesothelioma or triple negative breast cancer (TNBC). The mesothelioma may be pleural mesothelioma.

Also provided herein is a pharmaceutical composition comprising a target-binding molecule-drug conjugate according to any of the above aspects, and at least one other pharmaceutically acceptable ingredient.

### Definitions

An antigen specific binding molecule of the invention comprises amino acid sequence derived from a synthetic library of VNAR molecules, or from libraries derived from the immunization of a cartilaginous fish. The terms VNAR, IgNAR and NAR may be used interchangeably also.

Amino acids are represented herein as either a single letter code or as the three letter code or both.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “Complementarity Determining Regions” or CDRs (i.e., CDR1 and CDR3) refers to the amino acid residues of a VNAR domain the presence of which are typically involved in antigen binding. Each VNAR typically has two CDR regions identified as CDR1 and CDR3. Additionally, each VNAR domain comprises amino acids from a “hypervariable loop” (HV), which may also be involved in antigen binding. In some instances, a complementarity determining region can include amino acids from both a CDR region and a hypervariable loop. In other instances, antigen binding may only involve residues from a single CDR or HV. According to the generally accepted nomenclature for VNAR molecules, a CDR2 region is not present.

“Framework regions” (FW) are those VNAR residues other than the CDR residues. Each VNAR typically has five framework regions identified as FW1, FW2, FW3a, FW3b and FW4.

The boundaries between FW, CDR and HV regions in VNARs are not intended to be fixed and accordingly some variation in the lengths and compositions of these regions is to be expected. This will be understood by those skilled in the art, particularly with reference to work that have been carried out in analyzing these regions. (Anderson et al., PLoS ONE (2016) 11 (8); Lui et al., Mol Immun (2014) 59, 194-199; Zielonka et al., Mar Biotechnol (2015). 17, (4) 386-392; Fennell et al., J Mol Biol (2010) 400. 155-170; Kovalenko et al., J Biol Chem (2013) 288. 17408-17419; Dooley et al., (2006) PNAS 103 (6). 1846-1851). The molecules of the present invention, although defined by reference to FW, CDR and HV regions herein, are not limited to these strict definitions. Variation in line with the understanding in the art as the structure of the VNAR domain is therefore expressly contemplated herein.

A “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein.

A codon set is typically represented by 3 capital letters in italics, e.g. NNK, NNS, XYZ, DVK etc. A “non-random codon set” therefore refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al.; J. Mol. Biol. (1999), 296, 57-86); Garrard & Henner, Gene (1993), 128, 103). Such sets of oligonucleotides having certain codon sets can be synthesized using commercial nucleic acid synthesizers (available from, for example, Applied Biosystems, Foster City, CA), or can be obtained commercially (for example, from Life Technologies, Rockville, MD). A set of oligonucleotides synthesized having a particular codon set will typically include a plurality of oligonucleotides with different sequences, the differences established by the codon set within the overall sequence. Oligonucleotides used according to the present invention have sequences that allow for hybridization to a VNAR nucleic acid template and also may where convenient include restriction enzyme sites.

“Cell”, “cell line”, and “cell culture” are used interchangeably (unless the context indicates otherwise) and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, etc. Eukaryotic cells use control sequences such as promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “fusion protein” in this text means, in general terms, one or more proteins joined together by chemical means, including hydrogen bonds or salt bridges, or by peptide bonds through protein synthesis or both. Typically fusion proteins will be prepared by DNA recombination techniques and may be referred to herein as recombinant fusion proteins.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from an allogenic or xenogenic source. The DNA may, optionally, include marker or selection genes, for example, antibiotic resistance genes, temperature resistance genes, etc.

A “highly diverse position” refers to a position of an amino acid located in the variable regions of the light and heavy chains that have a number of different amino acid represented at the position when the amino acid sequences of known and/or naturally occurring antibodies or antigen binding fragments are compared. The highly diverse positions are typically in the CDR or HV regions.

“Identity” describes the relationship between two or more polypeptide sequences or two or more polynucleotide sequences, as determined by comparing the sequences. Identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. (1990) 215, 403).

Preferably, the amino acid sequence of the protein has at least 45% identity, using the default parameters of the BLAST computer program (Atschul et al., J. Mol. Biol. (1990) 215, 403-410) provided by HGMP (Human Genome Mapping Project), at the amino acid level, to the amino acid sequences disclosed herein.

More preferably, the protein sequence may have at least 45%, 46%, 47%, 48%, 49%, 50%, 55%, 60%, 65%, 66%, 67%, 68%, 69%, 70%, 75%, 80%, 85%, 90% and still more preferably 95% (still more preferably at least 96%, 97%, 98% or 99%) identity, at the nucleic acid or amino acid level, to the amino acid sequences as shown herein.

The protein may also comprise a sequence which has at least 45%, 46%, 47%, 48%, 49%, 50%, 50%, 55%, 60%, 65%, 66%, 67%, 68%, 69%, 70%, 75%, 80%, 85%, 90%, 95%, 96%, 97%, 98%, or 99% identity with a sequence disclosed herein, using the default parameters of the BLAST computer program provided by HGMP, thereto

A “library” refers to a plurality of VNARs or VNAR fragment sequences (for example, polypeptides of the invention), or the nucleic acids that encode these sequences, the sequences being different in the combination of variant amino acids that are introduced into these sequences according to the methods of the invention.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

A “mutation” is a deletion, insertion, or substitution of a nucleotide(s) relative to a reference nucleotide sequence, such as a wild type sequence.

“Natural” or “naturally occurring” VNARs, refers to VNARs identified from a non-synthetic source, for example, from a tissue source obtained ex vivo, or from the serum of an animal of the Elasmobranchii subclass. These VNARs can include VNARs generated in any type of immune response, either natural or otherwise induced. Natural VNARs include the amino acid sequences, and the nucleotide sequences that constitute or encode these antibodies. As used herein, natural VNARs are different than “synthetic VNARs”, synthetic VNARs referring to VNAR sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promotor or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

The term “protein” means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues, variants and derivatives of a protein wherein the fragment, analogue, variant or derivative retains essentially the same biological activity or function as a reference protein. Examples of protein analogues and derivatives include peptide nucleic acids, and DARPins (Designed Ankyrin Repeat Proteins).

A fragment, analogue, variant or derivative of the protein may be at least 25 preferably 30 or 40, or up to 50 or 100, or 60 to 120 amino acids long, depending on the length of the original protein sequence from which it is derived. A length of 90 to 120, 100 to 110 amino acids may be convenient in some instances.

The fragment, derivative, variant or analogue of the protein may be (i) one in which one or more of the amino acid residues are substituted with a conserved or non-conserved amino acid residue (preferably, a conserved amino acid residue) and such substituted amino acid residue may or may not be one encoded by the genetic code, or (ii) one in which one or more of the amino acid residues includes a substituent group, or (iii) one in which the additional amino acids are fused to the mature polypeptide, such as a leader or auxiliary sequence which is employed for purification of the polypeptide. Such fragments, derivatives, variants and analogues are deemed to be within the scope of those skilled in the art from the teachings herein.

“Oligonucleotides” are short-length, single- or double-stranded polydeoxynucleotides that are chemically synthesized by known methods (such as phosphotriester, phosphite, or phosphoramidite chemistry, using solid-phase techniques). Further methods include the polymerase chain reaction (PCR) used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation. DNA is “purified” when the DNA is separated from non-nucleic acid impurities (which may be polar, non-polar, ionic, etc.).

A “source” or “template” VNAR, as used herein, refers to a VNAR or VNAR antigen binding fragment whose antigen binding sequence serves as the template sequence upon which diversification according to the criteria described herein is performed. An antigen binding sequence generally includes within a VNAR preferably at least one CDR, preferably including framework regions.

A “transcription regulatory element” will contain one or more of the following components: an enhancer element, a promoter, an operator sequence, a repressorgene, and a transcription termination sequence.

“Transformation” means a process whereby a cell takes up DNA and becomes a “transformant”. The DNA uptake may be permanent or transient. A “transformant” is a cell which has taken up and maintained DNA as evidenced by the expression of a phenotype associated with the DNA (e.g., antibiotic resistance conferred by a protein encoded by the DNA).

A “variant” or “mutant” of a starting or reference polypeptide (for example, a source VNAR or a CDR thereof), such as a fusion protein (polypeptide) or a heterologous polypeptide (heterologous to a phage), is a polypeptide that (1) has an amino acid sequence different from that of the starting or reference polypeptide and (2) was derived from the starting or reference polypeptide through either natural or artificial mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source VNAR or antigen binding fragment) would be a variant polypeptide with respect to a source VNAR or antigen binding fragment. Thus, a variant CDR refers to a CDR comprising a variant sequence with respect to a starting or reference polypeptide sequence (such as that of a source VNAR or antigen binding fragment). A variant amino acid, in this context, refers to an amino acid different from the amino acid at the corresponding position in a starting or reference polypeptide sequence (such as that of a source VNAR or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

A “wild-type” or “reference” sequence or the sequence of a “wild-type” or “reference” protein/polypeptide, such as a coat protein, or a CDR of a source VNAR, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man induced means. The products of such processes are “variant” or “mutant” forms of the original “wild-type” protein or gene.

A “humanised” antigen specific antigen binding molecule may be modified at one or more amino acid sequence position to reduce the potential for immunogenicity in vivo, while retaining functional binding activity for the specific epitopes on the specific antigen.

Humanization of antibody variable domains is a technique well-known in the art to modify an antibody which has been raised, in a species other than humans, against a therapeutically useful target so that the humanized form may avoid unwanted immunological reaction when administered to a human subject. The methods involved in humanization are summarized in Almagro J. C and William Strohl W. Antibody Engineering: Humanization, Affinity Maturation, and Selection Techniques in Therapeutic MonoclonalAntibodies: From Bench to Clinic. Edited by An J. 2009 John Wiley & Sons, Inc and in Strohl W. R. and Strohl L. M., Therapeutic Antibody Engineering, Woodhead Publishing 2012.

Although IgNARs have distinct origins compared to immunoglobulins and have very little sequence homology compared to immunoglobulin variable domains there are some structural similarities between immunoglobulin and IgNAR variable domains, so that similar processes can be applied to the VNAR domain. For example, WO2013/167883, incorporated by reference, provides a description of the humanization of VNARs, see also Kovalenko O. V., et al. J Biol Chem. 2013. 288(24): p. 17408-19.

A humanised antigen specific binding molecule may differ from a wild-type antigen specific binding molecule by substituting one or more framework amino acid residues with a corresponding framework amino acid residue of DPK-9. DPK-9 is a human germline VL scaffold, a member of the variable kappa subgroup 1 (VK1). DPK-9 has a sequence according to:

(SEQ ID NO: 132)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPNTFGQ
GTKVEIK

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of an antigen-specific binding protein, such as a monoclonal antibody or VNAR, onto a T cell, thereby allowing a large number of specific T cells to be generated, for example, for use in adoptive cell therapy. CARs may direct the specificity of the cell to a tumour associated antigen, for example. CARs may comprise an intracellular activation domain, a transmembrane domain, and an extracellular domain comprising a tumour associated antigen binding region. In particular aspects, CARs comprise fusions of single-chain variable fragments (scFv) derived from monoclonal antibodies fused to CD3-zeta transmembrane and endodomains. In other particular aspects, CARs comprise fusions of the VNAR domains described herein with CD3-zeta transmembrane and endodomains. The specificity of other CAR designs may be derived from ligands of receptors (e.g., peptides) or from pattern-recognition receptors, such as Dectins. In particular embodiments, one can target malignant B cells by redirecting the specificity of T cells by using a CAR specific for the B-lineage molecule, CD 19. In certain cases, the spacing of the antigen-recognition domain can be modified to reduce activation-induced cell death. In certain cases, CARs comprise domains for additional co-stimulatory signalling, such as CD3-zeta, FcR, CD27, CD28, CD 137, DAP 10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “conjugation” as used herein may refer to any method of chemically linking two or more chemical moieties. Typically, conjugation will be via covalent bond. In the context of the present invention, at least one of the chemical moieties will be a polypeptide and in some cases the conjugation will involve two or more polypeptides, one or more of which may be generated by recombinant DNA technology. A number of systems for conjugating polypeptides are known in the art. For example, conjugation can be achieved through a lysine residue present in the polypeptide molecule using N-hydroxy-succinimide or through a cysteine residue present in the polypeptide molecule using maleimidobenzoyl sulfosuccinimide ester. In some embodiments, conjugation occurs through a short-acting, degradable linkage including, but not limited to, physiologically cleavable linkages including ester, carbonate ester, carbamate, sulfate, phosphate, acyloxyalkyl ether, acetal, and ketal, hydrazone, oxime and disulphide linkages. In some embodiments linkers that are cleavable by intracellular or extracellular enzymes, such as cathepsin family members, cleavable under reducing conditions or acidic pH are incorporated to enable releases of conjugated moieties from the polypeptide or protein to which it is conjugated.

A particularly preferred method of conjugation is the use of intein-based technology (US2006247417) Briefly, the protein of interest is expressed as an N terminal fusion of an engineered intein domain (Muir 2006 Nature 442, 517-518). Subsequent N to S acyl shift at the protein-intein union results in a thioester linked intermediate that can be chemically cleaved with bis-aminoxy agents or amino-thiols to give the desired protein C-terminal aminoxy or thiol derivative, respectively. These C-terminal aminoxy and thiol derivatives can be reacted with aldehyde/ketone and maleimide functionalised moieties, respectively, in a chemoselective fashion to give the site-specific C-terminally modified protein.

In another preferred method of conjugation the VNARs are directly expressed with an additional cysteine at or near the C-terminal region of the VNAR or incorporated within a short C-terminal tag sequence enabling conjugation with thiol reactive payloads such as maleimide functionalised moieties.

Conjugation as referred to herein is also intended to encompass the use of a linker moiety, which may impart a number of useful properties. Linker moieties include, but are not limited to, peptide sequences such as poly-glycine, gly-ser, val-cit or val-ala. In certain cases, the linker moiety may be selected such that it is cleavable under certain conditions, for example via the use of enzymes, nucleophilic/basic reagents, reducing agents, photo-irradiation, electrophilic/acidic reagents, organometallic and metal reagents, or oxidizing reagents, or the linker may be specifically selected to resist cleavage under such conditions.

Polypeptides may be conjugated to a variety of functional moieties in order to achieve a number of goals. Examples of functional moieties include, but are not limited to, polymers such as polyethylene glycol in order to reduce immunogenicity and antigenicity or to improve solubility. Further non-limiting examples include the conjugation of a polypeptide to a therapeutic agent or a cytotoxic agent.

The term “detectable label” is used herein to specify that an entity can be visualized or otherwise detected by spectroscopic, photochemical, biochemical, immunochemical, electrical, optical, chemical or other means. The detectable label may be selected such that it generates a signal which can be measured and whose intensity is proportional to the amount of bound entity. A wide variety of systems for labelling and/or detecting proteins and peptides are known in the art. A label may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionuclides, fluorophores, chemiluminescent agents, microparticles, enzymes, colorimetric labels, magnetic labels, haptens, molecular beacons, and aptamer beacons.

Methods of killing or inhibiting the growth of a cells expressing ROR1 in vitro or in a patient are contemplated herein, in general, the term “killing” as used herein in the context of cells means causing a cell death. This may be achieved by a number of mechanisms, such as necrosis or other cells injury, or the induction of apoptosis. The phrases “inhibiting the growth” or “inhibiting proliferation” when used herein are intended to encompass the prevention of cell development, more specifically the prevention of cell division.

As used herein, an alkyl group is a straight chain or branched, substituted or unsubstituted group (preferably unsubstituted) containing from 1 to 40 carbon atoms. An alkyl group may optionally be substituted at any position. The term “alkenyl,” as used herein, denotes a group derived from the removal of a single hydrogen atom from a straight- or branched-chain aliphatic moiety having at least one carbon-carbon double bond. The term “alkynyl,” as used herein, refers to a group derived from the removal of a single hydrogen atom from a straight- or branched-chain aliphatic moiety having at least one carbon-carbon triple bond.

The term ‘alkyl’, ‘aryl’, ‘heteroaryl’ etc also include multivalent species, for example alkylene, arylene, ‘heteroarylene’ etc. Examples of alkylene groups include ethylene (—CH₂—CH₂—), and propylene (—CH₂—CH₂—CH₂—). An exemplary arylene group is phenylene (—C₆H₄—), and an exemplary heteroarylene group is pyridinylene (—C₅H₃N—).

Aromatic rings are cyclic aromatic groups that may have 0, 1, 2 or more, preferably 0, 1 or 2 ring heteroatoms. Aromatic rings may be optionally substituted and/or may be fused to one or more aromatic or non-aromatic rings (preferably aromatic), which may contain 0, 1, 2, or more ring heteroatoms, to form a polycyclic ring system.

Aromatic rings include both aryl and heteroaryl groups. Aryl and heteroaryl groups may be mononuclear, i.e. having only one aromatic ring (like for example phenyl or phenylene), or polynuclear, i.e. having two or more aromatic rings which may be fused (like for example napthyl or naphthylene), individually covalently linked (like for example biphenyl), and/or a combination of both fused and individually linked aromatic rings. Preferably the aryl or heteroaryl group is an aromatic group which is substantially conjugated over substantially the whole group. Aryl groups may contain from 5 to 40 ring carbon atoms, from 5 to 25 carbon atoms, from 5 to 20 carbon atoms, or from 5 to 12 carbon atoms. Heteroaryl groups may be from 5 to 40 membered, from 5 to 25 membered, from 5 to 20 membered or from 5 to 12 membered rings, containing 1 or more ring heteroatoms selected from N, O, S and P. An aryl or heteroaryl may be fused to one or more aromatic or non-aromatic rings (preferably an aromatic ring) to form a polycyclic ring system.

Aryl and heteroaryl preferably denote a mono-, bi- or tricyclic aromatic or heteroaromatic group with up to 25 ring atoms that may also comprise condensed rings and is optionally substituted.

Preferred aryl groups include, without limitation, benzene, biphenylene, triphenylene, [1,1′:3′,1″ ]terphenyl-2′-ylene, naphthalene, anthracene, binaphthylene, phenanthrene, pyrene, dihydropyrene, chrysene, perylene, tetracene, pentacene, benzopyrene, fluorene, indene, indenofluorene, spirobifluorene, etc.

Preferred heteroaryl groups include, without limitation, 5-membered rings like pyrrole, pyrazole, silole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6-membered rings like pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, and fused systems like carbazole, indole, isoindole, indolizine, indazole, benzimidazole, benzotriazole, purine, naphthimidazole, phenanthrimidazole, pyridimidazole, pyrazin-imidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, dithienopyridine, isobenzothiophene, dibenzothiophene, benzothiadiazothiophene, 2,5-dihydropyrrolo[3,4-c]pyrrol-1,4-dione (diketopyrrolopyrrole, DPP), 2-oxo-1 H-indol-3-ylidene, [3,3′-bipyrrolo[2,3-b]pyridinylidene]-2,2′(1 H,1′H)-dione (pyridine isoindigo) and (3E)-3-(2-oxo-1 H-indol-3-ylidene)-1 H-indol-2-one (isoindigo), or combinations thereof. The heteroaryl groups may be substituted with alkyl, alkoxy, thioalkyl, fluoro, fluoroalkyl or further aryl or heteroaryl substituents. Preferably a heteroaryl group is thiophene.

Particularly preferred heteroatoms are selected from O, S, N, P and Si. Typically, hydrogen will complete the valency of a heteroatom included in the molecules of the invention, e.g. for N there may be —NH— or —NH₂ where one or two other groups are involved.

As used herein, the term “optionally substituted” means that one or more of the hydrogen atoms in the optionally substituted moiety is replaced by a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable compounds. The term “stable”, as used herein, refers to compounds that are chemically feasible and can exist for long enough at room temperature (i.e. 16-25° C.) to allow for their detection, isolation and/or use in chemical synthesis.

Any of the above groups (for example, those referred to herein as “optionally substituted”, including alkyl, aryl and heteroaryl groups) may optionally comprise one or more substituents, preferably selected from silyl, sulfo, sulfonyl, formyl, amino, imino, nitrilo, mercapto, cyano, nitro, halogen, —NCO, —NCS, —OCN, —SCN, —C(═O)NR⁰R⁰⁰, —C(═O)X⁰, —C(═O)R⁰, —NR⁰R⁰⁰, C₁₋₁₂alkyl, C₁₋₁₂alkenyl, C₁₋₁₂alkynyl, C₆-₁₂aryl, C₃₋₁₂cycloalkyl, heterocycloalkyl having 4 to 12 ring atoms, heteroaryl having 5 to 12 ring atoms, C₁₋₁₂ alkoxy, hydroxy, C₁₋₁₂ alkylcarbonyl, C₁₋₁₂ alkoxy-carbonyl, C₁₋₁₂ alkylcarbonyloxy or C₁₋₁₂ alkoxycarbonyloxy wherein one or more H atoms are optionally replaced by F or Cl and/or combinations thereof; wherein X⁰ is halogen and R⁰ and R⁰⁰ are, independently, H or optionally substituted C₁₋₁₂alkyl. The optional substituents may comprise all chemically possible combinations in the same group and/or a plurality of the aforementioned groups (for example amino and sulfonyl if directly attached to each other represent a sulfamoyl radical). In one embodiment, the substituent is not acyl. As used herein acyl refers to an acyl group which is a moiety derived by the removal of one or more hydroxyl groups from an oxoacid, such as a carboxylic acid. It contains a double-bonded oxygen atom and an alkyl group.

In some embodiments the groups may be unsubstituted. For example, the anthracycline (PNU) derivative may be of formula (V):

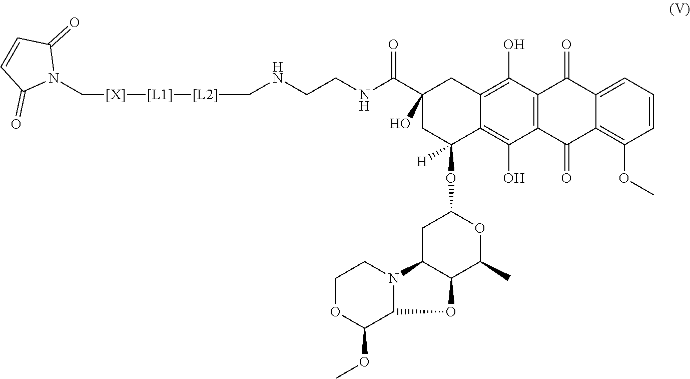

wherein [X] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof;
[L1] and [L2] are optional linkers selected from the group consisting ofvaline (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof.

In embodiments wherein the groups are unsubstituted, [X] is preferably selected from the group comprising polyethylene glycol and

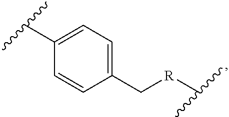

wherein [symbol] represents the point of attachment to the rest of the molecule and wherein [R] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof.

In general, the term PAB is intended to mean p-aminobenzyloxycarbonyl. Occasionally in the literature, the term PAB may be used to indicated p-aminobenzyl. In the present specification, PAB is intended to indicate p-aminobenzyloxycarbonyl.

The term “target-binding molecule” refers to any molecule that binds to a given target. In this context, “target” and “antigen” may be used interchangeably. Examples of target-binding molecules include natural or recombinant proteins including immunoglobulins or antibodies, immunoglobulin Fc regions, immunoglobulin Fab regions, Fab, Fab′, Fv, Fv-Fc, single chain Fv (scFv), scFv-Fc, (scFv)₂, diabodies, triabodies, tetrabodies, bispecific t-cell engagers, inteins, intein fusions, VNAR domains, single domain antibodies (sdAb), VH domains, scaffold proteins (affibodies, centyrins, darpins etc.) and nucleic acids including aptamers or small molecules or natural products that have been developed to bind to the target or naturally bind to the target.

Chemical modification of proteins and biomolecules to introduce thiols is well established. Methods include reaction of amine groups with 2-iminothiolane (Traut's reagent), modification of amine groups with NHS-ester containing heterobifunctional agents such as N-succinimidyl S-acetylthio late (SATA) or N-succinimidyl-4-(2-pyridyldithio)butanoate (SPDB), followed by treatment with hydroxylamine and reducing agents respectively and cleavage of engineered intein-fusion proteins with cysteamine to generate C-terminal thiol proteins and peptides.

The phrase “selected from the group comprising” may be substituted with the phrase “selected from the group consisting of” and vice versa, wherever they occur herein.

The PNU derivatives described herein may be prepared accordingly to standard synthesis methods. Mass spectrometry may be used to verify that the correct molecules have been produced (Table 4).

TABLE 4
Characterisation of PNU derivatives by mass spectrometry

| | | Exp. | Obs. |
|---|---|---|---|
| | | mass, | mass, |
| Name | Structure | Da | Da |
| MA-PEG4- vc-PAB- EDA- PNU159682 | 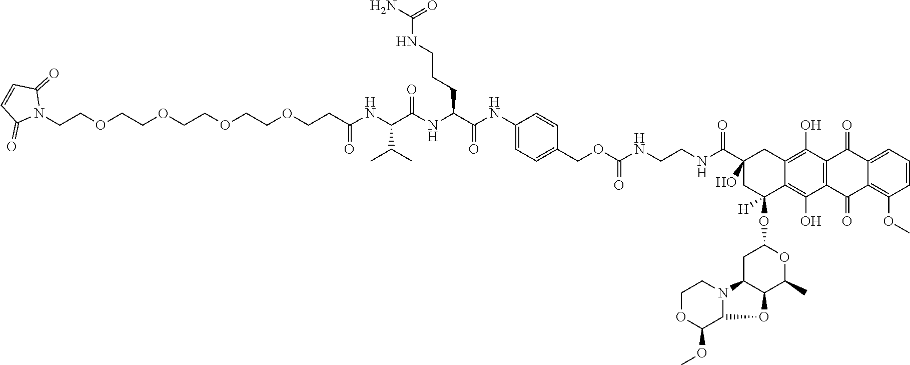 | 1402.5 | 1402.1 |
| MA-PEG4- va-EDA- PNU159682 | 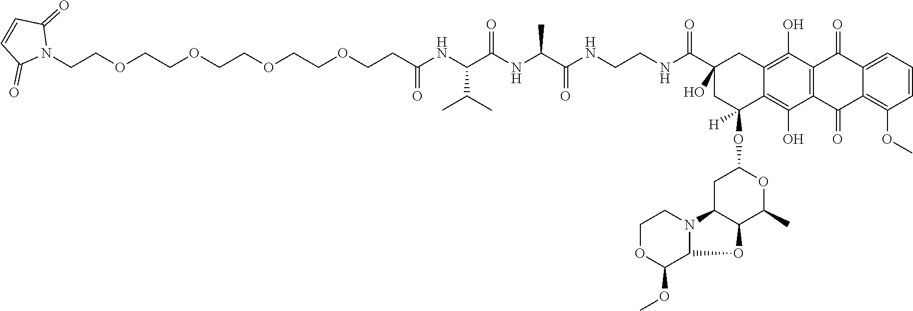 | 1167.2 | 1167.6 |
| MA-PEG4- na-EDA- PNU159682 | 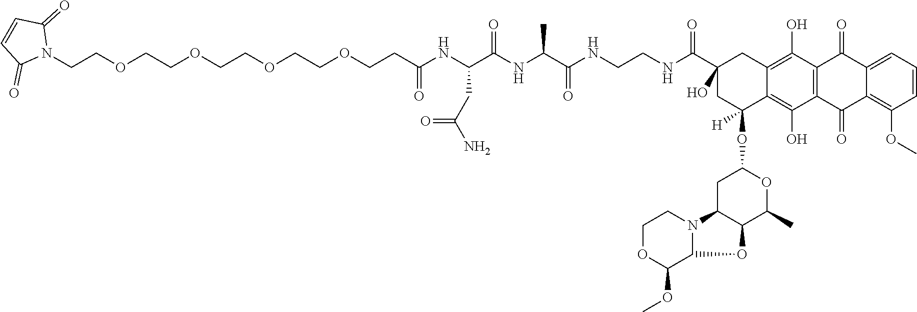 | 1182.2 | 1182.4 |
| MA-PEG4- EDA- PNU159682 | 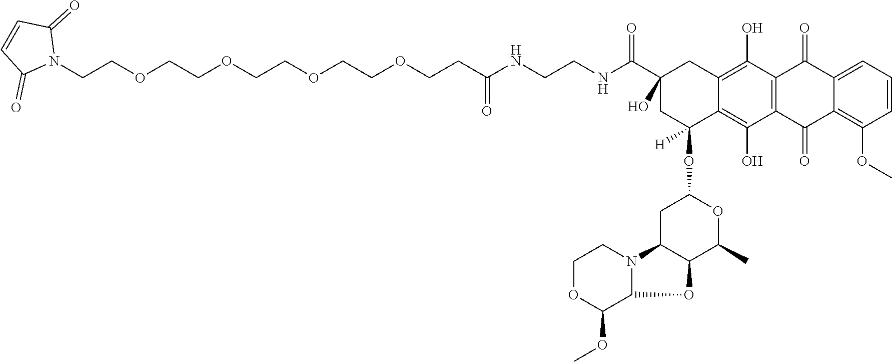 | 997.01 | 996.8 |
| MA-PEG4- vc-PAB- DMAE- PNU159682 | 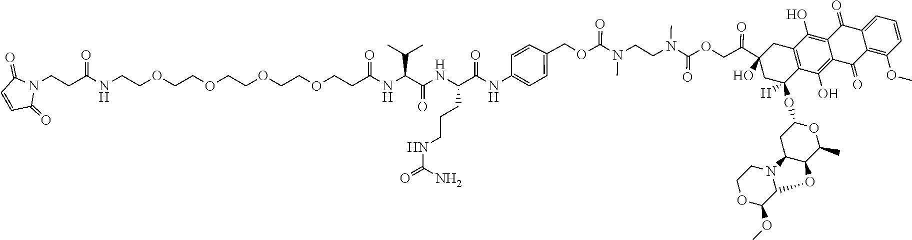 | 1559.6 | 1558.7 |

The present invention will be further understood by reference to the following examples.

### EXAMPLES

### Example 1—Generation of the Anti-ROR1 VNAR B1 Loop Library Sequences

### B1 Protein Library Design.

To gain a better understanding of the interaction between 1B1 and ROR1, we solved the crystal structure of 1B1 in complex with the ROR1 Ig domain (data not shown). This crystal structure informed which positions to change in the protein library that was expressed and screened. We had previously noted that mutation of B1 Tryptophan residues at positions 88 and 94 to Alanine (standard alanine scanning approach) caused loss of function or expression of the protein. From the crystal structure it was observed that these residues in the CDR3 loop appear to be important for ROR1 binding. A B1 loop library was therefore designed to modify the biophysical properties of the protein through changing selected positions within the CDR1 and CDR3 regions. The set of mutations made at each particular loop position was informed from the structural analysis of the B1:ROR1 complex, with a view to changing the biophysical properties whilst maintaining structural integrity and high affinity binding.

### Library Construction

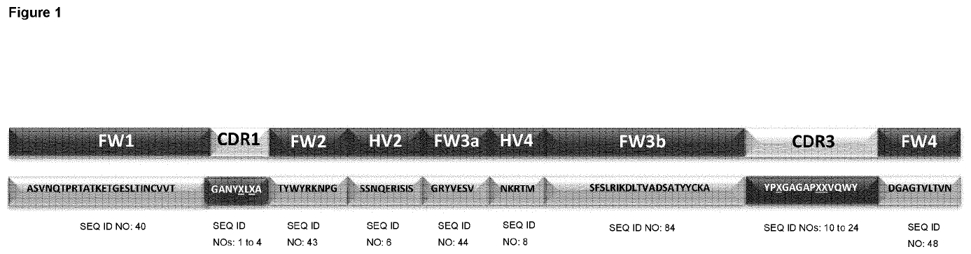

Sequence and loop library design of B1 are shown in FIG. 1. Library was synthesised by controlled mutagenesis of CDR1 and CDR3. Residues 30, 32, 88, 94 and 95 located within CDR loops were randomised.

### Libraries Construction.

B1 loop library DNA was amplified by PCR using specific primers to introduce Sfil restriction sites for cloning into pEDV1 phagemid vector. Library DNA ligated into pEDV1 was transformed into electrocompetent TG1 E. coli (Lucigen). The library size was calculated to be 8×104.

84 single clones were picked and sequenced as a quality control of the library. One sequence has been found to be WT B1 clone. In total 70 unique clones based on CDR1 and CDR3 diversity were identified. These sequences contain a C-terminal HisMyc tag to enable purification by IMAC chromatography and assessment of ROR1 binding by ELISA and flow cytometry

### Screening of the Library for ROR1 Specific VNAR Sequences

As B1 binds to both human and mouse ROR1, recombinant human and mouse ROR1-Fc protein was used for screening CDR loop library. In total 928 clones were expressed in 96 well format; periplasmic fractions were extracted and binding to ROR1 analysed in ELISA. 23 unique sequences additional to B1 WT sequence which binds to ROR1 were found.

### Expression of ROR1 VNAR Binders

23 clones were expressed in TG1 E. coli bacteria and IMAC purified using Ni-NTA Sepharose. Proteins were dialysed to PBS pH 7.4, absorbance Abs280 was measured and concentrations calculated. Yields obtained were in a range of 1.5 and 9 mg/L. Purity of proteins was analysed by SDS-PAGE.

Binding to human and mouse ROR1 by ELISA for the different loop variants is summarised in Table 5.

### Methods

### Library Synthesis

CDR loops library was synthetized by GeneArt Gene Synthesis according with provided design.

Library Subcloning into pEDV1

PCR amplification of 11.4 ng (10 μl) of synthetised library was performed in the total reaction volume 1 ml using Phusion High-Fidelity PCR Master Mix and the following primers:

280:
(SEQ ID NO: 133)
5′-CTACCGTGGCCCAGGCGGCC-3′
287:
(SEQ ID NO: 134)
5′-GGTGATGGTGGGCCCCTGAGGCCT-3′

Amplicons were purified with Promega PCR purification Kit, digested with Sfil and ligated into pEDV1 vector opened with Sfil restriction enzyme as well. Ligation performed at ratio 1:3 (0.54 μg vector ligated with 1.62 μg library DNA).

### Screening of CDR Loop Library: Periplasmic Expression of Single Clones in 96 Well Format and Binding ELISA

1. Inoculate Greiner 96 deep-well plate containing 1 ml 2×TY/0.1% glucose/100 μg/μl Amp. Grow for 5 h at 37° C., 180 rpm in incubation chamber until faintly turbid.
2. Induced with 110 μl/well 1 mM IPTG in 2×TY/Amp (final concentration of IPTG=100 μM); same shaking speed at 28° C. overnight.
3. Spin cultures for 15 min at 4° C. and 3500 rpm. Decant supernatant and tap dry on paper towels.
4. Add 250 μl/well ice-cold TES buffer (50 mM Tris/HCl, pH8.0/1 mM EDTA, pH8.0/20% Sucrose) to the pellets. Vortex.
5. Add 250 μl 1:5 diluted in water TES buffer (ice-cold). Keep on ice (or in the ridge) for 30 min.

Spin as above. Keep supernatants on ice until ready to use.

### ELISA

1. Coat 96 well plates with 1 μg/ml of huROR1-Fc, mouse ROR1-Fc, human ROR2-Fc or HSA and incubated overnight at 4° C.
2. Wash plates 3×PBST.
3. Block coated plates with 200 μl/well 4% MPBS. Incubate for 1 h at room temperature.
4. Wash 3×PBST.
5. Incubate plates with 100 μl/well of peri-prep for 1 h at room temperature.
6. Add 100 μl of anti-His-HRP (1:1000 in PBST) and incubate 1 h at room temperature.
7. Wash 2×PBST and 2× PBS.
8. Add 100 μl/well of TMB substrate. Stop reaction with 50 μl/well 1M H₂SO₄.

### Large Scale Expression and Purification of ROR1 VNAR Binders

1. Inoculate clones from glycerol stock into 20 ml of 2×TY/0.1% glucose/100 μg/μl Amp. Grow overnight at 37° C. shaking at 250 rpm in incubation chamber.
2. Dilute the overnight culture 1:50 in TB+phosphate salt+1% glucose+100 μg/ml Amp media (10 ml o/n culture into 500 ml media; 450 ml TB+50 ml phosphate salt) and incubate at 37° C. with vigorous shaking (250 rpm) all day or as long as possible.
3. Pellet the cells by centrifugation at 4,000×g for 15 min at 20° C.
4. Re-suspend the cells in the same volume of TB+phosphate salt+1% glucose+100 μg/ml Amp media and incubate at 30° C. overnight with shaking.
5. Pellet the cells by centrifugation at 4,000×g for 15 min at 20° C. and re-suspend the cells in the same volume of TB+phosphate salt+100 μg/ml Amp media (NO GLUCOSE). Add IPTG to a final conc. of 1 mM IPTG. Incubate at 30° C. for 4-5 h with shaking.
6. Collect the cells by centrifugation at 4500×g for 20 min [the pellet could be frozen at this point at −20° C.]
7. Re-suspend the pellet in 10% culture volume ice-cold TES buffer (50 ml for 500 ml culture) and shake gently on ice for 15 min.
8. Add an equal volume ice-cold 5 mM MgSO4 (for 2.5 mM final concentration of MgSO₄) and continue shaking gently on ice for a further 15 min.
9. Pellet the suspension by centrifugation at 8000×g for 30 min at 4° C. Supernatant contains released periplasmic proteins.
10. Add 10×PBS (pH 7.4) [final conc. of 1×PBS] to peri-prep extract prior to IMAC purification.

### Immobilised Metal Affinity Chromatography (IMAC) Purification

1. Add 2-3 ml Nickel-resin (His Pur Ni-NTA Resin, Thermo Fisher #88222) to 100 ml osmotic shock solution (periplasmic extract) and incubated on roller for 1 hour at room temperature.
2. Allow periplasmic extract to pass through the column (Poly prep chromatography columns 10 ml, Bio-Rad #7321010)
3. Wash the resin with 50-100 ml PBS.
4. Eluted protein with 5×1 ml 500 mM imidazole (pH 8).
5. Dialyze eluates in 3×5 liters PBS with agitation in dialysis cassette (Slide A Lyzer Dialysis cassette 7.000 MWCO, Thermo Fisher #66707)
6. Analyse proteins by SDS-PAGE.

Concentrations of purified proteins were determined from absorbance at 280 nm using the theoretical extinction coefficient predicted from the amino acid sequence. All proteins were characterised by reducing and non-reducing SDS PAGE analysis and mass spectrometry. The formation of the desired disulphide bond was confirmed by mass spectrometry methods.

### Size Exclusion Chromatography

Loop engineered variants were assessed by size exclusion chromatography. The monomericity and biophysical properties of B1 loop variants were assessed by size-exclusion chromatography (SEC) using an analytical SEC column (Superdex 75 10/300 GL). Chromatography was carried out in PBS pH 7.4. The elution volume on SEC can be a measure of the relative hydrophobicity of the different proteins. With increased elution volume, as a result of interactions with the column matrix, an indication of

increasing hydrophobicity.

The SEC elution volumes run under identical conditions are shown in Table 5.

### Binding to Human and Mouse ROR1 by BLI

Binding kinetics were determined using the Biolayer Interferometry (BLI) Octet K2 system (ForteBio). Human or mouse ROR1-hFc fusion proteins (extracelluar domains) were immobilised in sodium acetate pH5 buffer to COOH₂ chips or AR2G sensors using amine coupling. VNARs were tested at various concentrations and the Ka (M⁻¹s⁻¹), Kd (s⁻¹) and K_{D} (nM) values were determined using Octet Data Analysis High Throughput software (ForteBio) for Biolayer Interferometry.

Table 5. summarise the BLI data for the affinity of these molecules for human and mouse ROR1.

TABLE 5
Characterisation of B1 loop library variants

| | SEC | hROR1 | | | mROR1 | | | | |
|---|---|---|---|---|---|---|---|---|---|
| | RT | K_{D} | Kₐ | K_{d} | K_{D} | Kₐ | K_{d} | ELISA | |
| Name | (ml) | (nM) | (M⁻¹s⁻¹) | (s⁻¹) | (nM) | (M⁻¹s⁻¹) | (s⁻¹) | hROR1 | mROR1 |
| B1 | 27.79 | 2.1 | 120000 | 0.000252 | | | | | |
| 1E2 | 17.74 | 8.11 | 162600 | 0.001319 | | | | ** | — |
| 1E5 | 29.1 | 1.02 | 40200 | 0.0000411 | | | | *** | *** |
| 1B11 | 18.76 | 12.3 | 84500 | 0.00104 | 4.74E−09 | 2.35E+05 | 1.11E−03 | *** | * |
| C3CP | 18.65 | 4.56 | 165000 | 0.000753 | 3.21E−08 | 1.70E+05 | 5.44E−03 | *** | * |
| 2G5 | 18.16 | 9.49 | 452000 | 0.00429 | | | | ** | — |
| 1G12 | 17.72 | 39.9 | 225000 | 0.00897 | | | | ** | — |
| G5CP | 18.58 | 8.56 | 122000 | 0.00104 | 1.00E−07 | 1.00E+04 | 1.00E−03 | ** | — |
| 2F4 | 18 | 2.58 | 374000 | 0.000966 | | | | * | — |
| 1G9 | 18.52 | 13.4 | 112000 | 0.00151 | 1.78E−08 | 2.23E+05 | 3.96E−03 | *** | ** |
| 1H8 | 18.47 | 12.6 | 41300 | 0.000519 | 4.58E−09 | 1.82E+05 | 8.35E−04 | *** | ***/** |
| G11CP | 25.3 | 0.592 | 87900 | 0.0000521 | | | | ** | * |
| D9CP | 21.99 | 13.4 | 57200 | 0.000766 | | | | *** | * |
| 1B6 | 25.24 | 0.262 | 506000 | 0.000133 | | | | *** | *** |
| 1F10 | 25.65 | 0.602 | 147300 | 0.0000886 | | | | *** | *** |
| E6CP | 23.45 | 2.05 | 66500 | 0.000136 | | | | ** | — |
| F2CP | 20.6 | 9.43 | 48500 | 0.000458 | | | | *** | ** |
| B6CP | 20.76 | 2.1 | 79900 | 0.000168 | 5.13E−09 | 1.10E+05 | 5.63E−04 | *** | ** |
| 1G1 | 26.67 | 0.662 | 127400 | 0.0000844 | | | | *** | *** |
| A10CP | 18.07 | 11.6 | 157000 | 0.00182 | | | | * | — |
| G3CP | 20.58 | 0.796 | 369000 | 0.000294 | 2.80E−09 | 2.23E+05 | 6.23E−04 | *** | *** |

### Binding of Loop Variant VNARs to Cell-Surface ROR1 by Flow Cytometry

Loop variant VNARs were re expressed using intein technology. For expression as intein fusions, DNA encoding VNARs was optimised for E. coli expression (GeneArt, Thermo) and cloned in frame into an intein expression vector. This results in a gene encoding the VNAR protein of interest fused to an engineered intein domain which in turn is fused to a chitin binding domain (CBD) to enable purification on a chitin column.

Transformed E. coli cells were grown in 1 L shaker flasks until ODeoonm=˜0.6, cold shocked 4° C. for 2 hours then protein expression induced with 0.5 mM IPTG at 18° C. overnight. Cells were lysed by sonication in lysis buffer (50 mM sodium phosphate pH7.4, 0.5M NaCl, 15% glycerol, 0.5 mM EDTA, 0.1% Sarkosyl, 1 mM AEBSF) and centrifuged to remove cell debris. VNAR intein fusion protein was purified from clarified cell lysate by immobilising on chitin beads (NEB, S6651). Beads were washed extensively with lysis buffer followed by cleavage buffer (50 mM sodium phosphate pH6.9, 200 mM NaCl) and VNARs released from the beads by overnight chemical cleavage in 400 mM dioxyamine, or 0,0′-1,3-propanediylbishydroxylamine, or 100 mM cysteine or cysteamine to generate the corresponding C-terminal aminoxy, C-terminal cysteine or C-terminal thiol derivative of the VNARs.

Cleaved VNAR supernatant was then further purified by SEC (Superdex75 26/60 GE healthcare) and/or IMAC (HisTrap HP, GE Healthcare). Concentrations were determined from absorbance at 280 nm using the theoretical extinction coefficient predicted from the amino acid sequence. All proteins were characterised by reducing and non-reducing SDS PAGE analysis and mass spectrometry. The formation of the desired disulphide bond in the VNAR domain was confirmed by mass spectrometry methods. These C-terminal HisMyc tagged proteins were then assessed for ROR1 cell-surface binding by flow cytometry

Cell surface binding of test agents to hROR1 was characterized in different cell lines (A549 and A427) and the resulting Kugp, values determined. Adherent cancer cells were detached from tissue culture flasks by incubating with 0.1% EDTA/PBS solution at 37° C. for ˜10 minutes or until cells detached easily. Cells were re-suspended in ice-cold PBS/2% FCS in 15 ml tubes and centrifuged at 1500 rpm for 5 mins at 4° C. Supernatant was removed and the cell pellet re-suspended in PBS/2% FCS. A cell count was performed using a Z1 Coulter Particle Counter (Beckman Coulter) or Chemometec Nucleocounter NC-202 and 5×10≡cells were aliquoted per test sample into a 96 well plate. Cells were incubated with 100 μl of test agents at a range of concentrations, plus controls for 1 hr on ice. The sample plate was centrifuged at 2000 rpm for 5 mins. The supernatant was removed and a wash performed by re-suspending the cell pellets in 0.25 mL of ice-cold PBS/2% FCS using a multichannel pipette. Samples were again centrifuged at 2000 rpm for 5 min at 4° C. Supernatant was removed and two further washes performed as described. After the final wash and centrifugation step, excess liquid was removed by blotting the plate on tissue paper. Binding of VNARs was determined by adding 100 μl of anti-x6His tag Ab (Abcam) per cell pellet sample as appropriate and incubated on ice for 30 mins. Wash steps were performed as described previously. PE-anti-mouse antibody (JIR) was used to detect binding of the VNAR (His₆ tagged) agents and corresponding drug-conjugates by incubating with the appropriate samples for 30 min on ice in the dark. Wash steps were performed as described previously. All cell pellets were finally re-suspended in 0.3 ml of ice-cold PBS/2% FCS and left on ice in the dark prior to analysis on a Merck-Millipore Guava EasyCyte HT or Thermo Fisher Attune NxT flow cytometer.

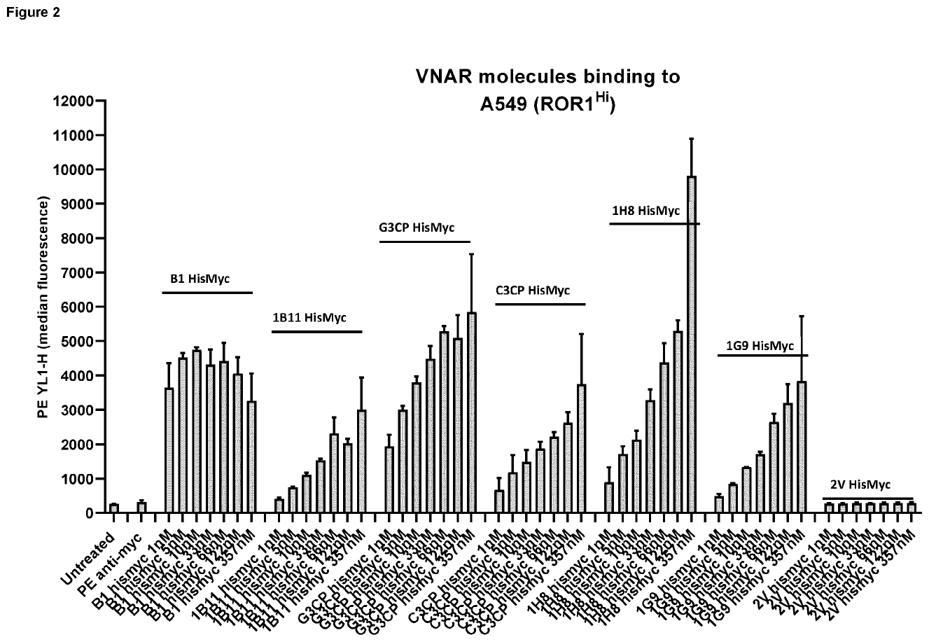

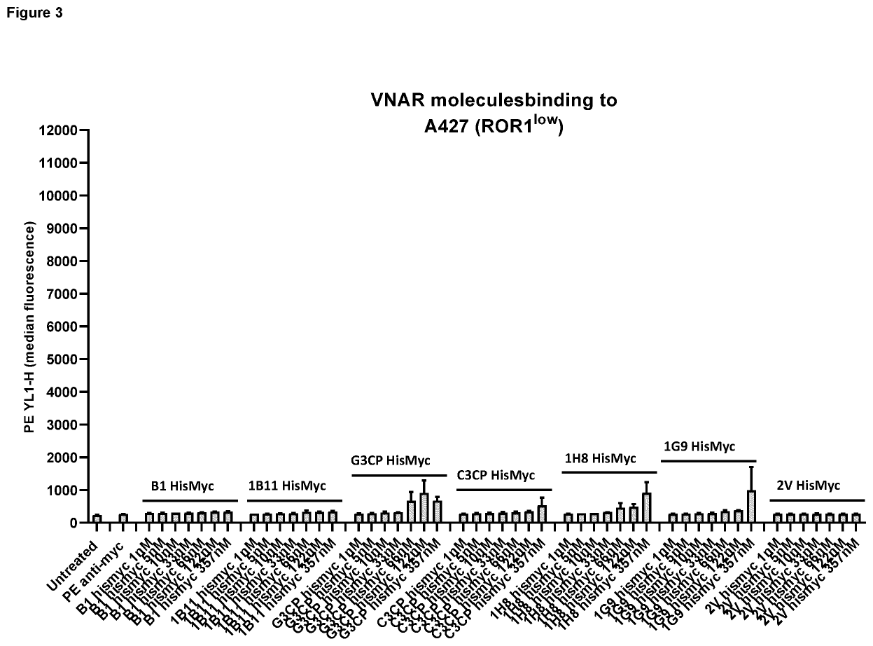

As shown in FIG. 2 and FIG. 3, the loop library variants bind to the ROR1^{hi} human cancer cell-line A549 but not to the ROR1^{low} human cancer cell-line A427, 2V is a control VNAR sequence, derived from a naïve VNAR library, so is representative of this protein class but has no known target.

### Example 2—Humanisation and Further Engineering of B1 Loop Library Variants

Humanised sequence derivatives of three lead ROR1 binding B1 loop library VNARs were generated using two different strategies.

Humanised sequences were designed based on the human germ line VK1 sequence, DPK-9. For example, in P3A1 V1 the framework regions 1, 3 and 4 of the VNAR were mutated to align with the framework regions of DPK-9.

The second strategy involved grafting the binding loops of the ROR1 binding VNARs onto a previously humanised VNAR framework (Kovalenko et al JBC 2013 288(24) 17408-17419; WO2013/167883). But with further positions engineered based on the structure of the VNAR B1 in complex with the ROR1 Ig domain.

Additional sites of engineering include amino acid changes in the CDR1, HV2 and HV4 regions of the protein.

Similarly, a humanised variant of B1 was developed using this approach, which accordingly contains amino acid changes in its CDR1, HV2 and HV4 regions as well as the framework regions. So B1G4 is, by de facto, a loop library derivative of B1 or a loop library variant of humanised variants of B1 whereby the CDR1, HV2, HV4 and CDR3 sequences are the same as in the parental protein.

Examples of humanised/grafted loop library VNAR sequences are below:

G3CP G4
(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK
G3CP V15
(SEQ ID NO: 72)
ASVTQSPRSASKETGESLTITCRVTGANYGLAATYWYRKNPGSSNQERIS
ISGRYSESVNKRTMSFSLRISSLTVEDSATYYCKAYPWGAGAPYNVQWYD
GQGTKLEVK
1H8 G4
(SEQ ID NO: 73)
TRVDQSPSSLSASVGDRVTITCVLTDANYGLAATYWYRKNPGSSNKERIS
ISGRYSESVNKGTMSFTLTISSLQPEDSATYYCRAYPWGAGAPSSVQWYD
GAGTKVEIK
1H8 V15
(SEQ ID NO: 74)
ASVTQSPRSASKETGESLTITCRVTGANYGLAATYWYRKNPGSSNQERIS
ISGRYSESVNKRTMSFSLRISSLTVEDSATYYCKAYPWGAGAPSSVQWYD
GQGTKLEVK
C3CP G4
(SEQ ID NO: 75)
TRVDQSPSSLSASVGDRVTITCVLTDANYGLAATYWYRKNPGSSNKERIS
ISGRYSESVNKGTMSFTLTISSLQPEDSATYYCRAYPWGAGAPRQVQWYD
GAGTKVEIK
C3CPV15
(SEQ ID NO: 76)
ASVTQSPRSASKETGESLTITCRVTGANYGLAATYWYRKNPGSSNQERIS
ISGRYSESVNKRTMSFSLRISSLTVEDSATYYCKAYPWGAGAPRQVQWYD
GQGTKLEVK
B1G4
(SEQ ID NO: 51)
TRVDQSPSSLSASVGDRVTITCVLTDANYGLAATYWYRKNPGSSNKERIS
ISGRYSESVNKGTMSFTLTISSLQPEDSATYYCRAYPWGAGAPWLVQWYD
GAGTKVEIK.

DNA encoding the humanised constructs was codon optimised for expression in E. coli and synthesised by GeneArt (Thermo). All humanised sequences were generated with the following C terminal His₆ tag: QASGAHHHHHH (SEQ ID NO: 102)

G4 sequences were made without an additional C-terminal tag.

DNA encoding these proteins was sub cloned into the intein expression vectors, expressed in E. coli and purified as described previously in “Typical method for expression of VNAR intein fusion proteins” section.

Humanised ROR1 binding VNAR variants demonstrated high affinity binding to human ROR1 by BLI, good thermal stability and little evidence of aggregation by SEC. BLI was performed as described previously using human ROR1 ECD—Fc immobilised to the chip surface. SEC was performed as previously described. Thermal stability assays used Applied Biosystems StepOne Real Time PCR system with the Protein Thermal Shift™ dye kit (Thermo). The assay mix was set up so that the protein was at a final concentration of 20 μM in 20 μL. 5 μL of Thermal Shift™ buffer was added alongside 2.5 uL 8× Thermal Shift™ Dye. Assays were run using the StepOne software and data analysed using Protein Thermal Shift™ software. All data are from first derivative analysis. BLI data for hROR1 binding and thermal stability by protein thermal shift is shown in Table 6.

TABLE 6
Thermal stability and hROR1 binding
data for humanised VNAR loop variants

| | Tm | hROR1 binding |
|---|---|---|
| Construct | (° C.) | K_{D} (nM) |
| G3CP G4 | 57.4 | 8.5 |
| G3CP V15 | 57.2 | <0.5 |
| 1H8 G4 | 58.7 | 33.7 |
| 1H8 V15 | 57.4 | <0.5 |
| C3CP G4 | 58.2 | 6.8 |
| C3CP V15 | 57.2 | 1.4 |
| B1G4 | 56.0 | 9.2 |

Either grafting the HV and CDR loops of G3CP, 1 H₈ and C₃CP onto a humanised VNAR framework coupled with additional mutations in the CDR1, HV2 and HV4 regions or substituting VNAR framework sequences with regions from the human DPK-9 sequence, yielded substantially engineered proteins that are stable, monomeric and maintain high affinity binding to hROR1.

### Example 3—Generation of the Anti-ROR1 VNAR P3A1 G1 Loop Library Sequences

### Library Design

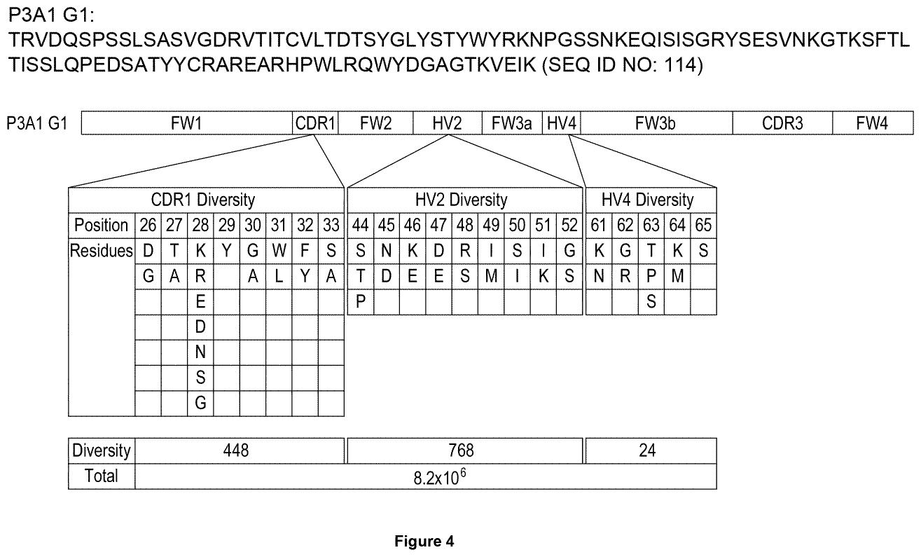

P3A1 G1 is a humanised version of the ROR1 binding VNAR P3A1. The P3A1 G1 loop library was designed to improve ROR1 binding affinity of this humanised variant via randomisation of CDR1, HV2 and HV4 regions without any changes within frameworks. Choice of mutations was made based on the data analysis of VNAR sequences from Squalus acanthus. Sequence of P3A1 G1 and library design are shown in FIG. 4.

Library was synthesised by controlled mutagenesis of CDR1, HV2 and HV4. Residues 26-33, 44-52 and 61-65 located within CDR1, HV2 and HV4 loops respectively were changed to selected amino acids as specified in FIG. 4 resulting in total library diversity of 8.2×10⁶ combinations.

### Libraries Construction.

P3A1 G1 library DNA was amplified by PCR using specific primers to introduce Sfil restriction sites for cloning into pEDV1 phagemid vector. This introduces an additional Ser residue into CD1. Library DNA ligated into pEDV1 was transformed into electrocompetent TG1 E. coli (Lucigen). The library size was calculated to be 2×10⁸. 192 single clones were picked and sequenced as a quality control of the library.

### Screening of P3A1 G1 Library for Antigen Specific VNAR Sequences.

Recombinant human ROR1 protein was used for selections and screening of the P3A1 G1 library. Two strategies were utilised to isolate ROR1 specific binders: selections on biotinylated antigen immobilised on pre-decorated streptavidin-coated beads and selections with antigen directly immobilised to the immunotube. Selection on pre-decorated with biotinylated antigen beads involved 3 rounds of panning with low stringency in first and second rounds (3×PBST and 3×PBS washes for both rounds, 100 nM and 10 nM of biotinylated huROR1 for round 1 and 2 respectively), but high stringency for third round (10×PBST and 10×PBS washes, 0.5 nM of biotinylated huROR1). Selection on immunotubes consists of 2 rounds of panning with constant antigen concentration of 2 ng/ml. Following the selection process, outputs were screened for antigen-specific binding by monoclonal phage and periplasmic extract ELISAs against human or mouse ROR1. 95% of monoclonal phage displaying the VNARs were specific to human and mouse ROR1 from selections with antigen directly immobilised to the immunotube and 4% for selections on biotinylated antigen immobilised on pre-decorated streptavidin-coated beads. In total 9 unique sequences from each selection campaign were expressed and analysed for binding and selectivity (Table 7 and 8).

TABLE 7
G1 loop variants isolated from selections with antigen directly immobilised to the immunotube.

| Clone | |
|---|---|
| ID | Sequence-including C-terminal tag: QASGAHHHHHHGAEFEQKLISEEDL (SEQ ID NO: 98) |
| P3A1 | TRVDQTPSSLSASVGDRVTITCVLTDTSYGLYSTYWYRKNPGSSNKEQISISGRYSESVNKGTKSFTLTISSLQPEDSATYY |
| G1 | CRAREARHPWLRQWYDGAGTKVEIKQASGAHHHHHHGAEFEQKLISEEDL (SEQ ID NO: 135) |
| NAG8.S | TRVDQSPSSLSASVGDRVTITCVLTGTKYGLYASTYWYRKNPGSPNKDRMIIGGRYSESVNNGTKSFTLTISSLQPEDSATYY |
| | CRAREARHPWLRQWYDGAGTKVEIKQASGAHHHHHHGAEFEQKLISEEDL (SEQ ID NO: 136) |
| AC5.S | TRVDQSPSSLSASVGDRVTITCVLTDTSYGLYSSTYWYRKNPGSSNKERMSISGRYSESVNKGSKSFTLTISSLQPEDSATYY |
| | CRAREARHPWLRQWYDGAGTKVEIKQASGAHHHHHHGAEFEQKLISEEDL (SEQ ID NO: 137) |
| AG5.S | TRVDQSPSSLSASVGDRVTITCVLTDTNYALYSSTYWYRKNPGSTNKESMSIGGRYSESVNKRSKSFTLTISSLQPEDSATYY |
| | CRAREARHPWLRQWYDGAGTKVEIKQASGAHHHHHHGAEFEQKLISEEDL (SEQ ID NO: 138) |
| AF1.S | TRVDQSPSSLSASVGDRVTITCVLTDAKYGLYSSTYWYRKNPGSPDKERIINGGRYSESVNNRTKSFTLTISSLQPEDSATYY |
| | CRAREARHPWLRQWYDGAGTKVEIKQASGAHHHHHHGAEFEQKLISEEDL (SEQ ID NO: 139) |
| AB11.S | TRVDQSPSSLSASVGDRVTITCVLTDARYGLFASTYWYRKNPGSPDKDSISIGGRYSESVNKRSKSFTLTISSLQPEDSATYY |
| | CRAREARHPWLRQWYDGAGTKVEIKQASGAHHHHHHGAEFEQKLISEEDL (SEQ ID NO: 140) |
| NAE1.S | TRVDQSPSSLSASVGDRVTITCVLTGTKYGLFSSTYWYRKNPGSSDKERISIGGRYSESVNKGPKSFTLTISSLQPEDSATYY |
| | CRAREARHPWLRQWYDGAGTKVEIKQASGAHHHHHHGAEFEQKLISEEDL (SEQ ID NO: 141) |
| AA6.S | TRVDQSPSSLSASVGDRVTITCVLTGTRYGLYASTYWYRKNPGSPDEESMINGGRYSESVNKRTKSFTLTISSLQPEDSATYY |
| | CRAREARHPWLRQWYDGAGTKVEIKQASGAHHHHHHGAEFEQKLISEEDL (SEQ ID NO: 142) |
| AB7.S | TRVDQSPSSLSASVGDRVTITCVLTDTKYALFSSTYWYRKNPGSSNEERISIGGRYSESVNNRTKSFTLTISSLQPEDSATYY |
| | CRAREARHPWLRQWYDGAGTKVEIKQASGAHHHHHHGAEFEQKLISEEDL (SEQ ID NO: 143) |
| AF7.S | TRVDQSPSSLSASVGDRVTITCVLTGTKYGLYASTYWYRKNPGSTDKERIIIGGRYSESVNNRSKSFTLTISSLQPEDSATYY |
| | CRAREARHPWLRQWYDGAGTKVEIKQASGAHHHHHHGAEFEQKLISEEDL (SEQ ID NO: 144) |

TABLE 8
P3A1 G1 loop variants isolated from selections on biotinylated
antigen immobilised on pre-decorated streptavidin-coated beads.

| Clone | |
|---|---|
| ID | Sequence-including C-terminal tag: QASGAHHHHHHGAEFEQKLISEEDL (SEQ ID NO: 98) |
| P3A1 | TRVDQTPSSLSASVGDRVTITCVLTDTSYGLYSTYWYRKNPGSSNKEQISISGRYSESVNKGTKSFTLTISSLQPEDSATYYCR |
| G1 | AREARHPWLRQWYDGAGTKVEIKQASGAHHHHHHGAEFEQKLISEEDL (SEQ ID NO: 135) |
| NAC1.S | TRVDQSPSSLSASVGDRVTITCVLTDTKYGLYSSTYWYRKNPGSTDEERIIIGGRYSESVNKGSKSFTLTISSLQPEDSATYYC |
| | RAREARHPWLRQWYDGAGTKVEIKQASGAHHHHHHGAEFEQKLISEEDL (SEQ ID NO: 168) |
| NAC6.S | TRVDQSPSSLSASVGDRVTITCVLTDTRYALYSSTYWYRKNPGSTDEERISIGGRYSESVNKGSKSFTLTISSLQPEDSATYYC |
| | RAREARHPWLRQWYDGAGTKVEIKQASGAHHHHHHGAEFEQKLISEEDL (SEQ ID NO: 169) |
| NAE9.S | TRVDQSPSSLSASVGDRVTITCVLTGTGYGLYSSTYWYRKNPGSTDEERMIISGRYSESVNKRTKSFTLTISSLQPEDSATYYC |
| | RAREARHPWLRQWYDGAGTKVEIKQASGAHHHHHHGAEFEQKLISEEDL (SEQ ID NO: 170) |
| NAF3.S | TRVDQSPSSLSASVGDRVTITCVLTDTKYGLYASTYWYRKNPGSPDEERIIISGRYSESVNKGSKSFTLTISSLQPEDSATYYC |
| | RAREARHPWLRQWYDGAGTKVEIKQASGAHHHHHHGAEFEQKLISEEDL (SEQ ID NO: 171) |
| NAF5.S | TRVDQSPSSLSASVGDRVTITCVLTDTKYALFSSTYWYRKNPGSTNEDRISIGGRYSESVNKGTKSFTLTISSLQPEDSATYYC |
| | RAREARHPWLRQWYDGAGTKVEIKQASGAHHHHHHGAEFEQKLISEEDL (SEQ ID NO: 172) |
| AA5.S | TRVDQSPSSLSASVGDRVTITCVLTGTGYGLYSSTYWYRKNPGSSDEERIIIGGRYSESVNKGSKSFTLTISSLQPEDSATYYC |
| | RAREARHPWLRQWYDGAGTKVEIKQASGAHHHHHHGAEFEQKLISEEDL (SEQ ID NO: 173) |
| AD10.S | TRVDQSPSSLSASVGDRVTITCVLTGTKYGLYSSTYWYRKNPGSSDKESIIIGGRYSESVNNGTKSFTLTISSLQPEDSATYYC |
| | RAREARHPWLRQWYDGAGTKVEIKQASGAHHHHHHGAEFEQKLISEEDL (SEQ ID NO: 174) |
| AE3.S | TRVDQSPSSLSASVGDRVTITCVLTGTRYGLYSSTYWYRKNPGSSDEERISISGRYSESVNKGTKSFTLTISSLQPEDSATYYC |
| | RAREARHPWLRQWYDGAGTKVEIKQASGAHHHHHHGAEFEQKLISEEDL (SEQ ID NO: 175) |
| AF4.S | TRVDQSPSSLSASVGDRVTITCVLTDTRYALYASTYWYRKNPGSTDEDRIIIGGRYSESVNKGSKSFTLTISSLQPEDSATYYC |
| | RAREARHPWLRQWYDGAGTKVEIKQASGAHHHHHHGAEFEQKLISEEDL (SEQ ID NO: 176) |

### Expression of ROR 1 P3A 1 G1 Loop Variants

Clones were expressed in TG1 E. coli bacteria and the resulting C-terminally HisMyc-tagged proteins were purified by IMAC using Ni-NTA Sepharose. Proteins were dialysed to PBS pH 7.4, absorbance Abs280 was measured and concentrations calculated. Yields obtained were in a range of 0.5 and 6.5 mg/L. Purity of proteins was analysed by SDS-PAGE.

All proteins were characterised by reducing and non-reducing SDS PAGE analysis and mass spectrometry. The formation of the desired disulphide bond was confirmed by mass spectrometry methods.

Binding of P3A 1 G1 Loop Variants to hROR 1 by ELISA

The binding of P3A1 G1 loop variants to human ROR1 was initially assessed by ELISA. In brief, ELISA method as follows. Wells coated with 100ng of ROR1-hFc antigen and incubated, covered, at room temperature for 2 hr. Plates washed 3×400ul per well with PBST (PBS+0.05% Tween 20 (v/v)), then blocked with 4% skimmed milk powder (w/v) in PBST for 1 hour at 37° C. Plates washed as before plus additional wash in PBS alone. HisMyc-tagged binding proteins were diluted in 4% milk PBST and incubated overnight at 4° C. Plates washed 3× with PBST, 3× PBS and binding detected using appropriate secondary detection antibody in 4% milk PBST, room temperature 1 hour. Secondary antibodies used include:
Anti-c-Myc, HRP (Invitrogen #R951-25)
Mouse anti-polyHis, HRP (Sigma #A7058)

Plates washed 3× with PBST. 1 OOpL TMB substrate (Thermo #34029) added and reaction allowed to proceed at r.t. for 10 mins. 100 μL of 2M H₂SO₄ added to quench the reaction. Plate centrifuged briefly before absorbance at 450 nm read on a CLARIOstar plate reader (BMG Labtech).

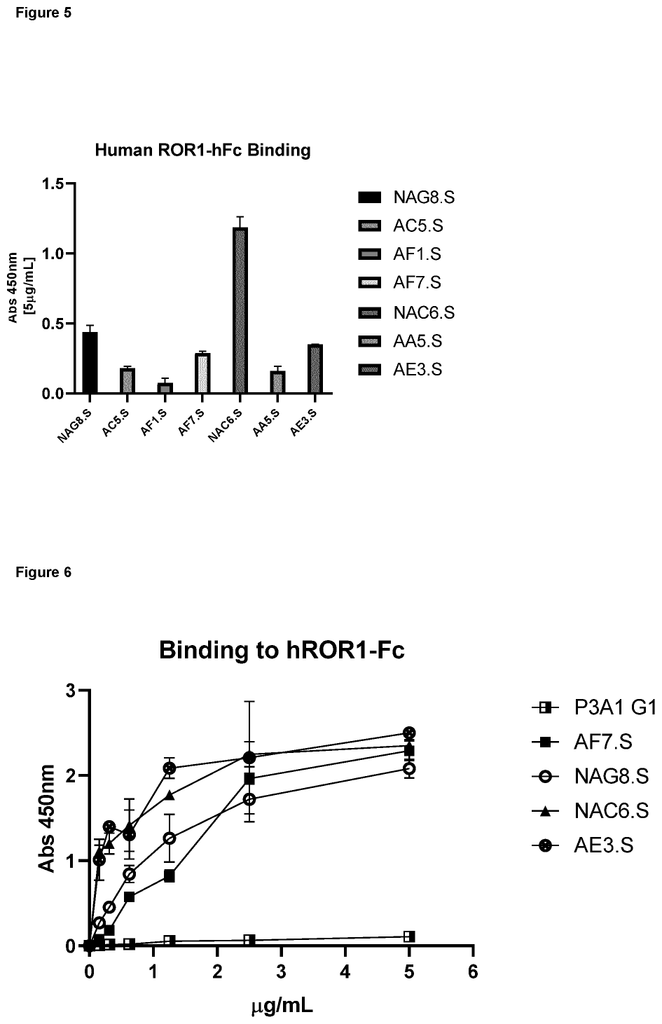

FIG. 5 shows the relative binding of different variants to human ROR1 with sequences NAG8.S, AF7.S, NAC6.S and AE3.S showing the strongest signal for binding.

The same ELISA method was also used to compare binding of variants NAG8.S, AF7.S, NAC6.S and AE3.S to human ROR1 with that of the parental P3A1 G1 sequence.

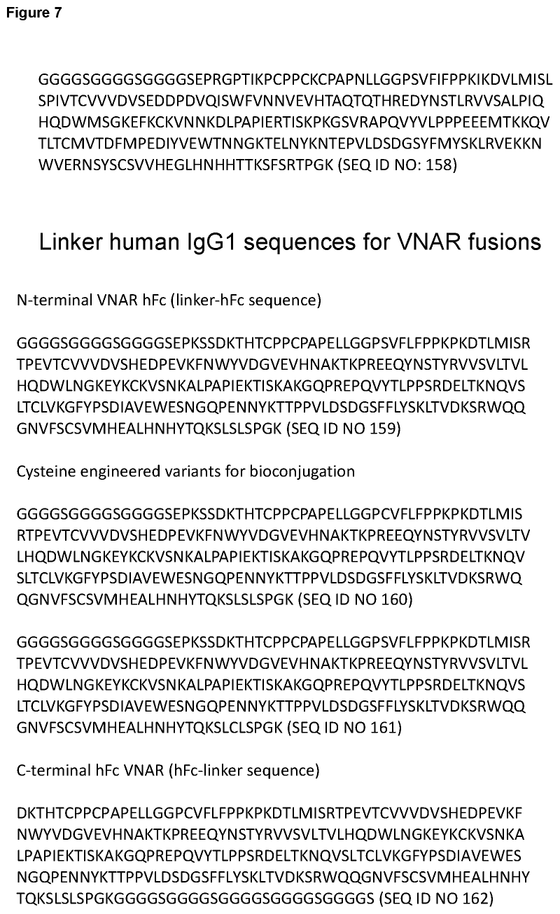

The dose response data shown in FIG. 6 shows that these loop library sequences bind stronger to human ROR1 than the parental P3A1 G1 protein.

### Characterisation of Mouse ROR1 and ROR2 Binding of P3A1 G1 Loop Variants by ELISA

A selection of P3A1 G1 loop variants were further characterised for binding to mouse ROR1 and human ROR2 by ELISA. The same ELISA procedure was employed as described above but with either mROR1-hFc or hROR2-hFc coated on the plates. None of the variants tested bound to human ROR2. Of the variants that were tested, NAC6.S and AE3.S bound to mouse ROR1

### Expression of P3A 1 G1 Loop Library Variants as Intein Fusion Proteins

P3A1 G1 loop variant VNARs NAG8.S, NAC6.S and AE3.S were re-expressed using intein technology but with a Ser deletion from the CDR1 loop. Expression as intein fusions was performed as described above with either a His tag QACKAHHHHHHG (SEQ ID NO: 163) or HisMyc tag QACKAHHHHHHGAEFEQKLISEEDLG (SEQ ID NO: 164) incorporated at the C-terminus of the VNAR domain.

Following expression and capture on chitin beads the intein VNARs were released from the beads by overnight chemical cleavage in 400 mM dioxyamine, or O,O′-1,3-propanediylbishydroxylamine, or 100 mM cysteine or cysteamine to generate the corresponding C-terminal aminoxy, C-terminal cysteine or C-terminal thiol derivative of the VNARs.

Cleaved VNAR supernatant was then further purified by SEC (Superdex75 26/60 GE healthcare) and/or IMAC (HisTrap HP, GE Healthcare) to give the proteins NAG8, NAC6 and AE3. Concentrations were determined from absorbance at 280 nm using the theoretical extinction coefficient predicted from the amino acid sequence. All proteins were characterised by reducing and non-reducing SDS PAGE analysis and mass spectrometry. The formation of the desired disulphide bond in the VNAR domain was confirmed by mass spectrometry methods.

These C-terminal HisMyc or His tagged proteins were then further assessed for ROR1 binding by BLI, thermal stability and biophysical properties by SEC,

### Binding to Human and Mouse ROR1 by BLI

Binding kinetics were determined using the Biolayer Interferometry (BLI) Octet K2 system (ForteBio). Human or mouse ROR1-hFc fusion proteins (extracelluar domains) were immobilised in sodium acetate pH5 buffer to COOH₂ chips or AR2G sensors using amine coupling. VNARs were tested at various concentrations and the Ka (M⁻¹s⁻¹), Kd (s⁻¹) and K_{D} (nM) values were determined using Octet Data Analysis High Throughput software (ForteBio) for Biolayer Interferometry. Binding parameters are shown in Table 9.

### Thermal Stability Assays

Thermal stability assays used Applied Biosystems StepOne Real Time PCR system with the Protein Thermal Shift™ dye kit (Thermo). The assay mix was set up so that the protein was at a final concentration of 20 μM in 20 μL in PBS pH 74. 2.5 μL 8× Thermal Shift™ Dye was added. Assays were run using the StepOne software and data analysed using Protein Thermal Shift™ software. All data are from first derivative analysis with the Tm values detailed in Table 9.

### Size Exclusion Chromatography

The monomericity and biophysical properties of P3A1 loop variants were assessed by size-exclusion chromatography (SEC) using an analytical SEC column (Superdex 75 increase 10/300 GL). Chromatography was carried out in PBS pH 7.4.

The % monomericity and SEC elution volumes run under identical conditions are shown in Table 9.

TABLE 9
Summary of ROR1 binding and physical properties
of P3A1 G1 variants NAG8, NAC6 and AE3

| | | P3A1 | P3A1 | P3A1 |
|---|---|---|---|---|
| | P3A1 | G1 AE3 | G1 NAC6 | G1 NAG8 |
| Parameter | G1 | His | His | HisMyc |
| hROR1 affinity K_{D} (nM) | Weak binder | 5 | 13.8 | 12.2 |
| hROR1 affinity kₒₙ (M⁻¹s⁻¹) | — | 1.32E+06 | 1.38E+06 | 1.48E+06 |
| hROR1 affinity k_{off} (s⁻¹) | — | 6.60E−03 | 1.90E−02 | 1.80E−02 |
| % monomer by SEC | 100 | 100 | 100 | 100 |
| SEC elution volume (mL) | 15.47 | 17.3 | 17.3 | 14.8 |
| Tm (° C.) | 58.01 | 63.2 | 58.9 | 53.8 |
| mouseROR1 K_{D} (nM) | — | 4.4 | 26.1 | 13.3 |

### Example 4—VNAR Reformatting as Multimers

ROR1 binding loop variant VNARs were successfully reformatted into hetero dimers and trimers by genetic fusion using different GlySer based linkers to generate bi-specific binders, ROR1 bi-paratopic binders and ROR1 bi-paratopic bi-specific binders.

### Bispecific Binders

Several bi-specific VNAR-based binders were developed by combining ROR1 loop-variant VNAR binders with the humanised VNAR BA11, which binds with high affinity to serum albumins, using a PGVQPSPGGGGGS (SEQ ID NO: 96) linker

Proteins were expressed with a C-terminal tag QACKAHHHHHHGAEFEQKLISEEDL (SEQ ID NO: 97) or QACKAHHHHHH (SEQ ID NO: 104) to aid purification and characterisation. This tag also contains a single cysteine residue to facilitate site-selective bioconjugation of payloads to the proteins using thiol mediated chemical coupling strategies

Binding kinetics were determined using Biolayer interferometry (K2 Octet instrument/Pall ForteBio) as previously described. For BLI experiments ROR1-hFc, (extracellular domain) and HSA were immobilised in sodium acetate pH5 buffer to AR2G sensors using amine coupling. VNAR-based molecules were tested at various concentrations and the Ka (M⁻¹s⁻¹), Kd (s⁻¹) and K_{D} (nM) values were determined using the Octet data analysis HT software (Pall ForteBio).

Binding kinetics for hROR1 binding were also performed with saturating levels of HSA (200 nM) in the baseline, association and dissociation conditions.

Binding to the ROR1 ^{hi} A549 cancer-cell lines was determined by flow cytometry. A dose response was performed and the K_{Dapp} for cell-surface ROR1 binder determined using the change in mean fluorescence intensity (background corrected) as a function of VNAR concentration.

The characterisation of these bi-specific VNARs is shown in Table 10.

TABLE 10
Characterisation of bi-specific proteins containing ROR1 VNAR loop library variants

| | BA11- | BA11- | G3CP- | BA11- | 1H8- | G3CP V15- | G3CP G4- |
|---|---|---|---|---|---|---|---|
| | B1 | G3CP | BA11 | 1H8 | BA11 | BA11 | BA11 |
| Expected Mass (Da) | 27121.0 | 27170.0 | 27170.0 | 27066.9 | 27066.9 | 27264.1 | 27160.9 |
| Observed Mass (Da) | 27116.5 | 27165.9 | 27165.7 | 27062.6 | 27062.4 | 27260.1 | 27156.8 |
| hROR1 affinity K_{D} (nM) | 0.21 | 2.61 | 1.74 | 6.83 | 4.74 | 1.47 | 10.2 |
| hROR1 affinity kₒₙ (M⁻¹s⁻¹) | 9.70E+04 | 1.04E+05 | 1.13E+05 | 7.02E+04 | 8.26E+04 | 9.00E+04 | 1.52E+05 |
| hROR1 affinity k_{off} (s⁻¹) | 2.03E−05 | 2.73E−04 | 1.96E−04 | 4.79E−04 | 3.92E−04 | 1.33E−04 | 1.55E−03 |
| HSA affinity K_{D} (nM) | 3.15 | 2.11 | 5.36 | 3.17 | 3.07 | 1.49 | 3.28 |
| HSA affinity kₒₙ (M⁻¹s⁻¹) | 8.48E+04 | 2.91E+05 | 1.17E+05 | 2.09E+05 | 2.57E+05 | 2.59E+05 | 2.15E+05 |
| HSA affinity k_{off} (s⁻¹) | 2.67E−04 | 6.12E−04 | 6.25E−04 | 6.63E−04 | 7.88E−04 | 3.85E−04 | 7.06E−04 |
| hROR1 affinity in presence | 3.05 | 26.7 | 6.21 | 50.8 | 29.4 | 11.0 | 23 |
| of HSA K_{D} (nM) | | | | | | | |
| hROR1 affinity in presence | 1.00E+05 | 4.87E+04 | 1.12E+05 | 4.59E+04 | 4.17E+04 | 6.20E+04 | 4.22E+03 |
| of HSA kₒₙ (M⁻¹s⁻¹) | | | | | | | |
| hROR1 affinity in presence | 3.06E−04 | 1.30E−03 | 6.95E−04 | 2.33E−03 | 1.22E−03 | 6.80E−04 | 1.15E−02 |
| of HSA k_{off} (s⁻¹) | | | | | | | |
| Binding to ROR1⁺ A549 | 5.42 nM | 2.22 nM | 1.74 nM | 2.61 nM | 6.61 nM | ND | 4.30 nM |
| cells [K_{D} app] | | | | | | | |

Bi-specific VNAR binders were further modified through conjugation to the single cysteine residue in the C-terminal tag.

### Bi-Paratopic Binders

Several ROR1 bi-paratopic binders were developed combining different ROR1 loop-variant VNAR binders with or without additional insertion of the serum albumin binding humanised VNAR BA11. The VNAR domains were joined together using a PGVQPAPGGGGS (SEQ ID NO: 90) linker and proteins were expressed with a C-terminal tag QACKAHHHHHHGAEFEQKLISEEDL (SEQ ID NO: 97) or QACKAHHHHHH (SEQ ID NO: 104) to aid purification and characterisation. This tag also contains a single cysteine residue to facilitate site-selective bioconjugation of payloads to the proteins using thiol mediated chemical coupling strategies

Binding kinetics were determined using Biolayer interferometry (K2 Octet instrument/Pall ForteBio) as previously described. For BLI experiments ROR1-hFc, (extracelluar domain) was immobilised in sodium acetate pH5 buffer to AR2G sensors using amine coupling. VNAR-based molecules were tested at various concentrations and the Ka (M⁻¹s⁻¹), Kd (s⁻¹) and K_{D} (nM) values were determined using the Octet data analysis HT software (Pall ForteBio).

The characterisation of these bi-specific VNARs is shown in Table 11.

TABLE 11
Characterisation of bi-paratopic proteins containing
ROR1 VNAR loop library variants

| | | Expected | | | | |
|---|---|---|---|---|---|---|
| | | Mass | Observed | hROR1 binding (BLI) | | |
| Construct | Linker | (reduced); Da | Mass; Da | K_{D} (nM) | Kₐ (M⁻¹s⁻¹) | Kd (s⁻¹) |
| P3A1G1AE3- | PGVQPAPGGGGS | 26288.0 | 26284.4 | 0.58 | 4.46E+05 | 2.58E−04 |
| G3CPG4 | (SEQ ID NO: 90) | | | | | |
| G3CPG4 | PGVQPAPGGGGS | 26288.0 | 26284.5 | 0.53 | 4.11E+05 | 2.17E−04 |
| P3A1G1AE3 | (SEQ ID NO: 90) | | | | | |
| P3A1-BA11-G3CP | PGVQPAPGGGGS | 40146.51 | ND | 0.22 | 5.82E+04 | 1.28E−05 |
| | (SEQ ID NO: 90) | | | | | |
| P3A1-G3CP-BA11 | PGVQPAPGGGGS | 40146.51 | ND | 0.20 | 5.74E+04 | 1.14E−05 |
| | (SEQ ID NO: 90) | | | | | |
| BA11-G3CP-P3A1 | PGVQPAPGGGGS | 40146.51 | 40142.16 | 0.30 | 2.07E+05 | 6.15E−05 |
| | (SEQ ID NO: 90) | | | | | |
| BA11-P3A1-G3CP | PGVQPAPGGGGS | 40146.51 | ND | 0.14 | 2.94E+05 | 4.09E−05 |
| | (SEQ ID NO: 90) | | | | | |

Bi-paratopic binders show increased affinity for binding ROR1 as compared to the individual ROR1 binding monomers.

The constructs containing BA11 are examples of bi-paratopic bi-specific protein binders.

Furthermore, several bi-specific and bi-paratopic VNAR-based binders were developed by combining ROR1 loop-variant VNAR binders with the humanised VNAR BA11 or by combining different ROR1 loop-variant VNAR binders using a PGVQPCPGGGGGS (SEQ ID NO: 177) linker. This linker sequence also contains a single cysteine residue to facilitate site-selective bioconjugation of payloads to the proteins, in this linker, using thiol mediated chemical coupling strategies.

Proteins were expressed with a C-terminal tag QASGAHHHHHH (SEQ ID NO: 102) or QACKAHHHHHH (SEQ ID NO: 104) to aid purification and characterisation.

TABLE 12
Characterisation of bi-specific and bi-paratopic proteins containing
ROR1 VNAR loop library variants with cysteine containing linker sequences

| | | Expected | | | | |
|---|---|---|---|---|---|---|
| | | Mass | Observed | hROR1 binding (BLI) | | |
| Construct | Linker | (reduced); Da | Mass; Da | K_{D} (nM) | Kₐ (M⁻¹s⁻¹) | Kd (s⁻¹) |
| P3A1G1AE3- | PGVQPCPGGGGGS | 26232.9 | 26228.9 | 0.24 | 1.10E+06 | 2.60E−04 |
| G3CPG4 | (SEQ ID NO: 177) | | | | | |
| G3CPG4- | PGVQPCPGGGGGS | 26232.9 | 26228.8 | 0.28 | 6.41E+05 | 1.76E−04 |
| P3A1G1AE3 | (SEQ ID NO: 177) | | | | | |

Bi-specific VNAR binders were further modified through conjugation to the single cysteine residue in the linker sequence.

Prior to conjugation, 20 equivalents of TCEP were added to the bispecific proteins to remove cysteine/glutathione capping of the linker thiol. After incubation at room temperature for one hour the TCEP was removed by purification on a HiTrap SP cation exchange chromatography column (Cytiva). To load onto the column the protein was diluted three-fold in 50 mM Na Phosphate buffer pH 6.0. The protein was then eluted by an increasing gradient of elution buffer consisting of 50 nM Na phosphate pH 6.0, 1 M NaCl. To conjugate, 4 equivalents of a maleimide containing payload was added and left to incubate at room temperature for 1 hour. Free payload was then removed by cation exchange using the same protocol as above.

TABLE 13
Characterisation of bi-specific and bi-paratopic protein drug conjugates containing
ROR1 VNAR loop library variants with cysteine containing linker sequences

| | | Deglycosylated, | | DAR | Yield (overall) | hROR1 |
|---|---|---|---|---|---|---|
| | | reduced mass, Da | | by | Conjugate | binding (BLI) |
| Protein | Payload | Expected | Observed | MS | % | K_{D} (nM) |
| BA11- | MA-vc- | 26825.8 | 26823.3 | 1.0 | 35 | 0.54 |
| G3CP | PAB-MMAE | | | | | |
| G3CP G4- | MA-vc- | 27549.5 | 27544.9 | 1.0 | 28 | 0.76 |
| P3A1G1 | PAB-MMAE | | | | | |
| AE3 | | | | | | |

Without being bound by any particular theory, it is expected that conjugate yields can be improved by increasing scale of production and by employing optimised purification processes.

### Example 5—VNAR Reformatting as Fc Fusion Proteins

### VNAR Fc Fusion Proteins

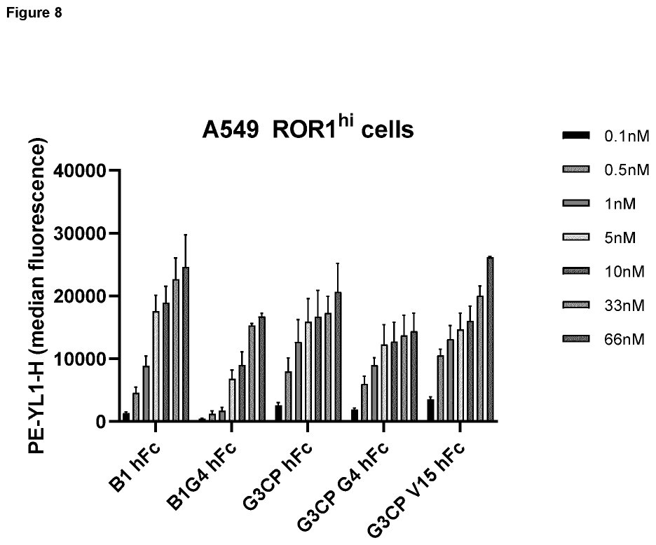

Fusion of proteins to an Fc domain can improve protein solubility and stability, markedly increase plasma half-life and improve overall therapeutic effectiveness. A human IgG1 Fc sequence is shown below and further examples are shown in FIG. 7.

Human IgG1 Fc (hFc)
(SEQ ID NO: 145)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

VNAR loop variants were genetically fused via standard [G4S]3 linkers to engineered hlgG1 Fc domains that contained a cysteine substitution in the hlgG1 Fc sequence, S239C (EU numbering). The VNAR Fc fusion proteins were transiently expressed as secreted protein in CHO K1 cells and purified from the media using MabSelect™ SuRe™ (Evitria, Switzerland). Purified proteins were exchanged into PBS pH 7.4 or PBS+100 mM Arg pH 7.4 and analysed by SEC (AdvanceBio, Agilent, running buffer DPBS pH 7.4), SDS PAGE and mass spectrometry to confirm sequence and protein integrity.

Binding kinetics were determined using a Pioneer Surface Plasmon Resonance (SPR) instrument (SensiQ/Pall ForteBio), or the Biolayer Interferometry (BLI) Octet K2 system (ForteBio). ROR1-hFc fusion proteins (extracelluar domains) were immobilised in sodium acetate pH5 buffer to COOH₂ chips or AR2G sensors using amine coupling. VNAR-Fc molecules were tested at various concentrations and the Ka (M⁻¹s⁻¹), Kd (s⁻¹) and K_{D}app (nM) values were determined using Octet Data Analysis High Throughput software (ForteBio) for Biolayer Interferometry. Alternatively, the kinetic parameters for binding were determined by immobilising the VNAR-hFc fusion onto AHC sensors. Human ROR1 (ECD) was tested at various concentrations and the Ka (M⁻¹s⁻¹), Kd (s⁻¹) and K_{D} (nM) values for 1:1 binding were determined using Octet Data Analysis High Throughput software (ForteBio) for Biolayer Interferometry. ROR1 2A2 mAb (Biolegend) was included as a control for positive/negative binding to ROR1. 2V is a control VNAR sequence, derived from a naïve VNAR library, so is representative of this protein class but has no known target. SEC analysis was performed as described previously. The data, summarised in Table 14, demonstrates advantageous properties of the loop library hFc variants versus the parental B1-hFc protein.

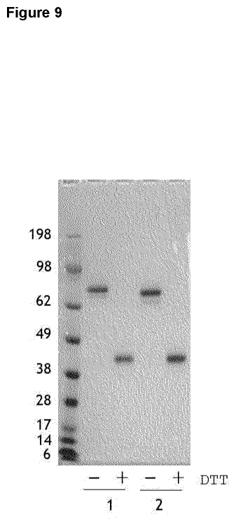

Binding of the VNAR-Fc fusions to ROR1 on the surface of cancer cell lines was measured by flow cytometry using the methods described previously, with binding of VNAR-hFc fusion molecules determined by adding 100 μL of PE-anti-human antibody (JIR) and incubating on ice for 30 mins. K_{D}app values were calculated from the increase in fluorescence intensity as a function of VNAR-hFc concentration. FIG. 8 shows the binding of different VNAR-Fc fusions to the ROR1^{hi} A549 lung adenocarcinoma cells.

Table 14—Summarises the expression yield (based on final purified, buffer exchanged protein), SEC analysis of the hFc fusions, the affinity of these molecules for ROR1 by BLI and the K_{Dapp} for binding ROR1 ^{hi} A549 cells.

TABLE 14
Characteristics of ROR1 loop variants VNAR-Fc fusions

| | B1- | G3CP- | G3CP G4- | 1H8- | 1H8 G4 - | 1H8 V15- |
|---|---|---|---|---|---|---|
| | hFc | hFc | hFc | hFc | hFc | hFc |
| Overall expression yield | 50 mg/L | >100 mg/L | >100 mg/L | 80 mg/L | >100 mg/L | >100 mg/L |
| (transient CHO expression) | | | | | | |
| % monomer by SEC | 81 | >98 | 95.2 | 90.5 | 96.1 | 92.5 |
| hROR1 affinity K_{D}app (nM) | <0.05 | <0.05 | <0.05 | <0.05 | 0.3 | 0.19 |
| hROR1 affinity K_{D} (1:1) (nM) | 3.78 | 5.74 | 17.9 | 26.9 | 2506 | 16.9 |
| ratROR1 affinity K_{D}app (nM) | 0.04 | 0.7 | 1.9 | 4.4 | NB | 2.5 |
| Cell surface hROR1 binding | 1.7 | 0.72 | 0.75 | ND | ND | ND |
| (flow cytometry titration | | | | | | |
| A549 cells) K_{D}app (nM) | | | | | | |

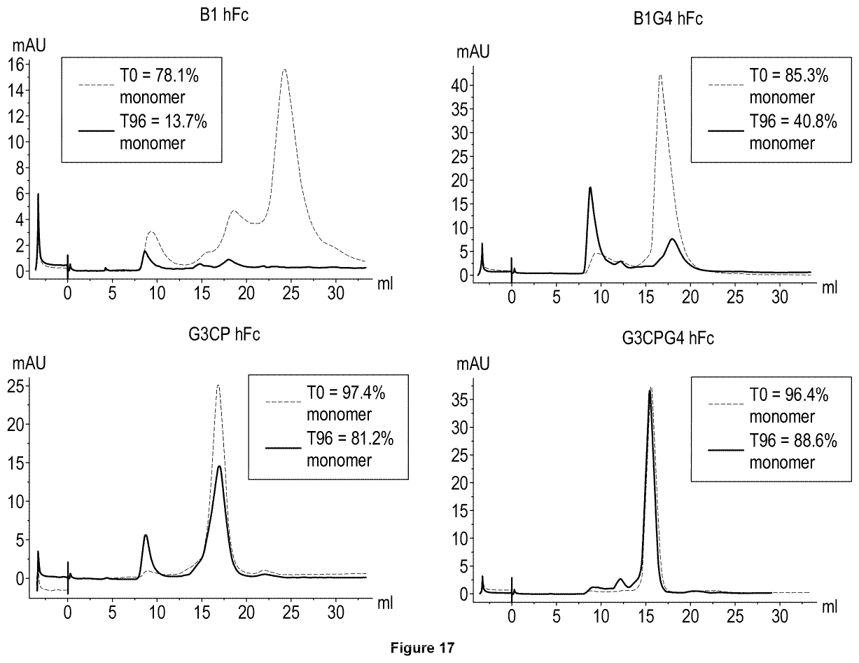

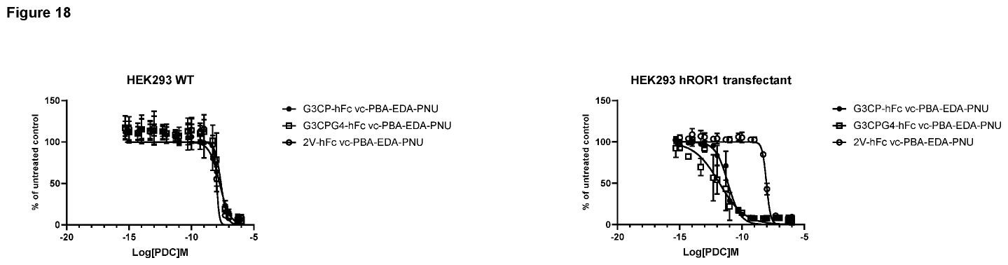

The relative stabilities of VNAR-hFc fusion proteins in PBS buffer were assessed. G3CP-hFc, G3CPG4-hFc and B1G4-hFc and parental B1-hFc were incubated at 2 mg/mL in sterile PBS buffer pH 7.4 containing 0.05% sodium azide at 37° C. for 96 h. The UV absorbance was measured at 280 nm and 320 nm at t=0 and t=96h. The monomericity of the proteins at t=0 and t=96 h was assessed by size-exclusion chromatography (S200 Increase 10/300GL with PBS pH 7.4 running buffer). As shown in FIG. 16 the UV absorbance at both 280 nm and 320 nm was increased after 96h incubation for B1-hFc but not for the loop library variants (i.e. G3CP-hFc and G3CPG4-hFc). The absorbance at 320 nm in particular is attributed to the scattering of light by aggregate particles. The protein concentrations for the loop library variants (i.e. G3CP-hFc and G3CPG4-hFc), calculated from the absorbance at 280 nm, remain constant throughout the experiment. The SEC analysis at t=0 versus t=96h (FIG. 17) shows that loop library variants (i.e. G3CP-hFc and G3CPG4-hFc) have good stability and are more stable than the parental protein B1-hFc.

### Bi-Paratopic VNAR Fc Fusion Proteins

VNAR loop variants were genetically fused via standard [G4S]3 linkers to hIgG1 Fcs engineered for heterodimerisation (Ridgway 1996 Protein Engineering 9(7):617-21). The Knob variant has a tryptophan substitution at position 336 (T366Y) and the Hole variant has a Threonine substitution at position 407 (Y407T) (EU numbering). This approach was used to generate bi-paratopic ROR1 binders where one arm comprises a VNAR loop variant and the other arm comprises a second ROR1 binding VNAR. In addition, a cysteine substitution was incorporated in the hIgG1 Fc sequence [S239C (EU numbering)] of both Knob and Hole variants to facilitate bioconjugation with different payloads.

The VNAR Fc fusion proteins were transiently co-expressed as secreted protein in CHO K1 cells and purified from the media using MabSelect™ SuRe™ (Evitria, Switzerland). Purified proteins were exchanged into PBS pH 7.4 and analysed by SEC (AdvanceBio, Agilent, running buffer DPBS), SDS PAGE and mass spectrometry to confirm sequence and protein integrity.

G3CP hFc(S239C + Y407T)
(SEQ ID NO: 146)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVNGGGGSGGGGGGGGSEPKSSDKTHTCPPCPAPELLGGPCVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLTSKLTVDKSRWQQGNVFSCSVMHEALHNHYTQKSLS
LSPGK
G3CPG4 hFc(S239C + Y407T)
(SEQ ID NO: 147)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIKGGGGSGGGGSGGGGSEPKSSDKTHTCPPCPAPELLGGPCVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLTSKLTVDKSRWQQGNVFSCSVMHEALHNHYTQKSL
SLSPGK
P3A1 hFc(S239C + T366Y)
(SEQ ID NO: 148)
TRVDQTPRTATKETGESLTINCVLTDTSYGLYSTSWFRKNPGTTDWERMS
IGGRYVESVNKGAKSFSLRIKDLTVADSATYYCKAREARHPWLRQWYDGA
GTVLTVNGGGGSGGGGSGGGGSEPKSSDKTHTCPPCPAPELLGGPCVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLYCLVKGFYPSDIAVEWESNGQPENNYKT
TPPVLDSDGSFFLYSKLTVDKSRWQQGNVFSCSVMHEALHNHYTQKSLSL
SPGK

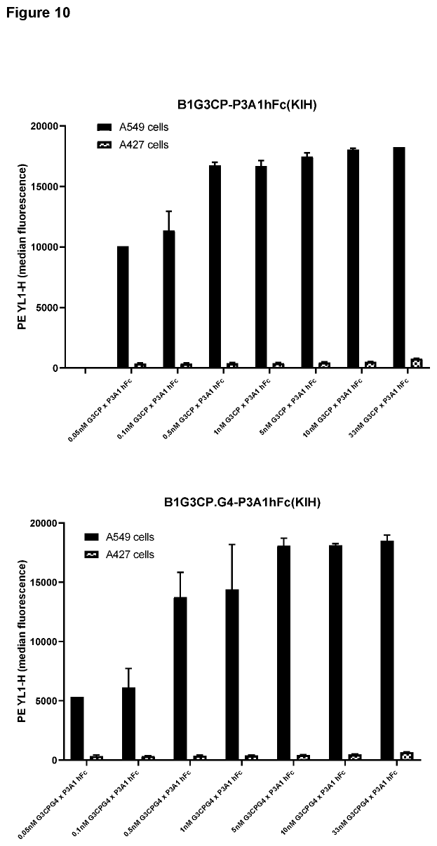

All proteins were characterised by reducing and non-reducing SDS PAGE analysis (FIG. 9) and mass spectrometry (Table 15).

Binding to ROR1 was determined using Biolayer interferometry (K2 Octet instrument/Pall ForteBio) as previously described. For BLI experiments ROR1-hFc, (extracelluar domain) was immobilised on the sensors. Data is shown in Table 15.

TABLE 15
MS characterisation of bi-paratopic VNAR-Fc fusions and binding to human ROR1 by BLI

| | | | | | | | ROR1 |
|---|---|---|---|---|---|---|---|
| | Expected MW ΔC | | | | | | binding |
| | term Lys, Da | | | Observed MW, Da | | | (BLI) |
| | monomer | monomer | | monomer | monomer | | K_{D} app |
| Protein | 1 | 2 | dimer | 1 | 2 | dimer | (nM) |
| G3CP-P3A1 hFc | 38,775.5 | 39,059.9 | 77,827.3 | 38,770.0 | 39,054.5 | 77,820.2 | <0.05 |
| (S239C + KIH) | | | | | | | |
| G3CPG4-P3A1 hFc | 38,766.4 | 39,059.9 | 77,818.2 | 38,760.9 | 39054.5 | 77,812.1 | <0.05 |
| (S239C + KIH) | | | | | | | |

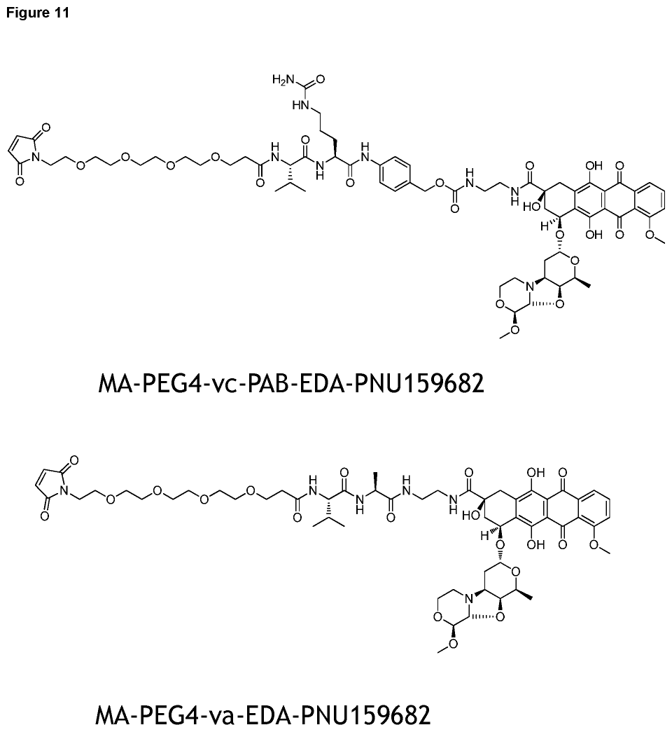

Binding of the bi-paratopic VNAR-Fc fusions to ROR1 on the surface of cancer cell lines was measured by flow cytometry using the methods described previously. Binding of VNAR-hFc fusion molecules determined by adding 100 μL of PE-anti-human antibody (JIR) and incubating on ice for 30 mins. K_{Dapp} values were calculated from the increase in fluorescence intensity as a function of VNAR-hFc concentration. FIG. 10 shows the binding of the bi-paratopic VNAR-Fc fusions to the ROR1^{hi} A549 lung adenocarcinoma cells and the ROR1^{low} A427 cells. G3CP-P3A1 hFc (S239C+KIH) and G3CPG4-P3A1 hFc (S239C+KIH) bind strongly to A549 cells with K_{D} app of 0.06 nM and 0.20 nM respectively but show little binding to A427 cells.

### Example 6—Loop Variant VNAR Drug Conjugates

### VNAR-hFc Drug Conjugates

Another approach for generating ADCs is to engineer cysteine substitutions or additions at positions on the light and heavy chains of antibodies and these cysteines provide reactive thiol groups for site specific labelling (Junutula 2008 Nature Biotechnology 26, 925-932, Jeffrey 2013, Sutherland 2016).

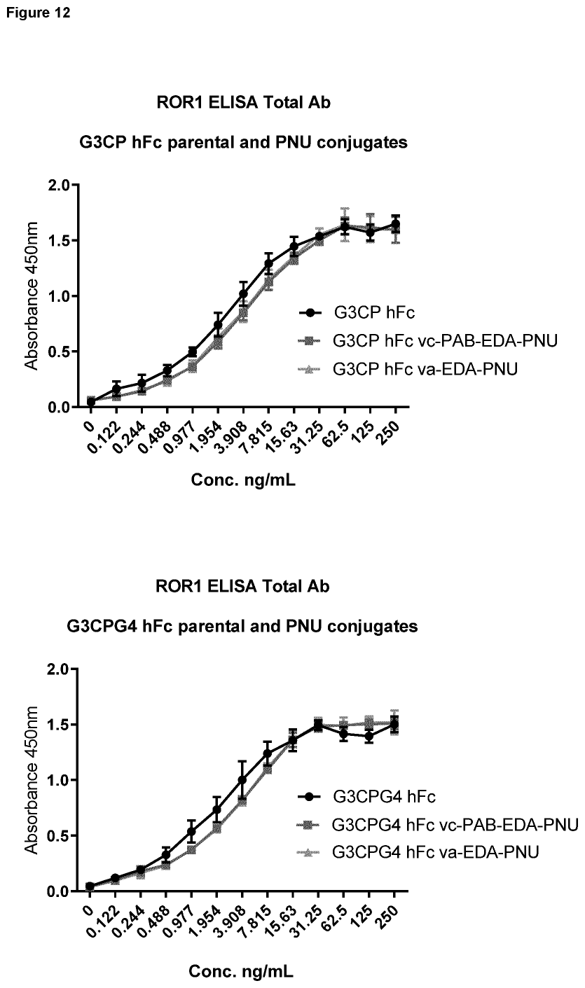

The anti ROR) loop library VNAR-hFc fusions were generated with an additional cysteine engineered into the Fc region as described previously, which enabled site specific labelling with maleimide derivatives of fluorescent labels (AF488) and cytotoxic drugs (MA PEG4 vc PAB EDA PNU 159682 and MA PEG4 va PAB EDA PNU 159682) (FIG. 11).

### Generation of VNAR-hFc—Drug Conjugates

Using a partial reduction, refolding and labelling method adapted from the literature [Junutula et al, 2008 Nat Biotech, Jeffrey et al, 2013 Bioconj Chem], these proteins were site specific labelled with the maleimide PNU derivatives. Briefly, 1 mg/mi VNAR hFc solutions were prepared in PBS+100 mM L-Arginine pH7.4 with 1 mM EDTA. 20 molar equivalents TCEP added and incubated at401 for a minimum of 48 hours. 30 molar equivalents DHAA added, pH adjusted to 6.5 and incubated at room temperature for 1 hour. Refolded VNAR Fc S239C was extensively dialysed or buffer exchanged into PBS+50 mM L-Arginine and quantified by UV before reacting with 4 or 5 molar equivalents maleimide PNU solution, room temperature overnight. Conjugates were purified by SEC and analysed by analytical HIC, analytical SEC, and LC-MS. Table 16 summaries the conjugates prepared.

TABLE 16
Summary of characteristics of VNAR-PNU conjugates

| | | Deglycosylated, | | DAR | Conjugate Yield | HMW |
|---|---|---|---|---|---|---|
| | | reduced mass, Da | | by | (overall) | aggregate |
| Protein | Payload | Expected | Observed | MS | % | % |
| G3CP | MA-PEG4-vc-PAB- | 40,239.6 | 40,239.8 | 2.1 | 52 | <3 |
| hFc | EDA-PNU159682 | | | | | |
| | MA-PEG4-va-EDA- | 40,003.6 | 40,005.3 | 2.0 | 61 | <3 |
| | PNU159682 | | | | | |
| G3CPG4 | MA-PEG4-vc-PAB- | 40,229.5 | 40,230.0 | 2.2 | 49 | <1.5 |
| hFc | EDA-PNU159682 | | | | | |
| | MA-PEG4-va-EDA- | 39,994.5 | 39,995.4 | 2.0 | 61 | <1 |
| | PNU159682 | | | | | |
| B1G4 | MA-PEG4-vc-PAB- | 40,253.0 | 40,251.8 | 2.0 | 37 | <1 |
| hFc | EDA-PNU159682 | | | | | |
| | MA-PEG4-va-EDA- | 40,017.0 | 40,016.2 | 2.0 | 43 | <1 |
| | PNU159682 | | | | | |
| B1 | MA-PEG4-vc-PAB- | 40,262.2 | 40,263.3 | 2.1 | 46 | <1 |
| hFc | EDA-PNU159682 | | | | | |
| | MA-PEG4-va-EDA- | 40,026.9 | 40,027.6 | 2.0 | 50 | <1 |
| | PNU159682 | | | | | |

SDS-PAGE and mass spectrometry analysis of the final conjugates determined that the labelling had proceeded in a quantitative fashion to give highly pure homogenous protein drug conjugates with drug to antibody ratio (DAR) of 2.

Binding of VNAR-hFc—Drug Conjugates to hROR1 by ELISA

The binding of G3CP hFc and G3CPG4 hFc and their respective drug conjugates to human ROR1 was assessed by ELISA. In brief, ELISA method as follows. Wells were coated with 100ng of ROR1-his antigen and incubated, covered, at room temperature for 2 hr. Plates washed 3× 400ul per well with PBST (PBS+0.05% Tween 20 (v/v)), then blocked with 4% skimmed milk powder (w/v) in PBST for 1 hour at 37° C. Plates washed as before plus additional wash in PBS alone. B1 loop variants (VNAR-hFc fusion) binding proteins were diluted in 4% milk PBST and incubated overnight at 4° C. Plates washed 3× with PBST, 3× PBS and binding detected using appropriate secondary detection antibody in 4% milk PBST, room temperature 1 hour. The secondary antibody used for detection was a Rabbit anti-human IgG H&L (HRP), Abcam Cat No. ab6759. Plates were washed 3× with PBST and then 100 μL TMB substrate (Thermo #34029) added and the reaction allowed to proceed at r.t. for 10 mins. 100 μL of 2M H₂SO₄ was then added to quench the reaction. The plate was centrifuged briefly before absorbance at 450 nm read on a CLARIOstar plate reader (BMG Labtech).

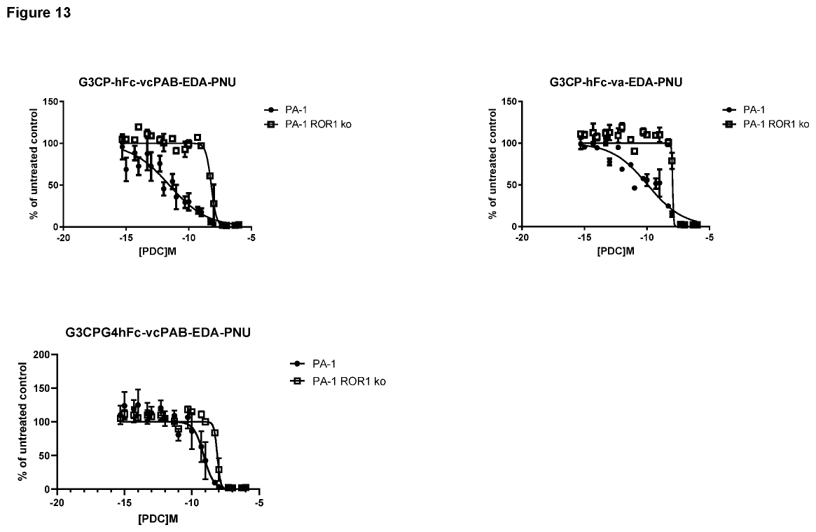

FIG. 12 shows that G3CP hFc and G3CPG4 hFc PNU conjugates bind strongly to human ROR1 and there is no loss in binding activity after conjugation of the different PNU linker payloads to the parental proteins.

In Vitro Cell Viability Assays for Cancer Cells Treated with Anti ROR1 VNAR Drug Conjugates

Cells were seeded into white, clear bottom 96 well plates (Costar) and incubated at 37° C., 5% CO₂ for 24 hours. On the following day, dilution series were set up for each test agent at ×10 working stocks. The dose response X10 stock was: 10000, 5000, 1000, 500, 100, 50, 10, 5, 1, 0.5 nM etc. 10 μL of the X10 stock solutions were added to the cell plates (90 μl per well) using a multichannel pipette. This resulted in a 1:10 dilution into the well and dose responses ranging from 1000 nM (column 1) to 0.05 nM (column 10) or continued to 0.5 fM, if required, for the most sensitive cells lines. 10 μl of vehicle control (PBS) was added to the control wells (columns 11 and 12). Plates were incubated at 37° C., 5% CO₂ for 72-96 hours. Promega Cell Titre Glo reagent was used as per the manufacturer's instructions to assess cell viability. Briefly, assay plates were removed from incubator and allowed to equilibrate to room temperature before adding 100 μl of room temperature Cell Titre Glo reagent to each 100 μl assay well. Plates were placed on a plate shaker for 2 minutes at 600 rpm. Plates were allowed to sit for a further 10 minutes at room temperature prior to measuring luminescence read-out using a Clariostar plate-reader (BMG). Data was analysed by calculating the average for untreated (vehicle only) control wells and determining the % of control for each treated well. % of control data was then plotted against Log [Treatment] concentration and the IC50 value derived using non-linear regression fitting in GraphPad Prism software.

The following cell lines were used
PA-1—human ovarian cancer cell line: EMEM, 10% hiFCS
PA-1 ROR1 ko—human ovarian cancer cell line with ROR1 knock-out: EMEM, 10% hiFCS
HEK293—human embryonic kidney cell line: EMEM, 10% FCS
HEK293 stably transfected with human ROR1 (HEK293.hROR1)—human embryonic kidney cell line stably expressing hROR1: EMEM, 10% FCS

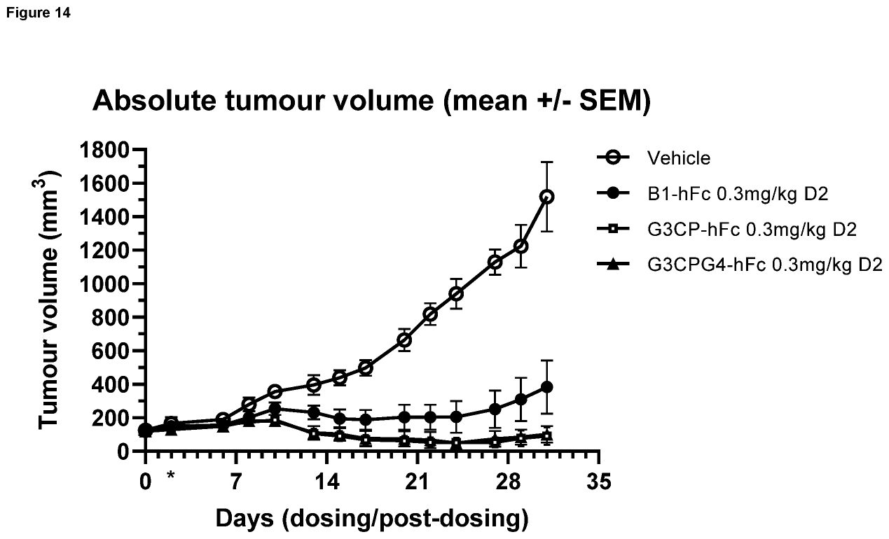

FIG. 13 shows dose response curves, with corresponding IC₅₀ values (Table 17), for cell-killing of the ROR1 positive PA-1 ovarian cancer cells and PA-1 ROR1 ko cells by G3CP-hFc-PNU conjugates (PEG4-vc PAB EDA PNU159682 and PEG4-va-EDA-PNU159682) and G3CPG4-hFc-PNU conjugate (PEG4-vc PAB EDA PNU159682). PA-1 ROR1 ko is PA-1 cancer cell-line where ROR1 expression has been knocked out.

TABLE 17
Calculated IC₅₀ values (nM) for the cell-killing of PA-1
and PA1 ROR1 ko cancer cells by G3CP-hFc conjugates.

| | IC₅₀ (nM) 96 hr | | |
|---|---|---|---|
| | | | PA-1/PA-1 |
| | PA-1 | PA-1 | ROR1 ko |
| | cells | ROR1 ko | window |
| G3CPhFc(S239C)- | 0.0028 | 6.3 | ×2250 |
| vcPAB-EDA-PNU | | | |
| G3CPhFc(S239C)- | 0.11 | 11.3 | ×103 |
| va-EDA-PNU | | | |
| G3CPG4hFc(S239C)- | 0.77 | 7.8 | ×10 |
| vcPAB-EDA-PNU | | | |

The ROR1 targeting VNAR-hFc conjugates show potent killing of PA-1 cell-lines, which is abrogated upon knockdown of the ROR1 receptor. There is >100 fold window in the IC₅₀ values for both of the G3CP-hFc PNU conjugates.

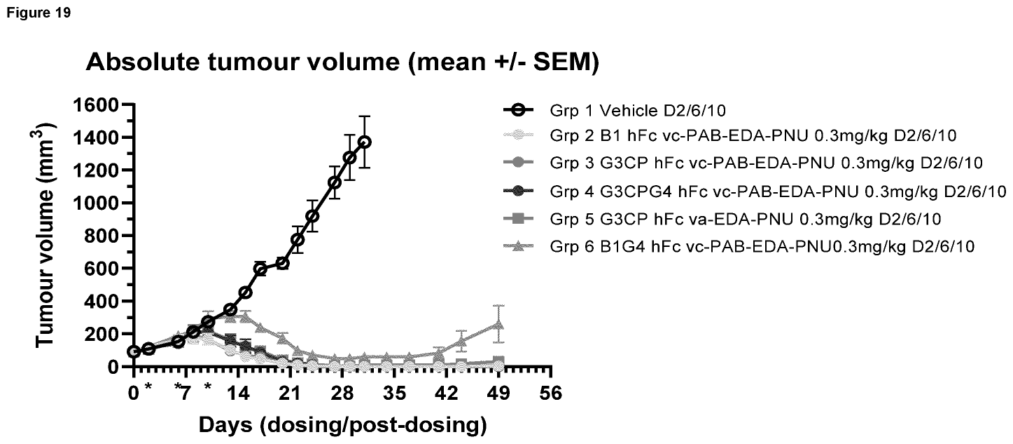

FIG. 18 shows dose response curves, with corresponding IC₅₀ values (Table 18), for cell-killing of the ROR1^{low} HEK293 cells and HEK293 cells stably transfected with human ROR1 (HEK293.hROR1) by G3CP-hFc-PNU, G3CPG4-hFc-PNU and 2V-hFc-PNU conjugates (PEG4-vc PAB EDA PNU159682). 2V is a control VNAR sequence, derived from a naïve VNAR library, so is representative of this protein class but has no known target.

TABLE 18
Calculated IC₅₀ values (nM) for the cell-killing of HEK293 WT and
HEK293.hROR1 cells by G3CP-hFc, G3CPG4-hFc and 2V-hFc conjugates.

| | IC₅₀ (nM) 96 hr | | |
|---|---|---|---|
| | HEK293 | | HEK293/HEK293.hROR1 |
| | cells | HEK293.hROR1 | window |
| G3CPhFc(S239C)- | 18.1 | 0.008 | ×2263 |
| vcPAB-EDA-PNU | | | |
| G3CPG4hFc(S239C)- | 23.5 | 0.002 | ×11750 |
| vcPAB-EDA-PNU | | | |
| 2VhFc(S239C)- | 10.6 | 9.2 | ×1.2 |
| vcPAB-EDA-PNU | | | |

The ROR1 targeting VNAR-hFc conjugates show potent killing of the HEK293.hROR1 cell-line, which is stably transfected with the ROR1 receptor, but not the ROR1^{low} wild-type HEK293 cells. There is >2000-fold window in the IC₅₀ values for both the G3CP-hFc PNU and G3CPG4-hFc-PNU conjugates for killing HEK293 vs HEK293.hROR1 cells but no window for the 2V-hFc-PNU non-binding control conjugate.

### Example 7—In Vivo Efficacy of Protein-Drug Conjugates in Patient-Derived Xenograft Model of Triple Negative Breast Cancer (TNBC)

An efficacy study in the ROR1+HBCx-28 patient-derived TNBC xenograft model was performed by XenTech (Paris).

Outbred athymic (nu/nu) female mice (HSD: Athymic Nude-Foxn1^{nu}) were implanted subcutaneously with tumours of the same in vivo passage. Mice were monitored until the tumour implants reached the study volume recruitment criteria of 60-200 mm³, preferably 75-196 mm³ in a sufficient number of animals. Mice were randomised to treatment groups such that there was no statistical difference between tumour volumes in each group. Randomisation was designated as Day 0 of the experiment. Mice were treated with vehicle or with the protein-drug conjugates B1-hFc-vc-PAB-EDA-PNU, B1G4-hFc-vc-PAB-EDA-PNU, G3CP-hFc-vc-PAB-EDA-PNU or G3CPG4-hFc-vc-PAB-EDA-PNU by single dose 0.3 mg/kg i.v. injection on day 2. All mice pre-primed with mouse IgG 20h before first PDC dose. Tumour volume was evaluated by measuring perpendicular tumour diameters, with a calliper, three times a week during the experimental period. Absolute tumour volume (ATV) was calculated using the formula TV (mm³)=[length (mm)×width (mm)²]×0.5, where the length and the width are the longest and the shortest perpendicular diameters of the tumour measured perpendicularly, respectively. All animals were weighed at the same time as tumour size measurement. Mice were observed and documented daily for changes in physical appearance, behaviour, adverse clinical signs and general welfare in line with local welfare and best veterinary practice guidelines.

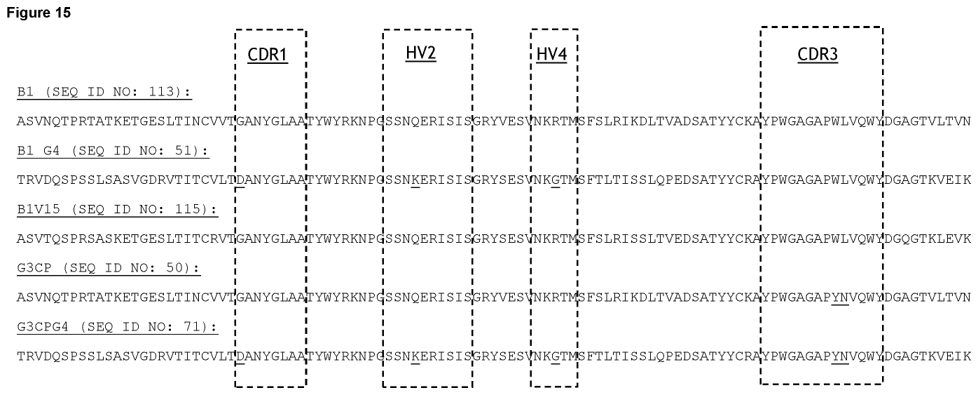

FIG. 14 shows the effect of the protein-drug conjugates on tumour growth versus vehicle control. All protein drug conjugates were well tolerated and show highly statistically significant in vivo efficacy in this ROR1+TNBC PDX model. B1G4-hFc-vc-PAB-EDA-PNU retains comparable levels of in vivo efficacy to B1-hFc-vc-PAB-EDA-PNU (data not shown). Loop library variants G3CP-hFc-vc-PAB-EDA-PNU and G3CPG4-hFc-vc-PAB-EDA-PNU show improved efficacy over the parental B1 fusion with complete and durable regressions observed for both loop library variants for the 0.3 mg/kg single dose regimen.

An efficacy study was also performed in the ROR1+HBCx-10 patient-derived TNBC xenograft model by XenTech (Paris).

Outbred athymic (nu/nu) female mice (HSD: Athymic Nude-Foxn1^{nu}) were implanted subcutaneously with tumours of the same in vivo passage. Mice were monitored until the tumour implants reached the study volume recruitment criteria of 75-196 mm³ in a sufficient number of animals. Mice were randomised to treatment groups such that there was no statistical difference between tumour volumes in each group. Randomisation was designated as Day 0 of the experiment. Mice were treated with vehicle or with the protein-drug conjugates B1-hFc-vc-PAB-EDA-PNU, B1G4-hFc-vc-PAB-EDA-PNU, G3CP-hFc-vc-PAB-EDA-PNU or G3CPG4-hFc-vc-PAB-EDA-PNU or G3CP-hFc-va-EDA-PNU at a dose of 0.3 mg/kg i.v. injection, three times, four days apart (3 ×Q4D on day 2, 6 and 10). All mice were pre-primed with mouse IgG 20h before first PDC dose. Tumour volume was evaluated by measuring perpendicular tumour diameters, with a calliper, three times a week during the experimental period. Absolute tumour volume (ATV) was calculated using the formula TV (mm³)=[length (mm)×width (mm)²]×0.5, where the length and the width are the longest and the shortest perpendicular diameters of the tumour measured perpendicularly, respectively. All animals were weighed at the same time as tumour size measurement. Mice were observed and documented daily for changes in physical appearance, behaviour, adverse clinical signs and general welfare in line with local welfare and best veterinary practice guidelines.

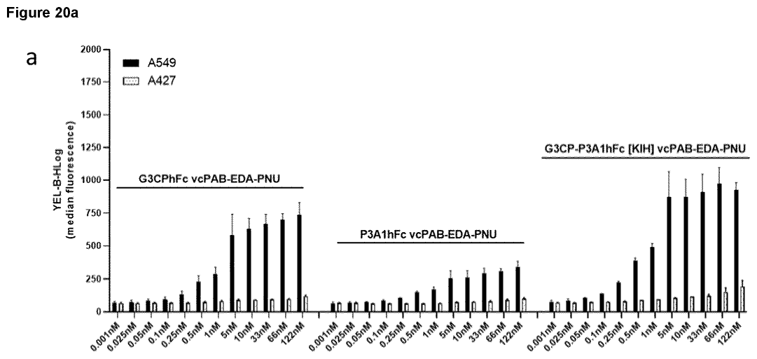

FIG. 19 shows the effect of the protein-drug conjugates on tumour growth versus vehicle control. All protein drug conjugates were well tolerated and show highly statistically significant in vivo efficacy in this ROR1+TNBC PDX model with complete and durable regressions observed for this dosing regimen.

### Example 8—Bi-Paratopic Loop Variant VNAR Drug Conjugates

### Bi-Paratopic VNAR-hFc Drug Conjugates

Bi-paratopic anti-ROR loop library VNAR-hFc fusions, as described in Example 5, were generated with an additional cysteine engineered into the Fc region as described previously, which enabled site specific labelling with maleimide derivatives of labels and cytotoxic drugs.

### Generation of Bi-Paratopic VNAR-hFc—Drug Conjugates

Bi-paratopic ROR binding proteins G3CP-P3A1 hFc (S239C+KIH) and G3CPG4-P3A1 hFc (S239C+KIH) were conjugated with MC-vc-PAB-MMAE or MA-PEG4-vc-PAB-EDA-PNU159682 using a partial reduction, refolding and labelling method as described in Example 6. Conjugates were purified by SEC and analysed by analytical HIC, analytical SEC, and LC-MS. Table 19 summaries the conjugates prepared.

TABLE 19
Summary of characteristics of Bi-paratopic VNAR-PNU conjugates

| | | Deglycosylated, | | DAR | Conjugate Yield | HMW |
|---|---|---|---|---|---|---|
| | | reduced mass, Da | | by | (overall) | aggregate |
| Protein | Payload | Expected | Observed | MS | % | % |
| G3CP-P3A1 hFc | MA-PEG4-vc-PAB- | 40,178.0 & | 40,180.7 & | 2.0 | 50 | <1 |
| (S239C + KIH) | EDA-PNU159682 | 40,462.4 | 40,465.1 | | | |
| G3CP-P3A1 hFc | MC-vc-PAB-MMAE | 40,091.3 & | 40,087.3 & | 2.0 | 63 | <1 |
| (S239C + KIH) | | 39,059.9 | 40,371.7 | | | |
| G3CPG4-P3A1 hFc | MA-PEG4-vc-PAB- | 40,168.9 & | 40,170.9 & | 2.15 | 44 | <1 |
| (S239C + KIH) | EDA-PNU159682 | 40,462.4 | 40,464.8 | | | |
| G3CPG4-P3A1 hFc | MC-vc-PAB-MMAE | 40,082.2 & | 40,077.7 & | 2.0 | 65 | <1 |
| (S239C + KIH) | | 40,375.7 | 40,371.3 | | | |
| P3A1 hFc | MA-PEG4-vc-PAB- | 40,398.8 | 40,402.6 | 2.16 | 60 | <1 |
| (S239C) | EDA-PNU159682 | | | | | |

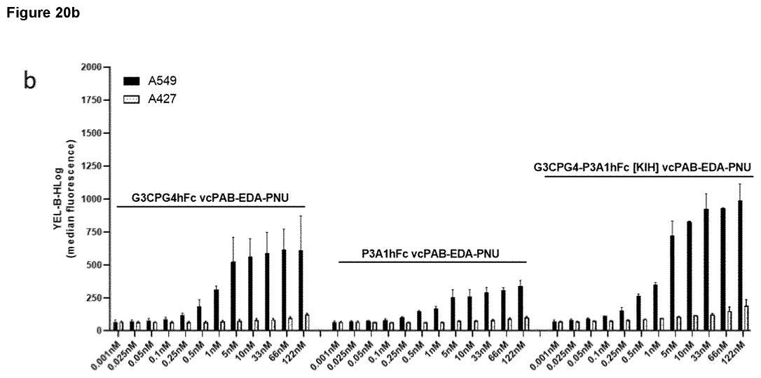

Binding of the bi-paratopic VNAR-Fc-PNU conjugates to ROR1 on the surface of cancer cell lines was measured by flow cytometry using the methods described previously. Binding of VNAR-hFc-PNU molecules was determined by adding 100 μL of PE-anti-human antibody (JIR) and incubating on ice for 30 mins. K_{Dapp} values were calculated from the increase in fluorescence intensity as a function of VNAR-hFc concentration. FIG. 20 a and b shows the binding of the bi-paratopic VNAR-Fc-PNU conjugates (PEG4-vc PAB EDA PNU159682) to the ROR1^{hi} A549 lung adenocarcinoma cells and the ROR1^{low} A427 cells along with the corresponding mono-paratopic PNU conjugates. G3CP-P3A1 hFc (S239C+KIH)—PNU and G3CPG4-P3A1 hFc (S239C+KIH)—PNU bind strongly to A549 cells with K_{D} app of 0.92 nM and 1.83 nM respectively but show little binding to A427 cells. G3CP-P3A1 hFc (S239C+KIH)—PNU demonstrates a greater level of saturation binding to A549 cells as compared to the corresponding G3CP-hFc-PNU and P3A1-hFc-PNU conjugates (FIG. 20a). Similarly, G3CPG4-P3A1 hFc (S239C+KIH)—PNU demonstrates a greater level of saturation binding to A549 cells as compared to the corresponding G3CPG4-hFc-PNU and P3A1-hFc-PNU conjugates (FIG. 20b)

In Vitro Cell Viability Assays for Cancer Cells Treated with Anti ROR1 Bi-Paratopic VNAR Drug Conjugates

Cell Titre Glo assays were performed as describe din Example 6. Cells were incubated with VNAR-hFc conjugates at 37° C., 5% CO₂ for 96 hours and the % of cell viability determined as a function of dose response. The % of control data was plotted against Log [Treatment] concentration and the IC₅₀ value derived using non-linear regression fitting in GraphPad Prism software.

The following cell lines were used
PA-1—human ovarian cancer cell line: EMEM, 10% hiFCS
PA-1 ROR1 ko—human ovarian cancer cell line with ROR1 knock-out: EMEM, 10% hiFCS
Kasumi-2—human B cell precursor leukaemia cell line: RPMI 1640, 10% hiFCS
MHH-ES1—human Ewings sarcoma cell line: RPMI 1640, 10% hiFCS

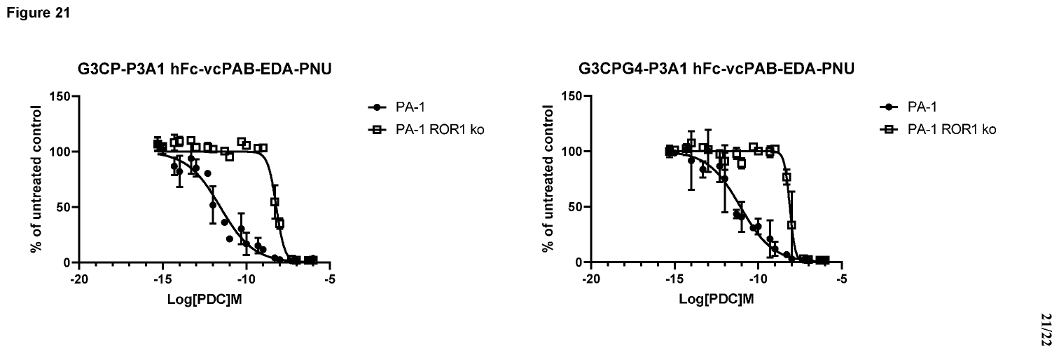

FIG. 21 shows dose response curves for cell-killing of the ROR1 positive PA-1 ovarian cancer cells and PA-1 ROR1 ko cells by G3CP-P3A1 hFc (S239C+KIH)—PNU and G3CPG4-P3A1 hFc (S239C+KIH)—PNU conjugates (PEG4-vc PAB EDA PNU159682). PA-1 ROR1 ko is PA-1 cancer cell-line where ROR1 expression has been knocked out.

Table 20 shows IC₅₀ values, for cell-killing of Kasumi-2, MHH-ES1, PA-1 and PA-1 ROR1 ko cells by G3CP-P3A1 hFc (S239C+KIH)—PNU and G3CPG4-P3A1 hFc (S239C+KIH)—PNU conjugates (PEG4-vc PAB EDA PNU159682). The cell-surface ROR1 receptor number was determined for each cell-line by flow cytometry using BD Biosciences Quantibrite beads.

TABLE 20
Calculated IC₅₀ values (nM) for the cell-killing of PA-1
and PA1 ROR1 ko cancer cells by G3CP-P3A1 hFc (S239C +
KIH)-PNU and G3CPG4-P3A1 hFc (S239C + KIH)-PNU conjugates.

| | IC50 (nM) | | | |
|---|---|---|---|---|
| | Kasumi-2 | MHH-ES1 | PA1 | PA1.ROR1ko |
| ROR1 receptor/cell | 5,086 | 2,342 | 12,249 | 0 |
| G3CP-P3A1 hFc | 0.016 | 0.3 | 0.003 | 6.3 |
| (S239C + KIH) | | | | |
| vcPAB-EDA-PNU | | | | |
| G3CPG4-P3A1 hFc | 0.141 | 1 | 0.009 | 7.8 |
| (S239C + KIH) | | | | |
| vcPAB-EDA-PNU | | | | |

The ROR1 targeting bi-paratopic VNAR-hFc conjugates show potent killing of the ROR1+ cancer cell-lines, but not the ROR1 negative PA1.ROR1 ko cell-line.

### Example 9—In Vivo Efficacy of Bi-Paratopic Protein-Drug Conjugates in Patient-Derived Xenograft Model of Triple Negative Breast Cancer (TNBC)

An efficacy study in the ROR1+HBCx-28 patient-derived TNBC xenograft model was performed by XenTech (Paris).

Outbred athymic (nu/nu) female mice (HSD: Athymic Nude-Foxn1^{nu}) were implanted subcutaneously with tumours of the same in vivo passage. Mice were monitored until the tumour implants reached the study volume recruitment criteria of 100-200 mm³ in a sufficient number of animals. Mice were randomised to treatment groups such that there was no statistical difference between tumour volumes in each group. Randomisation was designated as Day 0 of the experiment. Mice were treated with vehicle or with the Bi-paratopic protein-drug conjugates G3CP-P3A1 hFc (S239C+KIH)-vc-PAB-EDA-PNU and G3CPG4-P3A1 hFc (S239C+KIH) vc-PAB-EDA-PNU either by single dose 0.3 mg/kg i.v. injection on day 2 or by 3×0.1 mg/kg i.v. injections four days apart (3×Q4D on day 2, 6 and 10). All mice were pre-primed with mouse IgG 20h before first PDC dose. Tumour volume was evaluated by measuring perpendicular tumour diameters, with a caliper, three times a week during the experimental period. Absolute tumour volume (ATV) was calculated using the formula TV (mm³)=[length (mm)×width (mm)²]×0.5, where the length and the width are the longest and the shortest perpendicular diameters of the tumour measured perpendicularly, respectively. All animals were weighed at the same time as tumour size measurement. Mice were observed and documented daily for changes in physical appearance, behaviour, adverse clinical signs and general welfare in line with local welfare and best veterinary practice guidelines.

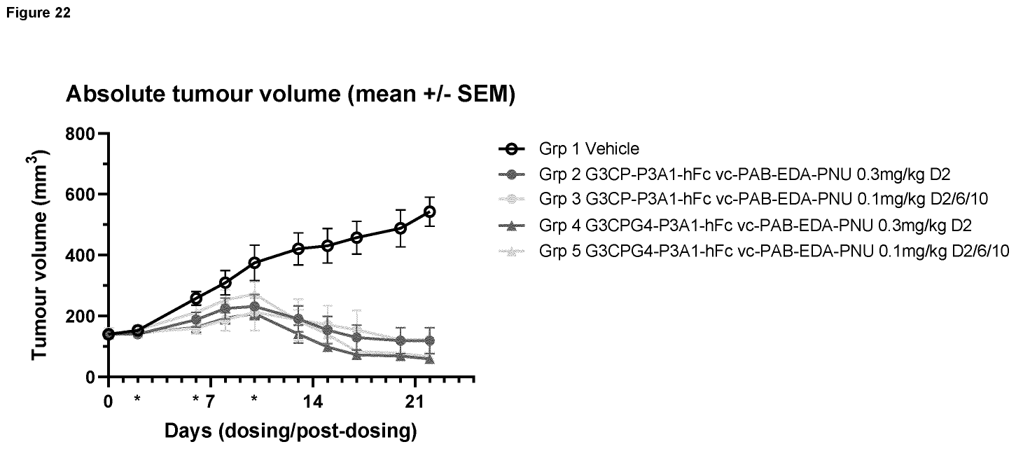

FIG. 22 shows the effect of the protein-drug conjugates on tumour growth versus vehicle control. All protein drug conjugates were well tolerated and both bi-paratopic loop library variants G3CP-P3A1-hFc-vc-PAB-EDA-PNU and G3CPG4-P3A1-hFc-vc-PAB-EDA-PNU show excellent in vivo efficacy in this ROR1+TNBC PDX model, with tumour regressions observed for both agents.

### Example 10—ROR1 VNAR Bi-Specifics

Bispecific target combinations for ROR1 binding VNARs include, for example, HSA for half-life extension; bispecific engagement of ROR1 and serum albumin RTKs e.g. EGFR, Her3; bispecific targeting both EGFR and ROR1 or HER3 and ROR1 on the surface of cells.

The VNAR BA11, already discussed and exemplified herein, is an example of a HSA-binding VNAR. Bi-specific molecules comprising a HSA-binding VNAR (such as BA11) and another specific binding molecule are discussed.

ROR1×CD3 bispecific sequences combining N-terminal ROR1 VNARs with a C-terminal anti-CD3 scFv (clone OKT3) via 2 different length G4S linkers were expressed in CHO cells (Evitria) and purified by IMAC (HisTrap Excel, GE Healthcare) followed by SEC (Superdex 200 26/60, GE Healthcare). Similarly, biparatopic ROR1×CD3 bispecific sequences combining N-terminal biparatopic ROR1 VNARs with the C-terminal anti-CD3 scFv were also expressed in CHO (Evitria).

CD3 BiTE-like approach; examples of CD3 binding sequences for use as an ROR1 VNAR bispecific Anti CD3 scFv clone OKT3 (WO 2014028776 Zyngenia) and orientation and humanised derivatives thereof

VH-[G₄S]₃-VL
(SEQ ID NO: 149)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSDIQLTQSPAIMSA
SPGEKVTMTCRASSSVSYMNWYQQKSGTSPKRWIYDTSKVASGVPYRFS
GSGSGTSYSLTISSMEAEDAATYYCQQWSSNPLTFGAGTKLELKS

Humanised anti CD3 scFv UCHT1 (Arnett et al PNAS 2004 101(46) 16268-16273) and derivatives thereof

VL-[G₄S]₃-VH
(SEQ ID NO: 150)
MDIQMTQTTSSLSASLGDRVTISCRASQDIRNYLNWYQQKPDGTVKLLIY
YTSRLHSGVPSKFSGSGSGTDYSLTISNLEQEDIATYFCQQGNTLPWTFA
GGTKLEIKGGGGSGGGGSGGGGSEVQLQQSGPELVKPGASMKISCKASGY
SFTGYTMNWVKQSHGKNLEWMGLINPYKGVSTYNQKFKDKATLTVDKSSS
TAYMELLSLTSEDSAVYYCARSGYYGDSDWYFDVWGQGTTLTVFS

### Example 11—ROR1 CAR-T approaches

Chimeric antigen receptors (CARs) based on the ROR1-specific antigen binding molecules described in the present application may be generated. Furthermore, engineered T cells expressing such a CAR may also be generated, which may then be used in, for example, adoptive cell therapy.

In brief, a nucleic acid construct encoding a ROR1-specific CAR may be produced. The ROR1-specific CAR may include an intracellular activation domain, a transmembrane domain, and an extracellular domain comprising the ROR1-specific antigen binding molecule described herein. The nucleic acid construct may then be incorporated into a viral vector, such as a retroviral vector (e.g., a lentiviral vector).

T cells may be isolated from a patient in need of treatment, which may then be modified to express the nucleic acid construct encoding the CAR, for example by retroviral transfection or gene-editing using approaches such as CRISPR-CAS-9.

The engineered T cells may then be re-infused into the patient in order to treat the condition, such as treatment of cancer.

1-114. (canceled)

115. A receptor tyrosine kinase-like orphan receptor 1 (ROR1) specific antigen binding molecule comprising an amino acid sequence represented by the formula (I):
FW1-CDR1-FW2-HV2-FW3a-HV4-FW3b-CDR3-FW4 (I)
wherein
CDR3 is a CDR sequence having an amino acid sequence selected from the group consisting of
(SEQ ID NO: 23)
YPWGAGAPYNVQWY,
(SEQ ID NO: 20)
YPWGAGAPYLVQWY,
(SEQ ID NO: 24)
YPWGAGAPWNVQWY,
(SEQ ID NO: 11)
YPSGAGAPRPVQWY,
(SEQ ID NO: 12)
YPWGAGAPCLVQWY,
(SEQ ID NO: 13)
YPWGAGAPRLVQWY,
(SEQ ID NO: 14)
YPWGAGAPRQVQWY,
(SEQ ID NO: 15)
YPWGAGAPRSVQWY,
(SEQ ID NO: 16)
YPWGAGAPSLVQWY,
(SEQ ID NO: 17)
YPWGAGAPSNVQWY,
(SEQ ID NO: 18)
YPWGAGAPSQVQWY,
(SEQ ID NO: 19)
YPWGAGAPSSVQWY,
(SEQ ID NO: 21)
YPWGAGAPWQVQWY,
(SEQ ID NO: 22)
YPWGAGAPWSVQWY,
and
(SEQ ID NO: 10)
YPWGAGAPWLVQWY;
CDR1 is a CDR sequence having an amino acid sequence selected from the group consisting of
(SEQ ID NO: 1)
GANYGLAA,
(SEQ ID NO: 5)
DANYGLAA,
(SEQ ID NO: 2)
GANYDLSA,
(SEQ ID NO: 3)
GANYGLSA,
and
(SEQ ID NO: 4)
GANYDLAA
FW1 is a framework region;
FW2 is a framework region;
HV2 is a hypervariable sequence having an amino acid sequence selected from the group consisting of SSNQERISIS (SEQ ID NO: 6), and SSNKERISIS (SEQ ID NO: 7);
FW3a is a framework region;
HV4 is a hypervariable sequence having an amino acid sequence selected from the group consisting of NKRTM (SEQ ID NO: 8) and NKGTM (SEQ ID NO: 9);
FW3b is a framework region;
FW4 is a framework region;
wherein if CDR3 is YPWGAGAPWLVQWY (SEQ ID NO: 10) then CDR1 is selected from the group consisting of DANYGLAA (SEQ ID NO: 5), GANYGLSA (SEQ ID NO: 3) and GANYDLAA (SEQ ID NO: 4).

116. The ROR1-specific antigen binding molecule of claim 115, wherein
FW1 is a framework region of from 20 to 28 amino acids;
FW2 is a framework region of from 6 to 14 amino acids;
FW3a is a framework region of from 6 to 10 amino acids;
FW3b is a framework region of from 17 to 24 amino acids; and/or
FW4 is a framework region of from 7 to 14 amino acids,
optionally wherein
FW1 has an amino acid sequence selected from the group consisting of:
ASVNQTPRTATKETGESLTINCVVT (SEQ ID NO: 40), TRVDQSPSSLSASVGDRVTITCVLT
(SEQ ID NO: 41) and ASVTQSPRSASKETGESLTITCRVT (SEQ ID NO: 42), or a functional variant thereof with a sequence identity of at least 45%;
FW2 has an amino acid sequence according to TYWYRKNPG (SEQ ID NO: 43), or a functional variant thereof with a sequence identity of at least 45%;
FW3a has an amino acid sequence selected from the group consisting of: GRYVESV (SEQ ID NO: 44) and GRYSESV (SEQ ID NO: 45), or a functional variant thereof with a sequence identity of at least 45%;
FW3b has an amino acid sequence selected from the group consisting of:
SFSLRIKDLTVADSATYYCKA (SEQ ID NO: 84), SFTLTISSLQPEDSATYYCRA (SEQ ID NO: 46) and SFSLRISSLTVEDSATYYCKA (SEQ ID NO: 47), or a functional variant thereof with a sequence identity of at least 45%;
and/or
FW4 has an amino acid sequence selected from the group consisting of: DGAGTVLTVN (SEQ ID NO: 48), DGAGTKVEIK (SEQ ID NO: 49) or DGQGTKLEVK (SEQ ID NO: 85) or a functional variant thereof with a sequence identity of at least 45%.

117. The ROR1-specific antigen binding molecule of claim 115, wherein the ROR1-specific antigen binding molecule comprises an amino acid sequence selected from the group consisting of:
a)
(SEQ ID NO: 50)
ASVNQTPRTATKETGESLTINCVVTGANYGLAATYWYRKNPGSSNQERIS
ISGRYVESVNKRTMSFSLRIKDLTVADSATYYCKAYPWGAGAPYNVQWYD
GAGTVLTVN;
b)
(SEQ ID NO: 51)
TRVDQSPSSLSASVGDRVTITCVLTDANYGLAATYWYRKNPGSSNKERIS
ISGRYSESVNKGTMSFTLTISSLQPEDSATYYCRAYPWGAGAPWLVQWYD
GAGTKVEIK;
c)
(SEQ ID NO: 52)
ASVNQTPRTATKETGESLTINCVVTGANYDLSATYWYRKNPGSSNQERIS
ISGRYVESVNKRTMSFSLRIKDLTVADSATYYCKAYPSGAGAPRPVQWYD
GAGTVLTVN;
d)
(SEQ ID NO: 53)
ASVNQTPRTATKETGESLTINCVVTGANYGLAATYWYRKNPGSSNQERIS
ISGRYVESVNKRTMSFSLRIKDLTVADSATYYCKAYPWGAGAPCLVQWYD
GAGTVLTVN;
e)
(SEQ ID NO: 54)
ASVNQTPRTATKETGESLTINCVVTGANYGLAATYWYRKNPGSSNQERIS
ISGRYVESVNKRTMSFSLRIKDLTVADSATYYCKAYPWGAGAPRLVQWYD
GAGTVLTVN;
f)
(SEQ ID NO: 55)
ASVNQTPRTATKETGESLTINCVVTGANYGLAATYWYRKNPGSSNQERIS
ISGRYVESVNKRTMSFSLRIKDLTVADSATYYCKAYPWGAGAPRQVQWYD
GAGTVLTVN;,
g)
(SEQ ID NO: 56)
ASVNQTPRTATKETGESLTINCVVTGANYGLSATYWYRKNPGSSNQERIS
ISGRYVESVNKRTMSFSLRIKDLTVADSATYYCKAYPWGAGAPRSVQWYD
GAGTVLTVN;
h)
(SEQ ID NO: 57)
ASVNQTPRTATKETGESLTINCVVTGANYGLAATYWYRKNPGSSNQERIS
ISGRYVESVNKRTMSFSLRIKDLTVADSATYYCKAYPWGAGAPRSVQWYD
GAGTVLTVN;
i)
(SEQ ID NO: 58)
ASVNQTPRTATKETGESLTINCVVTGANYGLAATYWYRKNPGSSNQERIS
ISGRYVESVNKRTMSFSLRIKDLTVADSATYYCKAYPWGAGAPSLVQWYD
GAGTVLTVN;
j)
(SEQ ID NO: 59)
ASVNQTPRTATKETGESLTINCVVTGANYGLSATYWYRKNPGSSNQERIS
ISGRYVESVNKRTMSFSLRIKDLTVADSATYYCKAYPWGAGAPSNVQWYD
GAGTVLTVN;
k)
(SEQ ID NO: 60)
ASVNQTPRTATKETGESLTINCVVTGANYGLAATYWYRKNPGSSNQERIS
ISGRYVESVNKRTMSFSLRIKDLTVADSATYYCKAYPWGAGAPSQVQWYD
GAGTVLTVN;
l)
(SEQ ID NO: 61)
ASVNQTPRTATKETGESLTINCVVTGANYGLAATYWYRKNPGSSNQERIS
ISGRYVESVNKRTMSFSLRIKDLTVADSATYYCKAYPWGAGAPSSVQWYD
GAGTVLTVN;
m)
(SEQ ID NO: 62)
ASVNQTPRTATKETGESLTINCVVTGANYGLSATYWYRKNPGSSNQERIS
ISGRYVESVNKRTMSFSLRIKDLTVADSATYYCKAYPWGAGAPWLVQWYD
GAGTVLTVN;
n)
(SEQ ID NO: 63)
ASVNQTPRTATKETGESLTINCVVTGANYDLAATYWYRKNPGSSNQERIS
ISGRYVESVNKRTMSFSLRIKDLTVADSATYYCKAYPWGAGAPWLVQWYD
GAGTVLTVN;
o)
(SEQ ID NO: 64)
ASVNQTPRTATKETGESLTINCVVTGANYGLAATYWYRKNPGSSNQERIS
ISGRYVESVNKRTMSFSLRIKDLTVADSATYYCKAYPWGAGAPWNVQWYD
GAGTVLTVN;
p)
(SEQ ID NO: 65)
ASVNQTPRTATKETGESLTINCVVTGANYGLAATYWYRKNPGSSNQERIS
ISGRYVESVNKRTMSFSLRIKDLTVADSATYYCKAYPWGAGAPWQVQWYD
GAGTVLTVN;
q)
(SEQ ID NO: 66)
ASVNQTPRTATKETGESLTINCVVTGANYGLSATYWYRKNPGSSNQERIS
ISGRYVESVNKRTMSFSLRIKDLTVADSATYYCKAYPWGAGAPWQVQWYD
GAGTVLTVN;
r)
(SEQ ID NO: 67)
ASVNQTPRTATKETGESLTINCVVTGANYDLAATYWYRKNPGSSNQERIS
ISGRYVESVNKRTMSFSLRIKDLTVADSATYYCKAYPWGAGAPWQVQWYD
GAGTVLTVN;
s)
(SEQ ID NO: 68)
ASVNQTPRTATKETGESLTINCVVTGANYDLAATYWYRKNPGSSNQERIS
ISGRYVESVNKRTMSFSLRIKDLTVADSATYYCKAYPWGAGAPWSVQWYD
GAGTVLTVN;
t)
(SEQ ID NO: 69)
ASVNQTPRTATKETGESLTINCVVTGANYGLAATYWYRKNPGSSNQERIS
ISGRYVESVNKRTMSFSLRIKDLTVADSATYYCKAYPWGAGAPWSVQWYD
GAGTVLTVN;
u)
(SEQ ID NO: 70)
ASVNQTPRTATKETGESLTINCVVTGANYGLSATYWYRKNPGSSNQERIS
ISGRYVESVNKRTMSFSLRIKDLTVADSATYYCKAYPWGAGAPWSVQWYD
GAGTVLTVN;
v)
(SEQ ID NO: 71)
TRVDQSPSSLSASVGDRVTITCVLTDANYGLAATYWYRKNPGSSNKERIS
ISGRYSESVNKGTMSFTLTISSLQPEDSATYYCRAYPWGAGAPYNVQWYD
GAGTKVEIK;
w)
(SEQ ID NO: 72)
ASVTQSPRSASKETGESLTITCRVTGANYGLAATYWYRKNPGSSNQERIS
ISGRYSESVNKRTMSFSLRISSLTVEDSATYYCKAYPWGAGAPYNVQWYD
GQGTKLEVK;
x)
(SEQ ID NO: 73)
TRVDQSPSSLSASVGDRVTITCVLTDANYGLAATYWYRKNPGSSNKERIS
ISGRYSESVNKGTMSFTLTISSLQPEDSATYYCRAYPWGAGAPSSVQWYD
GAGTKVEIK;
y)
(SEQ ID NO: 74)
ASVTQSPRSASKETGESLTITCRVTGANYGLAATYWYRKNPGSSNQERIS
ISGRYSESVNKRTMSFSLRISSLTVEDSATYYCKAYPWGAGAPSSVQWYD
GQGTKLEVK;
z)
(SEQ ID NO: 75)
TRVDQSPSSLSASVGDRVTITCVLTDANYGLAATYWYRKNPGSSNKERIS
ISGRYSESVNKGTMSFTLTISSLQPEDSATYYCRAYPWGAGAPRQVQWYD
GAGTKVEIK;
and
aa)
(SEQ ID NO: 76)
ASVTQSPRSASKETGESLTITCRVTGANYGLAATYWYRKNPGSSNQERIS
ISGRYSESVNKRTMSFSLRISSLTVEDSATYYCKAYPWGAGAPRQVQWYD
GQGTKLEVK;
or a functional variant having CDR1, HV2, HV4 and CDR2 sequences according to any thereof and having FW1, FW2, FW3a, FW3b and FW4 sequences having a combined sequence identity of at least 45% to the combined FW1, FW2, FW3a, FW3b and FW4 sequences of any thereof.

118. The ROR1-specific antigen binding molecule of claim 115, wherein the ROR1-specific antigen binding molecule:
(a) does not bind to receptor tyrosine kinase-like orphan receptor 2 (ROR2),
(b) binds to both human ROR1 and murine ROR1 (mROR1),
(c) binds to deglycosylated ROR1;
(d) is humanized;
(e) is de-immunized
(f) is conjugated to a detectable label, dye, toxin, drug, pro-drug, radionuclide or biologically active molecule.
(g) selectively interacts with ROR1 protein with an affinity constant of approximately 0.01 to 50 nM, preferably 0.1 to 30 nM, even more preferably 0.1 to 10 nM, and/or
(h) is capable of mediating killing of ROR1-expressing tumour cells, or is capable of inhibiting cancer cell proliferation, or is capable of being endocytosed upon binding to ROR1.

119. A recombinant fusion protein comprising a ROR1-specific antigen binding of claim 115.

120. A recombinant fusion protein comprising an antigen binding molecule comprising an amino acid sequence represented by the formula (I):
FW1-CDR1-FW2-HV2-FW3a-HV4-FW3b-CDR3-FW4 (I)
wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region
or a functional variant thereof,
wherein the antigen binding molecule is fused to a fragment of an immunoglobulin Fc region wherein the fragment of an immunoglobulin Fc region is engineered to dimerize with a second fragment of an immunoglobulin Fc region,
optionally wherein:
(a) the fragment of an immunoglobulin Fc region is selected from the group consisting of an Fc heavy chain, a CH₂ region and a CH₃ region, further optionally wherein the fragment of an immunoglobulin Fc region is an Fc heavy chain;
(b) the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH₃ charge pairing, Fab-arm exchange, SEED technology, BEATtechnology, HA-TF, ZW1 approach, Biclonic approach, EW-RVT and Triomab; and/or
(c) one or more residues of the fragment of the immunoglobulin Fc region comprises one or more amino acid substitution suitable for knobs-in-holes (KIH) dimerization with a second fragment of an immunoglobulin Fc region comprising one or more corresponding amino acid mutation, further optionally wherein:
(i) the one or more amino acid substitution is selected from the group consisting of T366Y, Y407T, S354C, T366W, Y349C, T366S, L368A and Y407V; and/or,
(ii) the one or more amino acid substitution is selected from the group consisting of T366Y and Y407T.

121. The recombinant fusion protein of claim 120 comprising a sequence according to SEQ ID NO: 146, SEQ ID NO: 147, SEQ ID NO: 148, SEQ ID NO: 186, SEQ ID 187, SEQ ID NO: 194, SEQ ID NO: 195, SEQ ID NO: 196, SEQ ID NO: 191, SEQ ID NO: 192, SEQ ID NO: 197, SEQ ID NO: 198, SEQ ID NO: 199, SEQ ID NO: 165, SEQ ID NO: 166, SEQ ID NO: 200, SEQ ID NO: 201, SEQ ID NO: 202, SEQ ID NO: 167, SEQ ID NO: 188, SEQ ID NO: 189, SEQ ID NO: 203, SEQ ID NO: 204, or SEQ ID NO: 205.

122. A recombinant fusion protein dimer comprising
(a) a first recombinant fusion protein, wherein the first recombinant fusion protein is a recombinant fusion protein of claim 120, and
(b) a second recombinant fusion protein, wherein the second recombinant fusion protein comprises a second antigen binding molecule fused to a second fragment of an immunoglobulin Fc region engineered to dimerize with the first fragment of an immunoglobulin Fc region.

123. The recombinant fusion protein of claim 120, in which the ROR1-specific antigen binding molecule is fused to one or more biologically active proteins, optionally wherein:
(a) the specific antigen binding molecule is fused to one or more biologically active proteins via one or more linker domains, optionally, wherein the at least one biologically active protein is an immunoglobulin, an immunoglobulin Fc region, a fragment of an immunoglobulin Fc region, an Fc heavy chain, a CH₂ region, a CH₃ region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein;
(b) the Fc heavy chain is engineered to comprise one or more cysteine residues suitable for bioconjugation;
wherein (c) the fragment of an immunoglobulin Fc region is engineered to dimerize with a second fragment of an immunoglobulin Fc region;
(d) one or more residues of the fragment of the immunoglobulin Fc region comprises one or more amino acid substitution suitable for heterodimerization with a second fragment of an immunoglobulin Fc region comprising one or more corresponding amino acid mutation, optionally wherein the one or more amino acid substitution is selected from the group consisting of T366Y, Y407T, S354C, T366W, Y349C, T366S, L368A and Y407V,
(e) the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH₃ charge pairing, Fab-arm exchange, SEED technology, BEATtechnology, HA-TF, ZW1 approach, Biclonic approach, EW-RVT and Triomab.

124. A ROR1-specific chimeric antigen receptor (CAR), comprising at least one ROR1-specific antigen binding molecule as defined in claim 115, fused or conjugated to at least one transmembrane region and at least one intracellular domain.

125. A cell comprising a chimeric antigen receptor according to claim 124, which cell is preferably an engineered T-cell.

126. A pharmaceutical composition comprising the specific antigen binding molecule according to claim 115.

127. The specific antigen binding molecule according to claim 115 for use in the treatment of cancer, optionally wherein the cancer is a ROR1-positive cancer type, optionally wherein the cancer is selected from the group consisting of blood cancers such as lymphomas and leukaemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, oesophageal cancer, stomach cancer or liver cancer.

128. The specific antigen binding molecule according to claim 115, wherein the binding molecule comprises an amino acid sequence represented by the formula (II):
X-FW1-CDR1-FW2-HV2-FW3a-HV4-FW3b-CDR3-FW4-Y (II)
wherein
FW1-CDR1-FW2-HV2-FW3a-HV4-FW3b-CDR3-FW4 is a ROR1-specific antigen binding molecule;
X and Y are optional amino acid sequences
wherein the specific antigen binding molecule is conjugated to a second moiety. optionally, wherein:
(a) X or Y are individually either absent or selected from the group comprising an immunoglobulin, an immunoglobulin Fc region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein;
(b) the conjugation is via a cysteine residue in the amino acid sequence of the specific antigen binding molecule; and/or
(c) the second moiety is selected from
(i) the group comprising an immunoglobulin or antibody, an immunoglobulin Fc region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein; or
(ii) the group comprising detectable label, dye, toxin, drug, pro-drug, radionuclide or biologically active molecule, optionally wherein toxin is selected from the group comprising:
maytansinoids,
auristatins,
anthracyclines, preferably PNU-derived anthracyclines
amanitin derivatives, preferably α-amanitin derivatives
calicheamicins,
tubulysins
duocarmycins
radioisotopes—such as an alpha-emitting radionuclide, such as 227 Th or 225 Ac label
liposomes comprising a toxic payload,
protein toxins
taxanes
pyrrolbenzodiazepines and/or
indolinobenzodiazepine pseudodimers and/or
spliceosome inhibitors
CDK11 inhibitors
Pyridinobenzodiazepines
Irinotecan and its derivatives

129. A target-binding molecule-drug conjugate, comprising
(a) a ROR1 specific antigen binding molecule according to claim 115, and
(b) an anthracycline (PNU) derivative,
wherein the target-binding molecule-drug conjugate has the structure of formula (III): 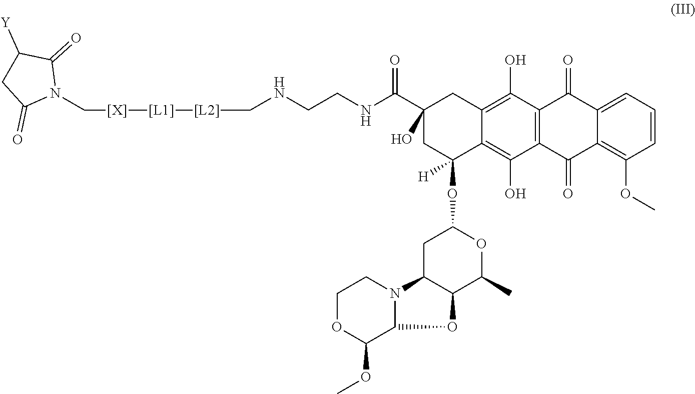
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises a ROR1 specific antigen binding molecule,
optionally wherein:
(a) the target-binding molecule-drug conjugate of formula (III) comprises [L1], [L2] or [L1] and [L2], further optionally wherein [L2] is p-aminobenzyloxycarbonyl (PAB) or alanine; or
(b) the target-binding molecule-drug conjugate has a structure selected from:

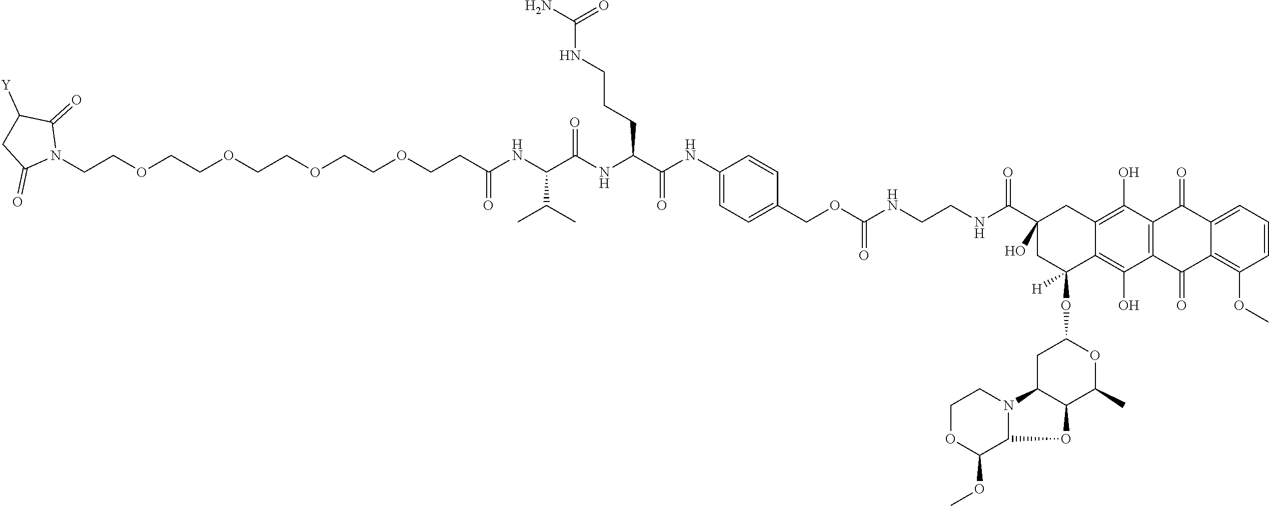

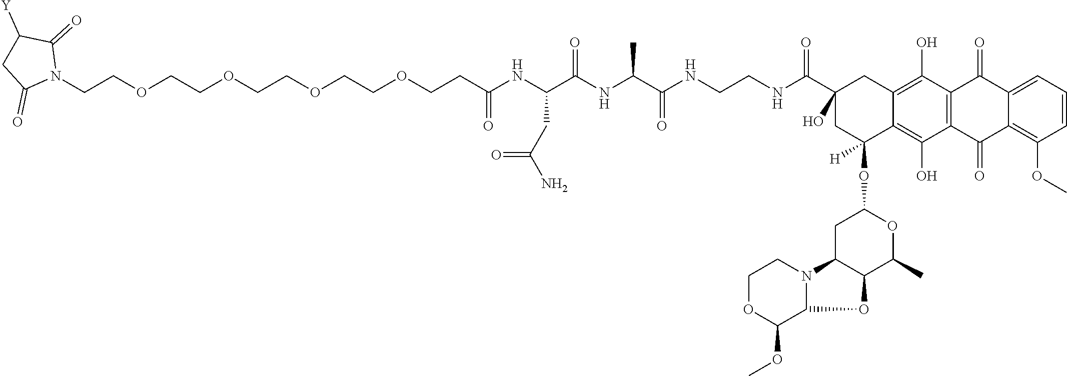

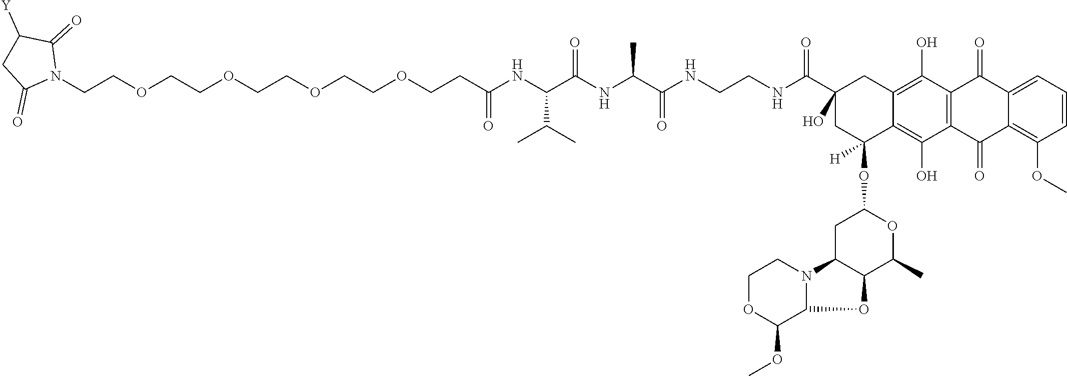

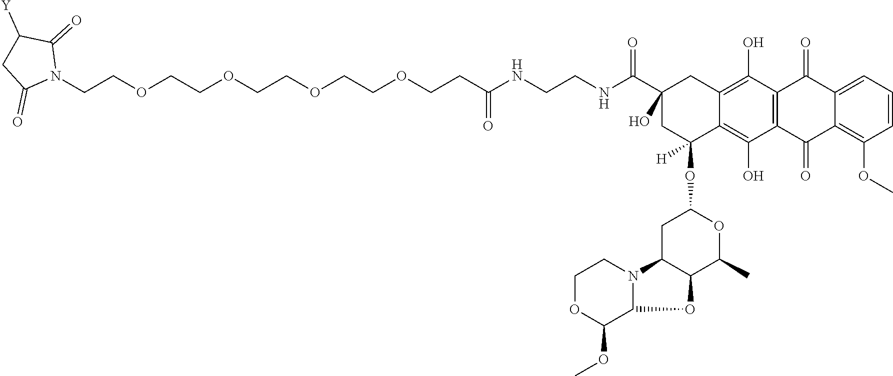

130. A target-binding molecule-drug conjugate, comprising
(a) a ROR1 specific antigen binding molecule according to claim 115, and
(b) an anthracycline (PNU) derivative,
wherein the target-binding molecule-drug conjugate has the structure of formula (IV): 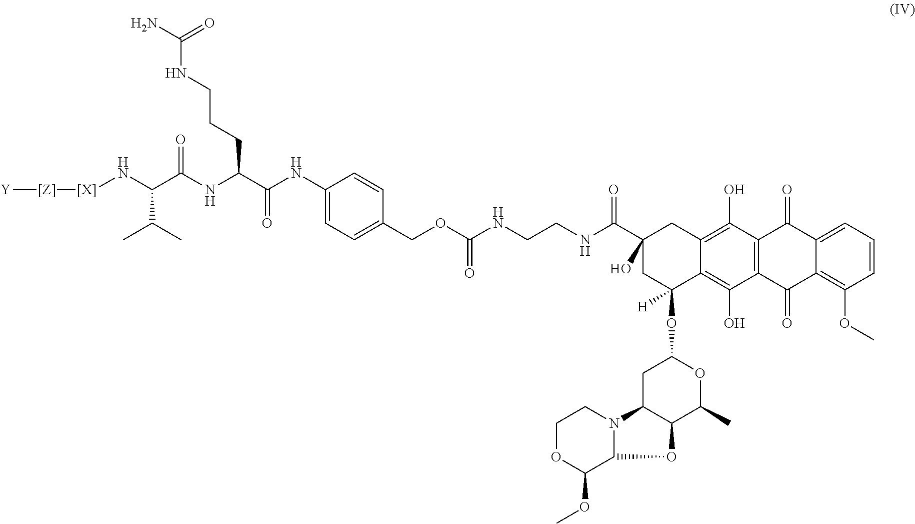
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y comprises the ROR1 specific antigen binding molecule
optionally wherein [Z] is selected from the group consisting of a disulphide bond, an amide bond, an oxime bond, a hydrazone bond, a thioether bond, a 1, 2, 3 triazole and polyGly.

131. The target-binding molecule-drug conjugate of claim 130, wherein:
(a) [X] is selected from the group comprising polyethylene glycol, 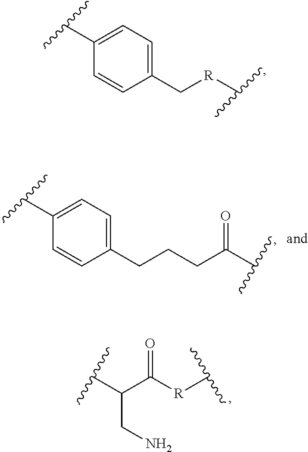
wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof; and/or
(b) wherein [X] is polyethylene glycol
(c) the target-binding molecule is a protein and the anthracycline (PNU) derivative is conjugated to a thiol-containing amino acid residue in the amino acid sequence of the protein, and/or wherein the PNU derivative is conjugated via a thiol moiety incorporated by chemical modification at the N-terminus or C-terminus of the amino acid sequence of the protein, and/or
(d) Y comprises a ROR1 specific antigen binding molecule conjugated to the PNU derivative via a human immunoglobulin Fc region or fragment thereof.

132. The specific antigen binding molecule of claim 131, wherein:
(a) the specific antigen binding molecule comprises SEQ ID NO: 178 and/or SEQ ID NO: 179;
(b) X or Y are individually either absent or selected from the group comprising an immunoglobin, an immunoglobulin Fc region, a fragment of an immunoglobulin Fc region, an Fc heavy chain, a CH₂ region, a CH₃ region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein;
(c) the immunoglobulin Fc region is an Fc heavy chain engineered to comprise one or more cysteine residues suitable for bioconjugation;
(d) X or Y are individually either absent or a fragment of an immunoglobulin Fc region engineered to dimerize with a second fragment of an immunoglobulin Fc region, optionally wherein the method is selected from the group consisting of knobs-into-holes (Y-T), knobs-into-holes (CW-CSAV), CH₃ charge pairing, Fab-arm exchange, SEED technology, BEATtechnology, HA-TF, ZW1 approach, Biclonic approach, EW-RVT and Triomab;
(e) X or Y are individually either absent or a fragment of the immunoglobulin Fc region that comprises one or more amino acid substitution suitable for heterodimerization with a second fragment of an immunoglobulin Fc region comprising one or more corresponding amino acid mutation, optionally wherein the one or more amino acid substitution is selected from the group consisting of T366Y, Y407T, S354C, T366W, Y349C, T366S, L368A and Y407V.
